(19) United States
(12) Patent Application Publication
(10) Pub. No.: US 2025/0302614 A1
(43) Pub. Date: Oct. 2, 2025

(54) PROSTHETIC VALVES AND SENSOR SYSTEMS

(71) Applicant: EDWARDS LIFESCIENCES CORPORATION, Irvine, CA (US)

(72) Inventors: Zachary Charles VanEvery, Lake Forest, CA (US); Rani Abdullah Mahmoudi, Huntington Beach, CA (US); Matthew Michael Becerra, Lake Forest, CA (US); Wei Gan, Irvine, CA (US); Kevin M. Stewart, Powell, TN (US); David Robert Landon, Huntington Beach, CA (US); Natan Anatoly Bukhdruker, Tirat Carmel (IL); Gil Hamerman, Kfar Yona (IL); Carmel Peleg, Pardes Hana-Karkur (IL)

(21) Appl. No.: 19/235,923

(22) Filed: Jun. 12, 2025

Related U.S. Application Data
(63) Continuation of application No. PCT/US2023/082880, filed on Dec. 7, 2023.
(60) Provisional application No. 63/432,830, filed on Dec. 15, 2022.

(51) Int. Cl. A61F 2/24; A61N 1/05

(52) U.S. Cl. CPC A61F 2/2418; A61F 2220/0016; A61F 2250/0002; A61F 2250/0096; A61N 1/0573

(57) ABSTRACT

Apparatuses, systems, and methods for prosthetic valves having sensor systems are disclosed. Examples of prosthetic valves include replacement heart valves for replacing the function of a native heart valve, such as a mitral or tricuspid valve. In various embodiments, replacement heart valves are provided with sensors or markers for assisting with proper placement and/or securement in the body. A replacement heart valve may include anchors for securing the replacement heart valve to native leaflets, wherein sensors and/or markers provide feedback to the physician for confirming proper placement of the anchors during the implantation procedure. Sensors may also be used to assess the function of the prosthetic valve after deployment. Sensors may also be used to monitor blood pressure, temperature, oxygen, insulin, cholesterol, and/or glucose to assess overall patient health.

### CROSS REFERENCE TO RELATED APPLICATIONS

This application is a continuation of International Application No. PCT/US2023/082880, filed Dec. 7, 2023, which designates the United States and was published in English by the International Bureau on Jun. 20, 2024, which claims the benefit of U.S. Provisional Application No. 63/432,830, filed Dec. 15, 2022, the entire contents of each of which are hereby incorporated by reference.

### BACKGROUND

### Field

Certain features of the disclosure relate to implants, including prosthetic valves for deployment. Certain features of the disclosure relate to sensor systems for facilitating implantation and/or assessing the function of implants.

### Background

Human heart valves, which include the aortic, pulmonary, mitral, and tricuspid valves, function as one-way valves operating in synchronization with the pumping heart. The valves allow blood to flow downstream, but block blood from flowing upstream. Diseased heart valves exhibit impairments such as narrowing of the valve or regurgitation, which inhibit the valves' ability to control blood flow. Such impairments reduce the heart's blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures.

These replacement valves are desirably deployed to an implantation site in a desired configuration. For example, considerations such as anchoring the replacement valve, sealing of the replacement valve with the native valve, and proper operation of the prosthetic valve leaflets may be at issue upon deployment to the implantation site. Further, sensing of one or more conditions within a patient's body may be desirable at a time of implantation of a replacement valve or following such implantation.

### SUMMARY

Examples of prosthetic valves and sensor systems disclosed herein are directed to improvements in prosthetic valves and sensor systems. Examples of prosthetic valves include replacement heart valves. Features disclosed herein may be utilized for improved identification of proper anchoring, positioning, and other conditions during the implantation procedure. Features disclosed herein may also assist with the assessment of proper implant function after implantation. Implantable sensor systems disclosed herein may also be used to monitor blood conditions, such as pressure, temperature, oxygen, insulin, platelets, cholesterol, and/or glucose, for assessing patient health. Various other improvements are also disclosed.

In aspects, a prosthetic valve is provided for implantation within a native valve. The prosthetic valve may include a valve body, one or more prosthetic valve leaflets coupled to the valve body, and one or more anchor mechanisms adapted for securing the valve body to surrounding tissue. In preferred embodiments, the anchor mechanisms are shaped to capture one or more native valve leaflets between the anchoring mechanisms and the valve body. The prosthetic valve preferably also includes one or more indicators (e.g., sensors) for providing feedback to the physician regarding capture of the native valve leaflet and/or proper placement of the one or more anchors with respect to the native valve. The feedback is preferably provided to the physician via visualization, preferably in real-time, using medical imaging techniques, such as ultrasound or fluoroscopy.

In aspects, a method may include deploying a prosthetic valve to a native valve. The prosthetic valve may include a valve body, one or more prosthetic valve leaflets coupled to the valve body, one or more anchors adapted to anchor the valve body to the native valve by capturing a native valve leaflet, and an indicator adapted to indicate capture of the native valve leaflet.

In aspects, a sensor system may include a prosthetic heart valve for deployment to a native valve of a patient's heart. One or more sensors are coupled to the prosthetic heart valve for detecting a condition within the patient's body.

In aspects, a method may include deploying a sensor system to a native valve. The sensor system may include a prosthetic heart valve for deployment to a native valve of a patient's heart, and one or more sensors adapted to be coupled to the prosthetic heart valve and adapted to detect a condition within the patient's body.

In aspects, a delivery system is provided for delivering an implant to a native heart valve. The delivery system may include a delivery apparatus for delivering the implant to the native heart valve; and one or more sensors coupled to the delivery apparatus and adapted to sense a spatial relationship between the delivery apparatus and at least a portion of the native heart valve.

In aspects, a method may include delivering an implant to a native heart valve utilizing a delivery system. The delivery system may include a delivery apparatus for delivering the implant to the native heart valve, and one or more sensors coupled to the delivery apparatus and adapted to sense a spatial relationship between the delivery apparatus and at least a portion of the native heart valve.

In aspects, a delivery system is provided for delivering an implant to a native heart valve. The delivery system may include a delivery apparatus for delivering the implant to the native heart valve. An imaging apparatus may be coupled to the delivery for imaging an area (e.g., surrounding tissue) external to the delivery apparatus.

In aspects, a method may include delivering an implant to a native heart valve utilizing a delivery system. The delivery system may include a delivery apparatus for delivering the implant to the native heart valve, and an imaging apparatus coupled to the delivery apparatus and adapted to image an area external to the delivery apparatus.

In aspects, a sensor system may include a sensor and one or more anchors coupled to the sensor and adapted to engage an interior heart wall of a chamber of a heart.

In aspects, a method may include deploying a sensor system to a native valve. The sensor system may include a sensor, and one or more anchors coupled to the sensor and adapted to engage an interior heart wall of a chamber of a heart to anchor the sensor to the interior heart wall.

In aspects, a system may include a prosthetic heart valve for deployment to a native valve of a patient's heart, at least a portion of the prosthetic heart valve comprising a pacemaker electrical conduit adapted to conduct an electrical signal for pacing a heart.

In aspects, a method may include deploying a prosthetic heart valve to a native valve of a patient's heart, at least a portion of the prosthetic heart valve comprising a pacemaker electrical conduit adapted to conduct an electrical signal for pacing a heart.

In aspects, a system may include a prosthetic heart valve for deployment to a native valve of a patient's heart, the prosthetic heart valve including one or more anchors adapted to hook around one or more native valve leaflets to anchor the prosthetic heart valve to the native valve. The system may include a delivery catheter for delivering the prosthetic heart valve to the native valve. The system may include a retainer mechanism adapted to retain the one or more native valve leaflets in a contracted state upon the one or more anchors at least partially hooking around the one or more native valve leaflets.

In aspects, a method may include deploying a prosthetic heart valve to a native valve of a patient's heart utilizing a delivery catheter, the prosthetic heart valve including one or more anchors adapted to hook around one or more native valve leaflets to anchor the prosthetic heart valve to the native valve. The method may include utilizing a retainer mechanism to retain the one or more native valve leaflets in a contracted state when the one or more anchors at least partially hook around the one or more native valve leaflets.

In aspects, a prosthetic heart valve is provided for deployment to a native valve. The prosthetic valve may include one or more prosthetic valve leaflets (e.g., made from pericardium) for providing one-way valve function. The prosthetic valve may include an inner frame supporting the one or more prosthetic valve leaflets and having an inflow end portion and an outflow end portion. The prosthetic valve may include a sealing body positioned radially outward of the inner frame and including a plurality of elongate prongs and a skirt, the plurality of elongate prongs each having a first end portion coupled to the inflow end portion of the inner frame and protruding radially outward from the inner frame to a second end portion, the skirt being suspended between the second end portions of the plurality of prongs and an outflow end portion of the prosthetic valve, the skirt bounding a pocket positioned between the skirt and the inner frame. The prosthetic valve may include one or more anchors adapted to anchor the prosthetic valve to the native valve by capturing a native valve leaflet.

In aspects, a method may include deploying a prosthetic heart valve to a native heart valve. The prosthetic heart valve may include one or more prosthetic valve leaflets, an inner frame supporting the one or more prosthetic valve leaflets and having an inflow end portion and an outflow end portion, a sealing body positioned radially outward of the inner frame and including a plurality of elongate prongs and a skirt, the plurality of elongate prongs each having a first end portion coupled to the inflow end portion of the inner frame and protruding radially outward from the inner frame to a second end portion, the skirt being suspended between the second end portions of the plurality of prongs and an outflow end portion of the prosthetic valve, the skirt bounding a pocket positioned between the skirt and the inner frame, and one or more anchors adapted to anchor the prosthetic heart valve to the native heart valve by capturing a native valve leaflet.

In aspects, a prosthetic heart valve is provided for deployment to a native valve. The prosthetic valve may include one or more prosthetic valve leaflets. The prosthetic valve may include a support structure for supporting the one or more prosthetic valve leaflets and including at least one ring coupled to a skirt, the skirt or the at least one ring being adapted to seal with at least a portion of the native valve.

In aspects, a method may include deploying a prosthetic heart valve to a native heart valve. The prosthetic heart valve may include one or more prosthetic valve leaflets. The prosthetic heart valve may include a support structure for supporting the one or more prosthetic valve leaflets and including at least one ring coupled to a skirt, the skirt or the at least one ring being adapted to seal with at least a portion of the native valve.

In aspects, a sensor system may be incorporated into a prosthetic heart implant. The sensor system may include a sensor body including: a substrate, a sensor positioned on the substrate and adapted to detect a condition of the prosthetic heart implant, and an electrical detection trace positioned on the substrate and adapted to detect a force applied to the substrate.

In aspects, a method may include deploying a prosthetic heart implant to a native heart valve; and utilizing a sensor body coupled to the prosthetic heart implant to detect a condition of the prosthetic heart implant, the sensor body including: a substrate, a sensor positioned on the substrate and adapted to detect the condition of the prosthetic heart implant, and an electrical detection trace positioned on the substrate and adapted to detect a force applied to the substrate.

Any of the features of an aspect or example disclosed herein, is applicable to all other aspects and examples identified herein. Moreover, any of the features of an aspect or example of the various aspects or examples, is independently combinable, partly or wholly with other aspects or examples described herein in any way, e.g., one, two, or three or more aspects or examples may be combinable in whole or in part. Further, any of the features of an aspect or example may be made optional to other aspects or examples. Any aspect or example of a method can be performed by a system or apparatus of another aspect or example, and any aspect or example of a system or apparatus can be configured to perform a method of another aspect or example.

### BRIEF DESCRIPTION OF THE DRAWINGS

Features and advantages of the systems, apparatuses, and methods as disclosed herein will become appreciated as the same become better understood with reference to the specification, claims, and appended drawings wherein:

FIG. 1A illustrates an upper perspective view of a prosthetic valve according to examples of the present disclosure.

FIG. 1B illustrates a bottom perspective view of the prosthetic valve shown in FIG. 1A.

FIG. 2 illustrates a side cross-sectional schematic view of the prosthetic valve shown in FIG. 1A.

FIG. 3 illustrates a schematic view of a delivery apparatus approaching an implantation site.

FIG. 4A illustrates a side cross-sectional schematic view of the prosthetic valve shown in FIG. 1A positioned within a delivery apparatus and approaching an implantation site.

FIG. 4B illustrates a side cross-sectional schematic view of the prosthetic valve shown in FIG. 1A deployed to a native heart valve.

FIG. 4C illustrates a side cross-sectional schematic view of the prosthetic valve shown in FIG. 1A deployed to a native heart valve.

FIG. 5 illustrates an upper perspective view of a prosthetic valve according to examples of the present disclosure.

FIG. 6 illustrates a side cross-sectional schematic view of the prosthetic valve shown in FIG. 5 deployed to a native heart valve.

FIG. 7 illustrates an upper perspective view of a prosthetic valve according to examples of the present disclosure.

FIG. 8A illustrates a side cross-sectional schematic view of the prosthetic valve shown in FIG. 7 positioned within a delivery apparatus and approaching an implantation site.

FIG. 8B illustrates a side cross-sectional schematic view of the prosthetic valve shown in FIG. 7 partially deployed from a delivery apparatus and approaching an implantation site.

FIG. 8C illustrates a side cross-sectional schematic view of the prosthetic valve shown in FIG. 7 partially deployed from a delivery apparatus and approaching an implantation site.

FIG. 8D illustrates a side cross-sectional schematic view of the prosthetic valve shown in FIG. 7 partially deployed from a delivery apparatus and approaching an implantation site.

FIG. 8E illustrates a side cross-sectional schematic view of the prosthetic valve shown in FIG. 7 partially deployed from a delivery apparatus.

FIG. 8F illustrates a side cross-sectional schematic view of the prosthetic valve shown in FIG. 7 deployed to a native heart valve.

FIG. 9 illustrates an upper perspective view of a prosthetic valve according to examples of the present disclosure.

FIG. 10 illustrates a side cross-sectional schematic view of the prosthetic valve shown in FIG. 9.

FIG. 11 illustrates an upper perspective view of a prosthetic valve according to examples of the present disclosure.

FIG. 12 illustrates a top cross-sectional schematic view of the prosthetic valve shown in FIG. 11, deployed to native heart valve leaflets.

FIG. 13 illustrates an upper perspective view of a prosthetic valve according to examples of the present disclosure.

FIG. 14 illustrates a side cross-sectional schematic view of the prosthetic valve shown in FIG. 13.

FIG. 15 illustrates an upper perspective view of a prosthetic valve according to examples of the present disclosure.

FIG. 16 illustrates a side cross-sectional schematic view of the prosthetic valve shown in FIG. 15.

FIG. 17 illustrates an upper perspective view of a prosthetic valve according to examples of the present disclosure.

FIG. 18 illustrates a side cross-sectional schematic view of the prosthetic valve shown in FIG. 17.

FIG. 19 illustrates an upper perspective view of a prosthetic valve according to examples of the present disclosure.

FIG. 20 illustrates a side cross-sectional schematic view of the prosthetic valve shown in FIG. 19.

FIG. 21 illustrates an upper perspective view of a prosthetic valve according to examples of the present disclosure.

FIG. 22 illustrates a side perspective view of an indicator of the prosthetic valve shown in FIG. 21 according to examples of the present disclosure.

FIG. 23A illustrates a side cross-sectional schematic view of the indicator shown in FIG. 22.

FIG. 23B illustrates a side cross-sectional schematic view of the indicator shown in FIG. 23A deflected from the position shown in FIG. 23A.

FIG. 24 illustrates a side cross-sectional schematic view of a prosthetic valve according to examples of the present disclosure.

FIG. 25 illustrates a side cross-sectional schematic view of a prosthetic valve according to examples of the present disclosure.

FIG. 26 illustrates an upper perspective view of a prosthetic valve according to examples of the present disclosure.

FIG. 27 illustrates a side cross-sectional schematic view of the prosthetic valve shown in FIG. 26 being deployed to an implantation site according to examples of the present disclosure.

FIG. 28 illustrates a perspective view of a delivery apparatus according to examples of the present disclosure.

FIG. 29 illustrates a side cross-sectional schematic view of the prosthetic valve shown in FIG. 26 deployed to an implantation site according to examples of the present disclosure.

FIG. 30 illustrates an upper perspective view of a prosthetic valve according to examples of the present disclosure.

FIG. 31 illustrates a side cross-sectional schematic view of the prosthetic valve shown in FIG. 30 deployed to an implantation site according to examples of the present disclosure.

FIG. 32A illustrates a side cross-sectional schematic view of the prosthetic valve shown in FIG. 30 deployed to an implantation site according to examples of the present disclosure.

FIG. 32B illustrates a side cross-sectional schematic view of a prosthetic valve according to examples of the present disclosure.

FIG. 33 illustrates an upper perspective view of a prosthetic valve according to examples of the present disclosure.

FIG. 34 illustrates a side cross-sectional schematic view of the prosthetic valve shown in FIG. 33 deployed to an implantation site according to examples of the present disclosure.

FIG. 35 illustrates a side cross-sectional schematic view of a prosthetic valve according to examples of the present disclosure.

FIG. 36 illustrates a side perspective view of a sensor system according to examples of the present disclosure.

FIG. 37 illustrates a side perspective view of a sensor system according to examples of the present disclosure.

FIG. 38 illustrates a side cross-sectional schematic view of a prosthetic valve according to examples of the present disclosure.

FIG. 39 illustrates an upper perspective view of a prosthetic valve according to examples of the present disclosure.

FIG. 40 illustrates a side cross-sectional schematic view of a prosthetic valve according to examples of the present disclosure.

FIG. 41 illustrates an upper perspective view of a plurality of sensors according to examples of the present disclosure.

FIG. 42A illustrates a top view of a sensor according to examples of the present disclosure.

FIG. 42B illustrates a side cross-sectional schematic view of the sensor shown in FIG. 42A according to examples of the present disclosure.

FIG. 42C illustrates a side cross-sectional schematic view of the sensor shown in FIG. 42A according to examples of the present disclosure.

FIG. 43 illustrates a side cross-sectional schematic view of a prosthetic valve utilizing the sensors shown in FIG. 41.

FIG. 44 illustrates a top view of a sensor for a prosthetic valve according to examples of the present disclosure.

FIG. 45 illustrates a side cross-sectional schematic view of a prosthetic valve according to examples of the present disclosure.

FIG. 46 illustrates a side view of a delivery apparatus according to examples of the present disclosure.

FIG. 47 illustrates a side view of the delivery apparatus shown in FIG. 46 moved distally.

FIG. 48 illustrates a side view of the delivery apparatus shown in FIG. 46 with an implant partially deployed.

FIG. 49 illustrates a side view of a delivery apparatus according to examples of the present disclosure.

FIG. 50A illustrates a side view of an imaging apparatus extending from a delivery system.

FIG. 50B illustrates a side view of an imaging apparatus extending from a delivery system.

FIG. 51 illustrates a side perspective view of an implant having imaging windows.

FIG. 52 illustrates a side perspective view of a sensor for coupling to an interior heart wall.

FIG. 53 illustrates a schematic view of a ventricle and atrium.

FIG. 54 illustrates a side view of a sensor being anchored to an interior heart wall.

FIG. 55 illustrates a side view of a sensor being anchored to an interior heart wall.

FIG. 56 illustrates an upper perspective view of a prosthetic valve according to examples of the present disclosure.

FIG. 57 illustrates a side cross-sectional schematic view of the prosthetic valve shown in FIG. 56.

FIG. 58 illustrates a side cross-sectional schematic view of the prosthetic valve shown in FIG. 56 deployed to a prosthetic heart valve.

FIG. 59 illustrates a side cross-sectional schematic view of a pacemaker coupled to an electrical terminal of a prosthetic heart valve.

FIG. 60 illustrates a side perspective view of a pacemaker electrical conduit according to examples of the present disclosure.

FIG. 61 illustrates a side perspective view of a pacemaker electrical conduit according to examples of the present disclosure.

FIG. 62A illustrates a side partial cross-sectional view of an approach of a retainer mechanism to an implantation site.

FIG. 62B illustrates a side partial cross-sectional view of the retainer mechanism shown in FIG. 62A with the retainer mechanism deployed.

FIG. 62C illustrates a side partial cross-sectional view of the retainer mechanism shown in FIG. 62A with the retainer mechanism deployed.

FIG. 62D illustrates a side partial cross-sectional view of the retainer mechanism shown in FIG. 62A with the retainer mechanism retracted.

FIG. 62E illustrates a perspective view of the retainer mechanism shown in FIG. 62A with the retainer mechanism deployed.

FIG. 62F illustrates a top cross-sectional view of the retainer mechanism shown in FIG. 62A with the retainer mechanism deployed.

FIG. 63A illustrates a side view of an approach of a retainer mechanism to an implantation site.

FIG. 63B illustrates a side partial cross-sectional view of the retainer mechanism shown in FIG. 63A deployed.

FIG. 63C illustrates a side partial cross-sectional view of the retainer mechanism shown in FIG. 63A retracted.

FIG. 64 illustrates a side partial cross-sectional view of a retainer mechanism deployed.

FIG. 65A illustrates a side partial cross-sectional view of a retainer mechanism deployed.

FIG. 65B illustrates a side partial cross-sectional view of the retainer mechanism shown in FIG. 65A deployed.

FIG. 65C illustrates a side partial cross-sectional view of the retainer mechanism shown in FIG. 65A retracted.

FIG. 66A illustrates a side partial cross-sectional view of a retainer mechanism retracted.

FIG. 66B illustrates a side partial cross-sectional view of the retainer mechanism shown in FIG. 66A deployed.

FIG. 66C illustrates a side partial cross-sectional view of the retainer mechanism shown in FIG. 66A retracted.

FIG. 66D illustrates a perspective view of the retainer mechanism shown in FIG. 66A.

FIG. 67 illustrates a side partial cross-sectional view of a retainer mechanism.

FIG. 68A illustrates a side partial cross-sectional view of a retainer mechanism.

FIG. 68B illustrates a side partial cross-sectional view of the retainer mechanism shown in FIG. 68A with a coil deployed.

FIG. 68C illustrates a perspective view of the coil shown in FIG. 68B deployed.

FIG. 69A illustrates a perspective view of a frame of a prosthetic valve.

FIG. 69B illustrates a top view of the frame shown in FIG. 69A.

FIG. 69C illustrates a perspective view of a prosthetic valve utilizing the frame shown in FIG. 69A.

FIG. 69D illustrates a perspective view of an inner frame.

FIG. 69E illustrates a plan view of an elongate strut.

FIG. 69F illustrates a side cross-sectional view of the prosthetic valve shown in FIG. 69C.

FIG. 69G illustrates a side cross-sectional view of the prosthetic valve shown in FIG. 69C deployed.

FIG. 70A illustrates a perspective view of a prosthetic valve.

FIG. 70B illustrates a side cross-sectional view of a portion of the prosthetic valve shown along line I-I in FIG. 70A.

FIG. 70C illustrates a top view of a ring.

FIG. 70D illustrates a side cross-sectional view of the prosthetic valve shown in FIG. 70A deployed.

FIG. 70E illustrates a top view of a ring.

FIG. 70F illustrates a partial perspective view of a ring positioned within a channel.

FIG. 71A illustrates a perspective view of a prosthetic valve.

FIG. 71B illustrates a side cross-sectional view of the prosthetic valve shown in FIG. 71A.

FIG. 71C illustrates a side cross-sectional view of the prosthetic valve shown in FIG. 71A.

FIG. 72A illustrates a perspective view of a prosthetic valve.

FIG. 72B illustrates a side cross-sectional view of the prosthetic valve shown in FIG. 72A deployed.

FIG. 73A illustrates a perspective view of a prosthetic valve.

FIG. 73B illustrates a side cross-sectional view of the prosthetic valve shown in FIG. 73A deployed.

FIG. 74 illustrates a side view of a delivery system.

FIG. 75 illustrates a side view of a clip.

FIG. 76 illustrates a partial cross-sectional view of the clip shown in FIG. 75 being implanted.

FIG. 77 illustrates a partial cross-sectional view of the clip shown in FIG. 75 being implanted.

FIG. 78 illustrates a partial cross-sectional view of the clip shown in FIG. 75 implanted.

FIG. 79 illustrates a cross-sectional view of a clip being implanted.

FIG. 80 illustrates a cross-sectional view of a clip being implanted.

FIG. 81 illustrates a perspective view of a sensor body.

FIG. 82 illustrates a partial schematic view of components of a sensor system.

FIG. 83 illustrates a top view of a partial tear of the sensor body shown in FIG. 81.

FIG. 84 illustrates a top view of a sensor body.

FIG. 85 illustrates a top view of a sensor body.

FIG. 86 illustrates a schematic view of a delivery system approaching an implantation site.

FIG. 87 illustrates a perspective view of a prosthetic heart valve including a sensor body.

FIG. 88 illustrates a top view of a sensor body compressed.

FIG. 89 illustrates a top view of the sensor body shown in FIG. 88 expanded.

FIG. 90 illustrates a perspective view of the sensor body shown in FIG. 88 expanded.

FIG. 91 illustrates a perspective view of the sensor body shown in FIG. 88 compressed.

FIG. 92 illustrates a perspective view of a prosthetic heart valve including a sensor body.

### DETAILED DESCRIPTION

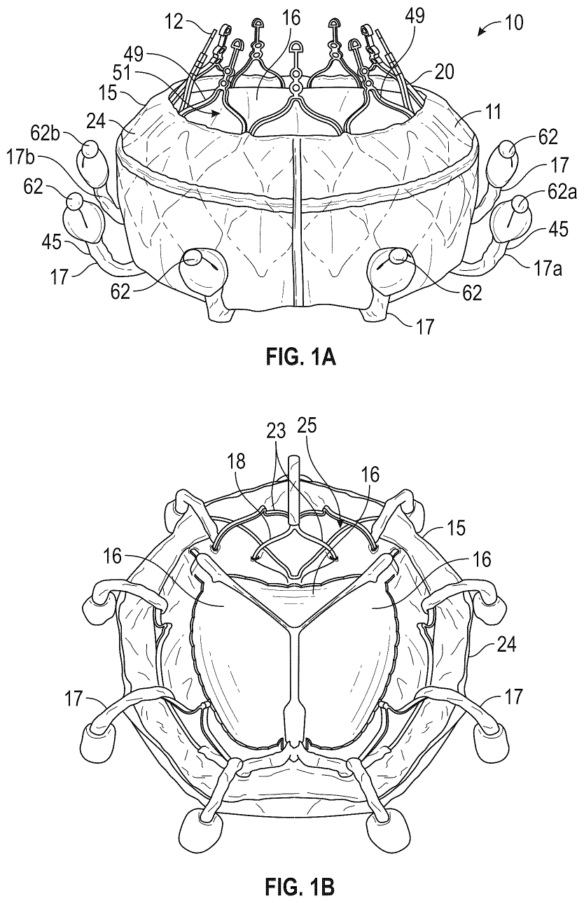

FIG. 1A illustrates a perspective view of an implant in the form of a prosthetic valve 10. The prosthetic valve 10 may comprise a prosthetic heart valve for deployment to a native heart valve of a patient's body. In examples, other forms of implants and prosthetic valves may be utilized as desired.

The prosthetic valve 10 is adapted to be deployed within an annulus of a native valve, such as a native mitral valve or a native tricuspid valve. In examples, other implantation locations can be utilized such as within an aortic or pulmonary valve, or in other valves or locations within a patient's body as desired.

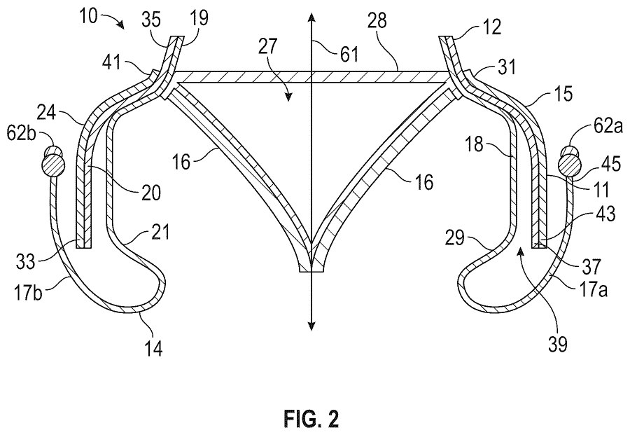

The prosthetic valve 10 includes a proximal end 12 or inlet end portion and a distal end 14 or outlet end portion (marked in FIG. 2), and a length therebetween. The prosthetic valve 10 further includes a valve portion, preferably formed by a plurality of prosthetic valve leaflets 16. The valve portion is positioned in a flow channel or passageway for controlling flow through the prosthetic valve 10. The flow channel or passageway is formed by a support structure or valve body 15 of the valve 10. The valve body 15 or support structure has a proximal end portion or inlet end portion and a distal end portion or outlet end portion. The prosthetic valve leaflets 16 move between opened and closed states to mimic and replace the operation of native valve leaflets. The valve portion is positioned within the passageway of the valve body 15 for permitting flow of blood through the passageway in one direction, thereby replacing the function of a native heart valve. The prosthetic valve leaflets 16 are made of pericardium, such as bovine or porcine pericardium, or another material as desired. In alternative arrangements, the leaflets are formed of a synthetic (e.g., polymer) material or the valve portion is a mechanical one-way valve.

The valve body 15 or support structure surrounds and supports the valve portion and the one or more prosthetic valve leaflets 16. The valve body 15 includes a stent or a frame or a support frame (e.g., a valve frame or inner support stent or inner frame 18 (as shown in FIG. 1B) and an outer support stent or outer frame 20 (as shown in FIGS. 1A and 2), among other forms of frames). The outer support stent or outer frame 20 is part of a sealing body 11 and is spaced from the inner frame 18. The outer frame 20 surrounds the inner frame 18.

FIG. 2 illustrates a cross-sectional schematic view of the prosthetic valve 10. The inner frame 18 includes a proximal portion including a proximal end 19 and a distal portion including a distal end 21. The inner frame 18 has a bulb shape, comprising a curved body that curves radially outward between the proximal end 19 and the distal end 21, or has another configuration in examples as desired. The inner frame 18 has a circular shape in examples. The inner frame 18 supports the plurality of prosthetic valve leaflets 16.

Referring to FIG. 1B, the inner frame 18 includes a plurality of struts 23 spaced from each other with spaces 25. Such a configuration allows the inner frame 18 to move between an undeployed, unexpanded, or linearized configuration to a deployed or expanded configuration. For example, the inner frame 18 expands radially outward to move to the deployed or expanded configuration, with the length of the inner frame 18 decreasing due to the increased diameter of the inner frame 18. Other configurations of inner frames 18 can be utilized as desired.

The valve body 15 includes a sealing body 11. The sealing body 11 is positioned radially outward from the prosthetic valve leaflets 16 and is adapted to seal against a portion of the native valve. The sealing body 11 comprises the outer surface of the prosthetic valve 10. The sealing body 11 defines the outer diameter of the prosthetic valve 10 and comprises the outer periphery of the prosthetic valve 10. The sealing body 11 includes a proximal portion having a proximal end 31 and includes a distal portion having a distal end 33 (marked in FIG. 2).

Referring to the cross-sectional view of FIG. 2, the sealing body 11 includes a frame 20 and a sealing skirt 24, or in examples comprises only a frame or only a sealing skirt as desired. The frame 20 comprises an outer frame that is positioned radially outward from the inner frame 18. The sealing skirt 24 is coupled to the outer frame 20 and comprises the outer portion of the sealing body 11 as shown in FIG. 1A.

The outer frame 20 comprises at least a portion of the sealing body 11 that is adapted to apply a seal to a portion of a heart. The outer frame 20 has a proximal portion 35 that couples to the proximal end 19 of the inner frame 18. The proximal portion 35 extends radially outward from the proximal end 19 of the inner frame 18 and from the prosthetic valve leaflets 16. A distal portion 37 of the outer frame 20 is spaced from the prosthetic valve leaflets 16 and the inner frame 18 at a gap 39. The gap 39 is positioned between the outer frame 20 of the sealing body 11 and a distal portion of the inner frame 18. The inner frame 18 accordingly comprises an inner frame and the frame 20 of the sealing body 11 comprises an outer frame positioned radially outward of the inner valve frame 18 and surrounding the inner frame 18 and the prosthetic valve leaflets 16.

The outer frame 20 has a length that extends distally to a lesser distance than the distal end of the inner frame 18. As such, the outer frame 20 is shorter than the inner frame 18. The outer frame 20 has a curved configuration that curves outward from the inner frame 18, with the greatest diameter of the outer frame 20 being at the distal portion of the outer frame 20.

The outer frame 20 of the sealing body 11 includes a plurality of struts 49 (as marked in FIG. 1A) forming the frame 20, with spaces 51 between the struts. Such a configuration utilized with the frame 20 allows the frame 20 to move between an undeployed, unexpanded, or linearized configuration to a deployed or expanded configuration as shown in FIG. 1A, in which the outer frame 20 and sealing body 11 have a curved bulbous shape. As with the valve frame 18, the length of the outer frame 20 of the sealing body 11 decreases as the diameter of the outer frame 20 of the sealing body 11 increases during deployment. The diameter of the outer frame 20 of the sealing body 11 can radially expand outward from the inner valve frame 18 simultaneously, or at a different time or rate of expansion as the inner valve frame 18 in examples.

The sealing body 11 includes a sealing skirt 24 (as shown in FIG. 1A) that extends around the inner valve frame 18 and the prosthetic valve leaflets 16. The skirt 24 can be coupled to the frame 20 of the sealing body or can be free from the frame 20 in examples.

The sealing skirt 24 has a proximal portion 41 (marked in FIG. 2) that is coupled to the proximal portion of the frame 20 of the sealing body 11 and can be coupled to the proximal portion of the inner frame 18. The skirt 24 has a distal portion 43 (marked in FIG. 2) that can be coupled to the distal end of the frame 20, and in examples can be coupled to the inner valve frame 18 or one or more anchors 17.

The sealing skirt 24 is made of a material that resists fluid flow therethrough, such as a cloth material, woven material, or other material such as a polymer or other material that resists fluid flow therethrough. The material may comprise a fabric. A variety of materials can be utilized for the skirt 24 as desired.

The sealing body 11 is adapted to abut a portion of the patient's heart to reduce fluid flow. The skirt 24 is adapted to seal a portion of the native valve annulus. For example, the sealing body 11 abuts a surface of a patient's native valve leaflet to reduce fluid flow between the sealing body 11 and the native leaflet. The sealing body 11 can be adapted to abut other portions of the patient's heart to reduce fluid flow as desired.

In examples, the sealing body 11 is flexible to allow for movement and conformability to a native valve annulus.

Referring to FIGS. 1A and 2, the prosthetic valve 10 includes one or more anchors 17. The anchors 17 can each be adapted to anchor the prosthetic valve leaflets 16 to a portion of a patient's heart, which comprises a native valve. The anchors 17 can particularly be adapted to anchor to the native valve leaflets of the patient's heart. The anchors 17 can extend around the native valve leaflets to anchor to (i.e., capture) the native valve leaflets. The anchors 17 comprise distal anchors positioned at the distal end 14 of the valve 10, or in examples can be positioned in another position as desired.

Each anchor 17 is configured as a protruding arm adapted to extend distally and then curve in a proximal direction to the tip of the respective one of the anchors 17. Such a configuration allows the anchor 17 to extend around a native leaflet and around the distal tip of the leaflet, to hook over the distal tip of the native valve leaflet and be positioned radially outward of an outward facing surface of a leaflet of the native valve. The anchors 17 are adapted to be in a hooked configuration as shown in FIGS. 1A-2 for example. If desired, the anchors 17 are adapted to clamp one or more native leaflets against the sealing body 11. When implanted within a native mitral or tricuspid valve, the anchor 17 can thus resist a force applied in the atrial or proximal direction to the valve 10 and anchors the valve 10 within the native valve annulus. Other configurations of anchors 17 can be utilized in examples as desired.

The anchors 17 anchor the valve body 15 to a native valve by capturing a native valve leaflet. Capturing has a variety of forms, including extending over a distal tip of a native valve leaflet. The anchors 17 hook around the leaflets in examples. Other forms of capture can be utilized in examples.

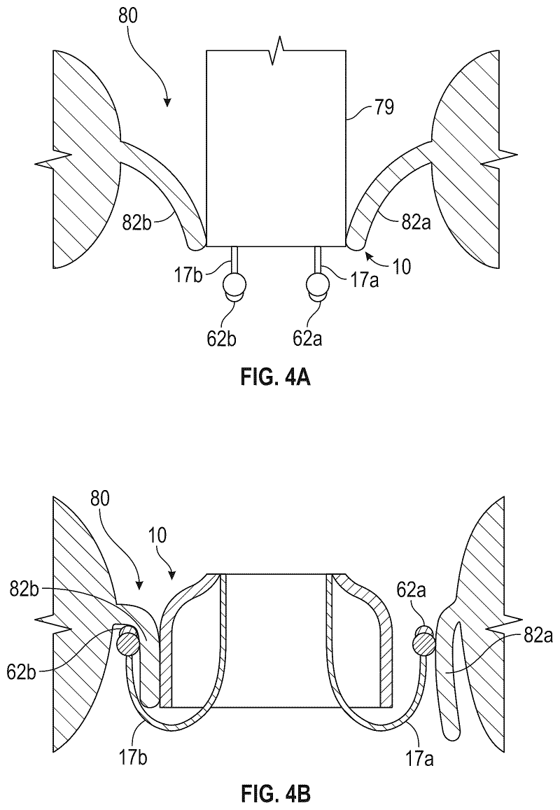

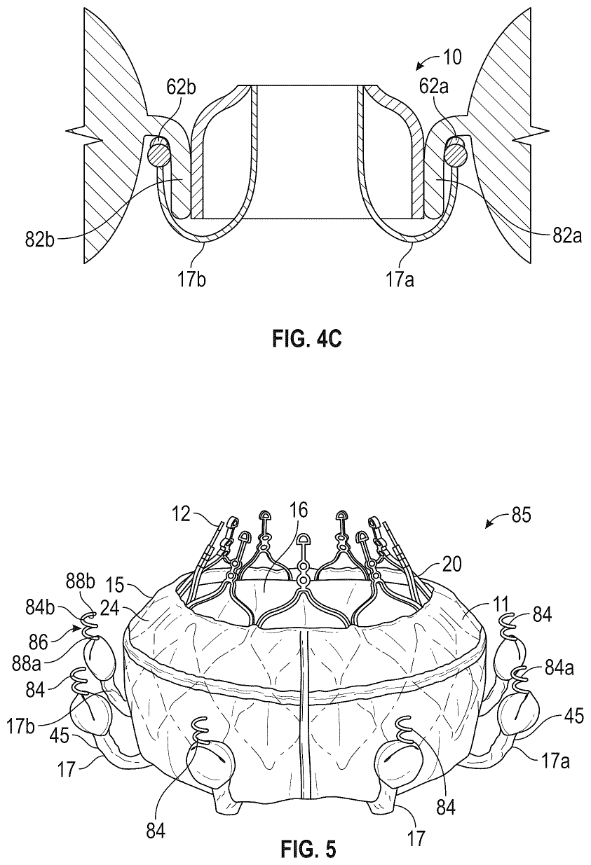

Anchors 17 are shown in FIGS. 1A-2 in a deployed or expanded configuration, in which the tips of the anchors 17 extend proximally. The anchors 17 are adapted to be in undeployed, unexpanded, or linearized configurations wherein the tips of the anchors 17 extend distally. Such a configuration is shown in FIG. 4A. The anchors 17 are adapted to be flexible. Upon deployment, the anchors 17 can be configured to move from the undeployed configuration radially outward to the deployed configuration, with the tips flipped towards the proximal direction. Such an operation allows the anchors 17 to flip over the native valve leaflets to anchor to the native valve leaflets during deployment. Such a configuration is shown in FIG. 4C for example. Other deployment methods for the anchors 17 can be utilized in examples as desired.

Referring to FIG. 2, the proximal portion of the inner frame 18 is coupled to a proximal portion of the plurality of prosthetic valve leaflets 16. The inner frame 18 supports the prosthetic valve leaflets 16. The prosthetic valve leaflets 16 can be coupled to the inner frame 18 and extend radially inward from the inner frame 18. The prosthetic valve leaflets 16 couple to the inner frame 18 via an intermediate body 28 that supports the prosthetic valve leaflets 16 and couples the leaflets 16 to the inner frame 18 via sutures or another method as desired.

The prosthetic valve leaflets 16 surround a flow channel 27 as marked in FIG. 2 and can move between open and closed states to control flow through the flow channel 27. As shown in FIG. 2, the proximal end of the prosthetic valve 10 comprises an inflow end of the valve 10, and the distal end of the prosthetic valve 10 comprises an outflow end, although other configurations can be utilized as desired. The prosthetic valve leaflets 16 are positioned around a central axis 61 of the prosthetic valve 10. The inner frame 18 and outer frame 20 each surround the central axis 61 of the prosthetic valve 10.

The anchors 17 can each extend radially outward from the flow channel 27 and radially outward from the prosthetic valve leaflets 16 of the valve 10. The anchors 17 can be adapted to extend radially outward from the inner valve frame 18 and across the gap 39 to the tip of the respective anchor 17. The anchors 17 are coupled to the distal portion of the inner frame 18. The anchors 17 can each include a proximal portion 29 and a distal portion 45, with the proximal portion 29 coupled to the inner frame 18 and the distal portion 45 comprising a tip of the respective anchor 17. The anchors 17 extend vertically from the proximal portion 29 to the tip at the distal portion 45 when the valve 10 is deployed.

An indicator can be provided for indicating capture of a native valve leaflet and/or proper placement of the one or more of the anchors 17. An indicator may take a variety of forms while remaining within the scope of the invention.

For example, referring to FIG. 1A, a conformable indicator 62 is provided for indicating capture of a native valve leaflet under imaging. An appearance of the indicator 62 can vary in shape or brightness under imaging for confirming the proper placement of the anchor. The indicator 62 may take the form of exemplary indicators 62a, 62b marked in FIG. 1A for identification purposes.

The indicator 62 varies in appearance under ultrasound imaging depending on the location of the indicator and/or forces acting upon it. For example, the indicator 62 has a visibility that is reduced under ultrasound imaging to indicate proper placement of the anchor and capture of the native valve leaflet. The indicator comprises an echogenic marker that has an altered appearance under ultrasound imaging when the indicator contacts native tissue. For example, an indicator on an anchor changes appearance when the anchor is properly positioned radially outward of (e.g., on the ventricular side of) native valve leaflets. When properly positioned, the indicator contacts native tissue, such as the leaflet and/or annulus, which confirms that the anchor is properly positioned.

The indicator 62 can be positioned in a variety of locations, depending on the desired information required for confirming proper placement. For example, referring to FIG. 1A, the indicator 62 is positioned on one or more of the anchors 17. As shown in FIG. 1A, each anchor 17 includes one indicator 62, although other configurations can be utilized as desired (e.g., only one of the anchors 17 includes an indicator 62, or at least one of the anchors 17 includes an indicator 62). The indicators 62a, 62b are marked in FIG. 1A upon exemplary anchors 17a, b. In examples, the indicator 62 is positioned on a distal portion 45 or tip of a respective anchor 17. As such, the indicator 62 is positioned radially outward of a native valve leaflet when the native leaflet is captured between the anchor and the sealing body 11. When the anchor is properly seated, the indicator abuts against native tissue (e.g., the ventricular side of native annulus) and therefore deforms. If an anchor 17 fails to capture or mis-captures a native valve leaflet, then the indicator 62 will be positioned radially inward of the native valve leaflet, wherein the indicator is not compressed against native tissue (e.g., is not contacting the annulus). Accordingly, the indicator 62 provides visual feedback information regarding anchor placement, which can be seen using visualization techniques described herein. When properly placed, the indicator on the anchor changes shape due to contact with native tissue. In contrast, the indicator 62 has a substantially unaltered appearance when the anchor is not properly placed and the leaflet is not captured, which alerts the physician to a potential problem.

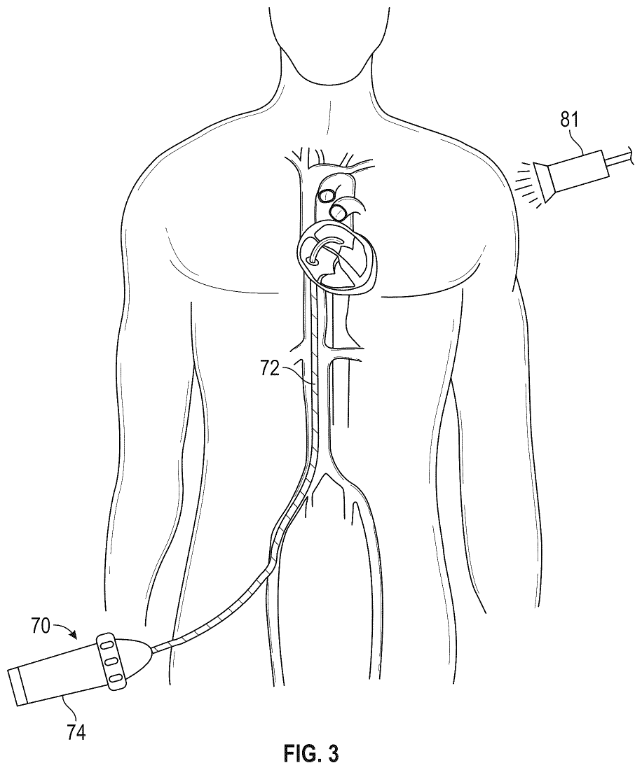

Referring to FIG. 3, the prosthetic valve 10 is deployed to an implantation site via a delivery system 70. FIG. 3 illustrates advancement of a delivery system 70 for deployment of the prosthetic valve 10 to an implantation site. The delivery system 70 includes an elongate shaft 72 having a proximal portion and a distal portion, with the proximal portion coupled to a housing in the form of a handle 74. The delivery system 70 is advanced through the vasculature of a patient, which can include the femoral vein as shown in FIG. 3. Other entries can be utilized in examples, including transapical, or via surgical methods such as thoracotomy or open-heart surgery.

The prosthetic valve 10 is positioned within an implant retention area of the delivery system 70. The prosthetic valve 10 can be covered with a capsule, for example, or can otherwise be retained prior to deployment. The prosthetic valve 10 can be deployed as a self-expanding prosthetic valve or can be a balloon-expandable prosthetic valve (e.g., positioned upon an inflatable balloon upon entry into the patient's body, or slid onto an inflatable balloon within the patient's body). The prosthetic valve can also be mechanically expanded, among other forms of deployment.

The delivery system 70 is advanced to pass into an atrium of a heart and can pass transeptally into another atrium (e.g., from the right atrium to the left atrium) to reach an implantation site. Such a delivery approach can be utilized for mitral native valve access for example. In examples, the delivery system 70 extends to the right atrium for tricuspid access, or other delivery approaches to other implantation sites can be utilized in examples as desired.

FIG. 4A illustrates a side cross-sectional view of the prosthetic valve 10 in a compressed configuration and protruding slightly from a capsule 79 of the delivery system 70. The anchors 17a, b are shown to extend longitudinally in an elongated configuration. The prosthetic valve 10 is positioned to be deployed to a native valve 80.

Referring to FIG. 3, upon deployment of the prosthetic valve 10 from the delivery system, the implantation site can be imaged. The imaging may take a variety of forms. As shown in FIG. 3, the imaging can comprise ultrasound imaging that can be produced by an echocardiograph device 81. The echocardiograph device 81 can comprise a transducer or other form of echocardiograph device 81 that produces images via ultrasound waves. In examples, the echocardiograph device 81 is positioned external of the patient's body. However, in examples an echocardiograph device is adapted to be positioned within the patient's vasculature for imaging. Other forms of imaging such as fluoroscopy or other forms can be utilized as desired. Combinations of forms of imaging, such as a combination of ultrasound and fluoroscopy can be utilized. Combinations of other forms of imaging can be utilized as well.

The imaging allows a user (e.g., a surgeon or other form of medical technician) to view the implantation site during an implantation procedure.

Imaging the implantation site beneficially allows the user to determine a desired placement of the prosthetic valve 10 and a desired anchoring of one or more of the anchors 17. For example, capture of a native valve leaflet by one or more of the anchors 17 can beneficially be imaged. A user can beneficially determine if the anchors are placed properly and capture of the native valve leaflets has occurred, which identifies whether a proper implantation of the prosthetic valve 10 has occurred.

Referring to FIGS. 4A and 4B, indicators 62a, b are adapted to indicate capture of a native valve leaflet 82a, b by the one or more anchors 17a, b. For example, referring to FIG. 4A, the anchors 17a, b include the indicators 62a, b, which are positioned on the tips of the respective anchors 17a, b. The indicators 62a, b have an appearance under imaging, and particularly under ultrasound imaging. For example, the indicators 62a, b have a brightness under ultrasound imaging as the anchors 17a, b are deployed partially from the capsule 79 as shown in FIG. 4A.

Upon the anchors 17a, b being fully deployed as shown in FIG. 4B, an anchor 17a misses capture of a leaflet 82a as shown on the right side of FIG. 4B. As such, an indicator 62a remains uncovered by the leaflet 82a and has an appearance under imaging, which can be the same appearance as in the position shown in FIG. 4A. For example, the brightness of the indicator 62a for the anchor 17a that missed capture of the leaflet 82a has a same brightness as in the position shown in FIG. 4A.

The indicator 62b for the anchor 17b that captured the leaflet 82b (shown on the left side of FIG. 4B), however, has a varied appearance from the appearance in the position shown in FIG. 4A. The appearance of the indicator 62b for the anchor 17b that captured the leaflet 82b varies from the appearance of the indicator 62a for the anchor 17a that missed capture of the leaflet 82a. For example, the indicator 62b for the anchor 17b that captured the leaflet 82b has a darker appearance under imaging than the appearance of the indicator 62a for the anchor 17a that missed capture of the leaflet 82a. Based on the appearance of the indicator 62a, b, a user is able to determine if missed capture of the leaflet 82a has occurred.

In examples, the indicators 62a, b can be imaged from the atrium such that the tissue of the native valve leaflet 82b covers the indicator 62b to reduce the brightness of such an indicator 62b. The layer of leaflet tissue covering the indicator 62b, as opposed to the lack of tissue covering the indicator 62a, can produce a darker appearance of the indicator 62b and an indication that the leaflet 82b has been captured. The brighter appearance of the indicator 62a indicates that the leaflets 82a has not been captured. Other differences in the appearance of the indicators 62a, b may result.

The user, in examples, is able to redeploy an anchor 17a or attempt to recapture the leaflet 82a for proper deployment of the prosthetic valve 10. The user can perform a redeployment or recapture procedure to capture the leaflet 82a. The user visualizes the indicator 62a to determine if capture of the leaflet 82a has occurred.

FIG. 4C, for example, illustrates a view of the prosthetic valve 10 with both leaflets 82a, b captured. The indicators 62a, b have a similar appearance as each other, with reduced visibility to indicate capture of the respective leaflet 82a, b. A user is able to determine that both leaflets 82a, b have been captured based on the appearance of the indicators 62a, b. The imaging can occur from the atrium or from another location as desired.

The features of FIGS. 1 through 4C may be utilized solely or in combination with any example disclosed herein.

In examples, the configuration of one or more of the indicators may be varied as desired. FIG. 5, for example, illustrates an indicator 84 that is adapted to move to indicate capture of a native valve leaflet. The indicator 84 is adapted to indicate capture of a native valve leaflet under imaging. The appearance of the indicator 84 under imaging can vary to indicate capture of the native valve leaflet. The indicator 84 include exemplary indicators 84a, b marked in FIG. 5 for identification purposes.

Each indicator 84 comprises an elongate body in examples. A plurality of elongate bodies are provided. Each elongate body is positioned on a respective one of the anchors 17. The elongate body, for example, is positioned on a distal portion 45 or distal tip of the respective anchor 17. The elongate body protrudes proximally from the distal portion 45 or distal tip of the respective anchor 17 as shown in FIG. 5. Indicators 84a, b are positioned upon respective anchors 17a, b.

Each elongate body is configured as a spring in examples. The spring comprises a helical spring having a coil shape as shown in FIG. 5. As such, the spring is set in an extended position, which can be compressed to a compressed position upon distal pressure being applied to the spring. Upon release of the pressure, the spring is biased to extend back to the extended position. In examples, the configuration of the elongate bodies can be varied as desired.

The difference in appearance of the elongate body between the extended position and the compressed position is able to be viewed under imaging. For example, the spacing 86 (marked in FIG. 5) between adjacent wraps 88a, 88b of the elongate body is visible under imaging with the elongate body in the extended position. A reduced spacing 86 between the wraps 88a, 88b can further be visible under imaging with the elongate body compressed.

A user can view the appearance of the indicator 84 to determine if a force has been applied to the indicator 84, to determine if capture of a native valve leaflet has occurred.

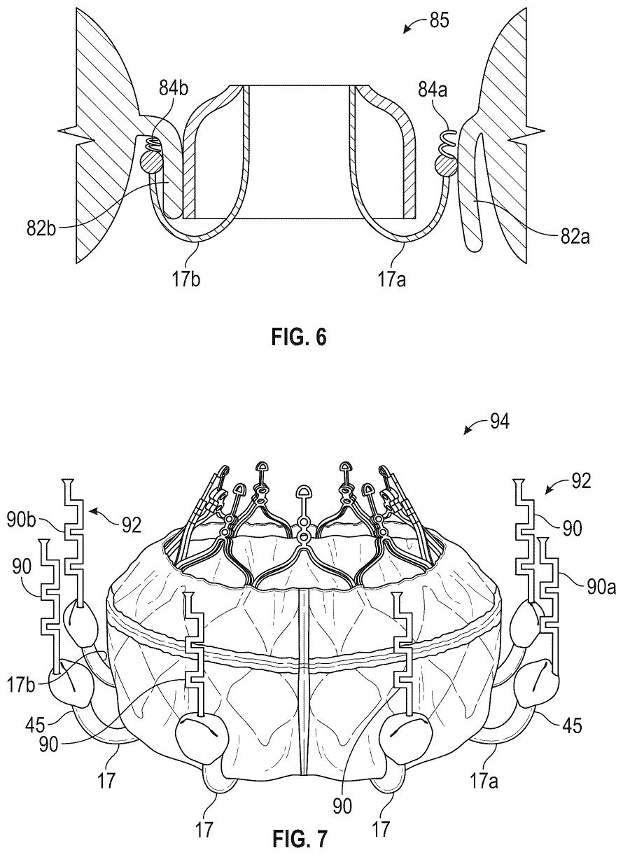

For example, referring to FIG. 6, deployment of the prosthetic valve 85 has occurred. An anchor 17a (shown to the right of the page in FIG. 6) has missed capture of the leaflet 82a. The indicator 84a upon such anchor 17a accordingly remains in an extended position, which can be visible via imaging. The imaging comprises fluoroscopy or ultrasound, or combinations of fluoroscopy and ultrasound, or other forms of imaging. A user accordingly determines that a failed capture or missed capture of the leaflet 82a has occurred by viewing an extended position of the indicator 84a.

An anchor 17b (shown to the left of the page in FIG. 6) has made capture of the leaflet 82b. The indicator 84b upon such anchor 17b accordingly moves to a compressed position, which can be visible via the imaging. A user accordingly determines that capture of the leaflet 82b has occurred by viewing a compressed position of the indicator 84b.

In examples, a user determines if capture of a leaflet has occurred by viewing a difference in position between the indicators 84a, b. For example, if the indicator 84a shown to the right of the page in FIG. 6 has a greater height than the indicator 84b shown to the left of the page in FIG. 6, then a user can determine that a missed capture has occurred.

The features of FIGS. 5 and 6 may be utilized solely or in combination with any example disclosed herein.

In examples, the configuration of the one or more indicators can be varied as desired. FIG. 7, for example, illustrates an indicator 90 that is adapted to move to indicate capture of a native valve leaflet. The indicator 90 can be adapted to indicate capture of a native valve leaflet under imaging. The appearance of the indicator 90 under imaging can vary to indicate capture of the native valve leaflet. The indicators 90 include exemplary indicators 90a, b marked in FIG. 7 for identification purposes.

The indicator 90 comprises an elongate body. A plurality of elongate bodies can be utilized in examples. Each elongate body is positioned on a respective one of the anchors 17. The elongate body, for example, is positioned on a distal portion 45 or distal tip of the respective anchor 17. The elongate body protrudes proximally from the distal portion 45 or distal tip of the respective anchor 17 as shown in FIG. 7. Indicators 90a, b are positioned upon respective anchors 17a, b.

Each elongate body is configured as a spring in examples. The spring comprises an elongate lever arm adapted to be deflected laterally. The spring includes an undulating shape to increase the flexibility of the spring. In examples, the spring is in a straightened or elongated position as shown in FIG. 7. In examples, a portion of the spring such as a tip portion 92 is adapted to deflect laterally to move to a deflected position. Such deflection can be visible under imaging to allow a user to determine a position of the anchors 17 and if capture of a leaflet has occurred.

A user views the appearance of the indicator 90 to determine if a force has been applied to the indicator 90, to determine a position of the anchors 17 and if capture of a leaflet has occurred.

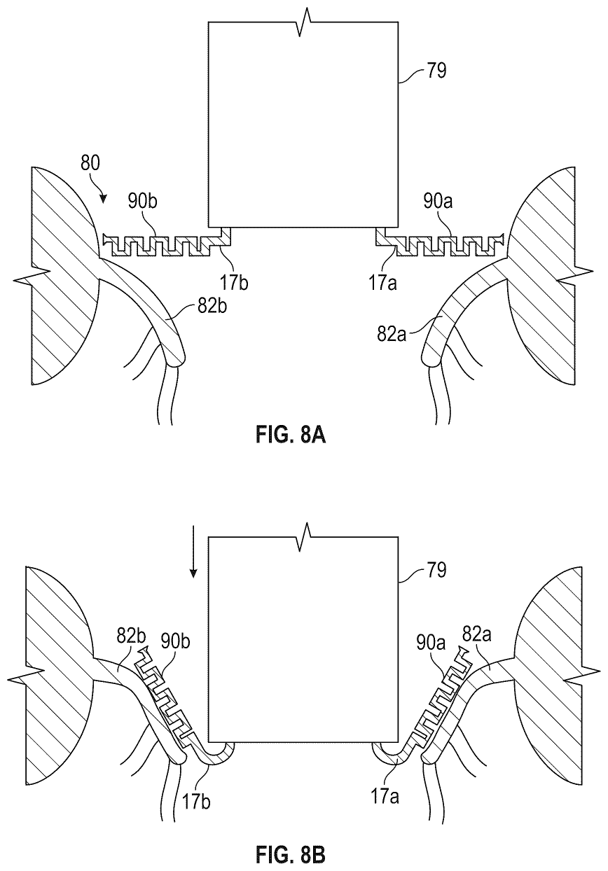

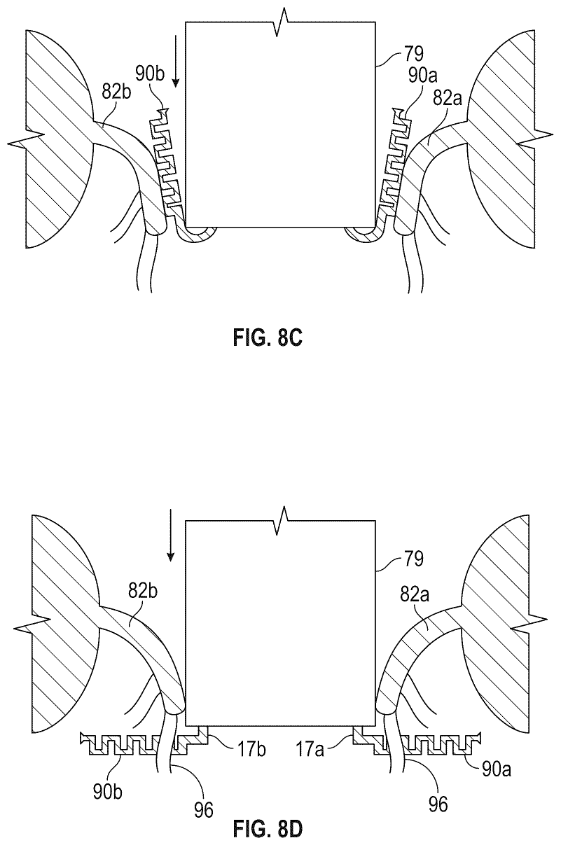

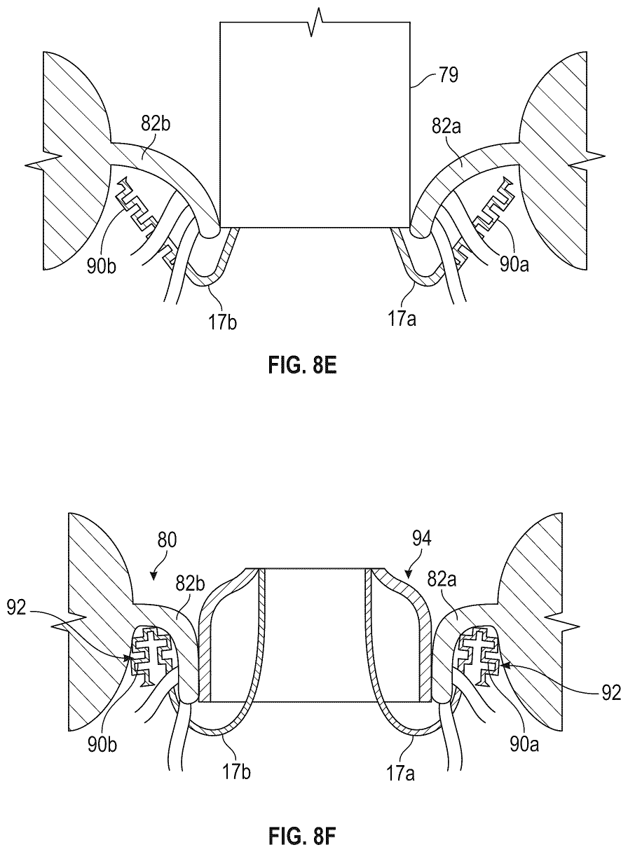

For example, referring to FIG. 8A, partial deployment of anchors 17a, b has occurred. The indicators 90a, b extend from the capsule 79 and extend laterally outward from the capsule 79. The indicators 90a, b can extend perpendicular with respect to the capsule 79 or can extend at another angle as desired.

The indicators 90a, b extend outward from the capsule 79 at a length that allows the indicators 90a, b to contact leaflets 82a, b of the native valve 80 or another portion of the native valve 80 (e.g., the annulus) upon extension of the indicators 90a, b. The indicators 90a, b protrude radially outward from the capsule 79 and are spaced circumferentially from each other such that the indicators 90a, b contact a portion of the native valve 80 at a plurality of circumferential positions.

A user views the position of the indicators 90a, b via imaging. The imaging can comprise fluoroscopy or ultrasound, or combinations of fluoroscopy and ultrasound, or other forms of imaging. A user accordingly visualizes a position of the indicators 90a, b. For example, referring to FIG. 8A, a user visualizes that the indicators 90a, b are straightened and extend laterally from the capsule 79. As such, a user determines that the indicators 90a, b have not yet contacted the leaflets 82a, b and thus are positioned axially offset from the annulus of the native valve 80. One or more of the indicators 90a, b can be deflected to indicate a variation in the position of the indicators 90a, b and the prosthetic valve 94.

The indicators 90a, b are advanced distally as part of a deployment procedure. For example, referring to FIG. 8B, the indicators 90a, b can be advanced distally, which can occur along with a distal movement of the capsule 79. The indicators 90a, b can deflect proximally and can deflect radially inward towards the capsule 79. The deflected position of the indicators 90a, b is shown in FIG. 8B. A user can image the position of the indicators 90a, b to determine that the indicators 90a, b and accordingly the prosthetic valve 94 (marked in FIG. 7) are now positioned between the leaflets 82a, b. The user can further determine that the indicators 90a, b have not advanced distal of the leaflets 82a, b because the indicators 90a, b are in the deflected position.

A user views a change in the amount of deflection of the indicators 90a, b to determine a position of the prosthetic valve 94 and the anchors 17a, b. For example, referring to FIG. 8C, a user visualizes that the indicators 90a, b have been advanced further distal by viewing an increased deflection of the indicators 90a, b radially inward. A user determines that the indicators 90a, b have not advanced distal of the leaflets 82a, b because the indicators 90a, b are in the deflected position.

Referring to FIG. 8D, the indicators 90a, b can be advanced further distal to extend laterally outward from the capsule 79 in a straightened or elongated position. The straightened configuration of the indicators 90a, b indicates that the indicators 90a, b are properly threaded through chordae 96 of the heart in order to hook around the leaflets 82a, b. Thus, a user determines that the anchors 17a, b are in position to hook around the leaflets 82a, b. If one of the indicators 90a were in a straightened position and another indicator 90b was visualized to be in a deflected position, then a user determines that an indicator 90b was not in position to hook around a leaflet and attempts to reposition the indicator 90b and the associated anchor 17b.

With the indicators 90a, b indicating proper threading through chordae 96 of the heart in order to hook around the leaflets 82a, b, the anchors 17a, b continue to be deployed to hook around the leaflets 82a, b as shown in FIG. 8E for example.

The indicators 90a, b have sufficient length such that the tip portions 92 are moved into the deflected position upon full deployment of the prosthetic valve 94. The tip portions 92, for example, contact a portion of the native valve 80 comprising the annulus or another portion of the native valve 80 (e.g., the leaflets 82a, b) to indicate that capture of the native valve leaflets 82a, b has occurred. FIG. 8F, for example, illustrates such a configuration. In an example in which the capture has not occurred, then the tip portions 92 would not be deflected. A user determines if a failed capture or mis-capture of the leaflet 82a, b has occurred due to the tip portion 92 remaining in a straightened position with respect to the remainder of the indicator 90a, b.

The deployment sequence can be varied from the sequence shown in FIGS. 8A-8F as desired.

The features of FIGS. 7-8F may be utilized solely or in combination with any example disclosed herein.

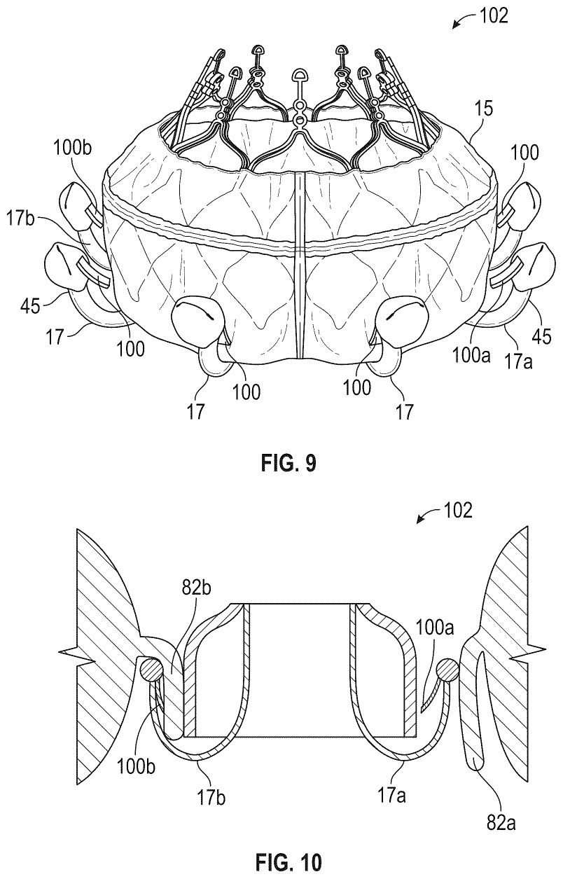

In examples, the configuration of the indicators can be varied as desired. FIG. 9, for example, illustrates an indicator 100 that is adapted to move to indicate capture of a native valve leaflet. The indicator 100 is adapted to indicate capture of a native valve leaflet under imaging. The appearance of the indicator 100 under imaging varies to indicate capture of the native valve leaflet. The indicators 100 include exemplary indicators 100a, b marked in FIG. 9 for identification purposes.

The indicator 100 comprises an elongate body. A plurality of elongate bodies can be provided in examples. Each elongate body is positioned on a respective one of the anchors 17. The elongate body, for example, can be positioned on a distal portion 45 or distal tip of the respective anchor 17. The elongate body protrudes distally from the distal portion 45 or distal tip of the respective anchor 17 as shown in FIG. 9. Indicators 100a, b are positioned upon respective anchors 17a, b.

Each elongate body is adapted as a spring in examples. The spring comprises an elongate lever arm adapted to be deflected radially outward, toward the anchor 17. The spring is configured as a flat spring or cantilever spring with the fixed end of the spring coupled to the anchor 17 and the free end extending radially inward towards the valve body 15. A deflection of the spring to a deflected position is visible under imaging to allow a user to determine if capture of a leaflet has occurred.

A user views the appearance of the indicator 100 to determine if a force has been applied to the indicator 100, to determine if capture of a native valve leaflet has occurred.

For example, referring to FIG. 10, deployment of the prosthetic valve 102 has occurred. An anchor 17a (shown to the right of the page in FIG. 10) has missed capture of the leaflet 82a. The indicator 100a upon such anchor 17a accordingly remains in an undeflected position or extends radially inward, which can be visible via imaging. The imaging can comprise fluoroscopy or ultrasound, or combinations of fluoroscopy and ultrasound, or other forms of imaging. A user accordingly can determine that a failed capture or missed capture of the leaflet 82a has occurred by viewing the undeflected position of the indicator 100a.

An anchor 17b (shown to the left of the page in FIG. 10) has made capture of the leaflet 82b. The indicator 100b upon such anchor 17b accordingly can move to a deflected position, which can be visible via the imaging. A user accordingly determines that capture of the leaflet 82b has occurred by viewing a deflected position of the indicator 100b.

In examples, a user determines if capture of a leaflet has occurred by viewing a difference in position between the indicators 100a, b. For example, if the indicator 100a shown to the right of the page in FIG. 10 has a lesser deflection than the indicator 100b shown to the left of the page in FIG. 10, then a user can determine that a missed capture has occurred.

The features of FIGS. 9-10 may be utilized solely or in combination with any example disclosed herein.

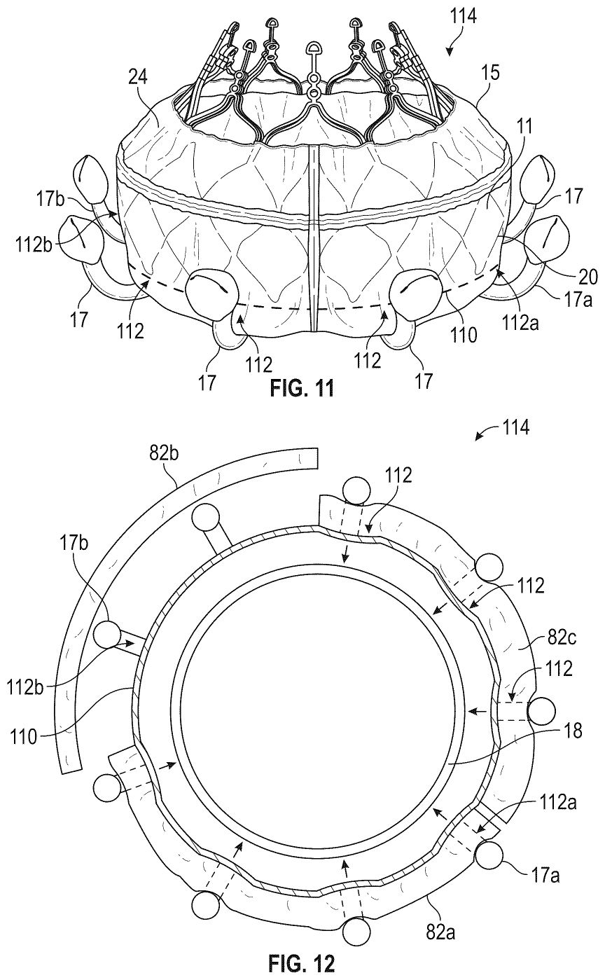

In examples, the configuration of the indicators can be varied as desired. FIG. 11, for example, illustrates an indicator 110 that is adapted to move to indicate capture of a native valve leaflet. The indicator 110 is adapted to indicate capture of a native valve leaflet under imaging. The appearance of the indicator 110 under imaging varies to indicate capture of the native valve leaflet.

The indicator 110 comprises an elongate body. The elongate body is positioned on the valve body 15 and comprises a ring extending circumferentially about the valve body 15. The ring comprises a full ring (a continuous body around the entirety of the valve body 15) or a partial ring (extending partially about the valve body 15). The ring is positioned radially inward of the anchors 17. The ring is positioned at an axial height on the valve body 15 that positions the ring opposite the position of the anchors 17. As such, a native heart valve leaflet will be positioned between the ring and a respective anchor 17 upon capture of the leaflet by the anchor 17. The indicator 110 comprises a radiopaque ring in examples.

The indicator 110 is positioned on the outer frame 20 or sealing body 11 of the valve body 15. In examples, the indicator 110 is positioned on the sealing skirt 24 of the sealing body 11. For example, the indicator 110 can be stitched into the sealing skirt 24 or otherwise coupled to the sealing skirt 24. In examples, the indicator 110 is positioned on the sealing skirt 24 distal of the outer frame 20 to allow the indicator 110 to more easily deflect inward upon capture of a native valve leaflet.

The indicator 110 comprises a flexible material that is adapted to deflect. The flexible material is adapted to deflect radially inward upon an inward force being applied to the flexible material. For example, upon capture of a native heart valve leaflet between an anchor 17 and the indicator 110, the indicator 110 deflects inward due to the force applied by the native heart valve leaflet. The inward deflection of the indicator 110 can be localized in examples. The indicator 110 can be adapted to deflect radially inward at portions 112 of the indicator 110 that can be positioned opposed to the respective anchors 17.

A user views the appearance of an indicator 110 to determine if a force has been applied to the indicator 110, to determine if capture of a native valve leaflet has occurred.

For example, referring to FIG. 12, deployment of the prosthetic valve 114 has occurred. A top view or axial view of the prosthetic valve 114 is provided. Certain anchors (for example, anchor 17a) have captured a leaflet 82a (and leaflet 82c). Other anchors (for example, anchor 17b) have failed to capture or mis-captured the leaflet 82b.

The indicator 110 is imaged to determine if capture of the leaflets 82a-c has occurred. The imaging can comprise fluoroscopy or ultrasound, or combinations of fluoroscopy and ultrasound, or other forms of imaging. The imaging occurs in an axial dimension of the prosthetic valve 114, as represented in FIG. 12.

A user determines if deflection of the indicator 110 occurs to determine if capture of the leaflets 82a-c has occurred. A portion 112a of the indicator 110 is positioned opposite the anchor 17a. A portion 112b of the indicator 110 is positioned opposite the anchor 17b. For example, a user determines that inward deflection of the indicator 110 at the portion 112a has occurred, to determine that the leaflet 82a has been captured and accordingly has pressed the indicator 110 radially inward. A user determines that a lack of deflection or a lesser deflection of a portion 112b of the indicator 110 has occurred, to determine that a failed capture or mis-capture of the leaflet 82b has occurred.

In examples, a user determines if capture of a leaflet has occurred by viewing a difference in position between the portions 112a, 112b of the indicator 110. For example, if the portion 112b of the indicator 110 has a lesser deflection than the portion 112a, then a user determines that a missed capture has occurred at the portion 112b. If the indicator 110 is deflected at each of the anchor locations, then a user determines that the leaflets have each been captured by the anchors 17.

The features of FIGS. 11 and 12 may be utilized solely or in combination with any example disclosed herein.

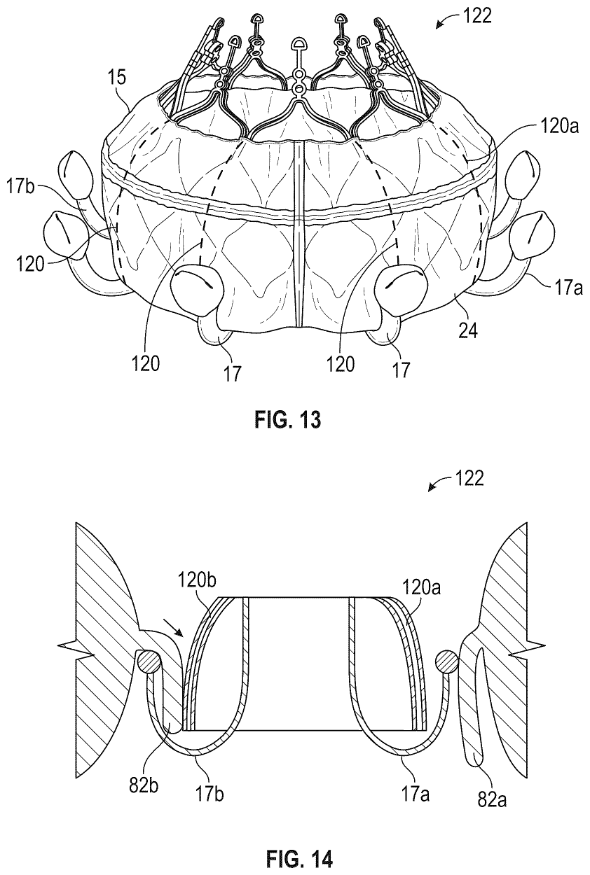

In examples, the configuration of the indicators can be varied as desired. FIG. 13, for example, illustrates an indicator 120 that is adapted to move to indicate capture of a native valve leaflet. The indicator 120 can be adapted to indicate capture of a native valve leaflet under imaging. The appearance of the indicator 120 under imaging varies to indicate capture of the native valve leaflet.

The indicator 120 comprises an elongate body. The elongate body is positioned on the valve body 15 and extends axially along the valve body 15. The elongate body comprises a wire that extends axially along the valve body 15.

The wire is positioned radially inward of one or more of the anchors 17. The wire is positioned at a circumferential position on the valve body 15 that positions the wire opposite a respective one of the anchors 17. As such, a native heart valve leaflet will be positioned between the wire and a respective anchor 17 upon capture of the leaflet by the anchor 17.

In examples, the indicator 120 is positioned on the outer frame 20 or sealing body 11 of the valve body 15. In examples, the indicator 120 is positioned on the sealing skirt 24 of the sealing body 11. For example, the indicator 120 can be stitched into the sealing skirt 24 or otherwise coupled to the sealing skirt 24.

The indicator 120 comprises a flexible material that is adapted to deflect. The flexible material is adapted to deflect radially inward upon an inward force being applied to the flexible material. For example, upon capture of a native heart valve leaflet between an anchor 17 and the indicator 120, the indicator 120 deflects inward due to the force applied by the native heart valve leaflet.

A user views the appearance of an indicator 120 to determine if a force has been applied to the indicator 120, to determine if capture of a native valve leaflet has occurred. In examples, a plurality of indicators 120 are utilized. The plurality of indicators 120 are spaced circumferentially from each other, each at a position of a respective one of the anchors 17. Each indicator 120 extends axially along the valve body 15.

For example, referring to FIG. 14, deployment of the prosthetic valve 122 has occurred. A side view or lateral view of the prosthetic valve 122 is provided. Certain anchors (for example, anchor 17a) have failed to capture or mis-captured the leaflet 82a. Other anchors (for example, anchor 17b) have captured the leaflet 82b.

The indicators 120 are imaged to determine if capture of the leaflets 82a, b has occurred. The imaging can comprise fluoroscopy or ultrasound, or combinations of fluoroscopy and ultrasound, or other forms of imaging. The imaging occurs in a lateral dimension of the prosthetic valve 122, as represented in FIG. 14.

The indicators 120 include exemplary indicators 120a, b marked in FIG. 14 for identification purposes.

A user determines if deflection of the indicators 120a, b occurs to determine if capture of the leaflets 82a, b has occurred. For example, a user determines that inward deflection of the indicator 120b has occurred, to determine that the leaflet 82b has been captured and accordingly has pressed the indicator 120b radially inward. A user determines that a lack of deflection or a lesser deflection of the indicator 120a has occurred, to determine that a failed capture or mis-capture of the leaflet 82a has occurred.

In examples, a user determines if capture of a leaflet has occurred by viewing a difference in position between the indicators 120a, b. For example, if the indicator 120a has a lesser deflection than the indicator 120b, then a user determines that a missed capture has occurred at the indicator 120a. If the indicators 120a, b is each deflected at each of the anchor locations then a user determines that the leaflets 82a, b have each been captured by the anchors 17a, b.

The features of FIGS. 13 and 14 can be utilized solely or in combination with any example disclosed herein.

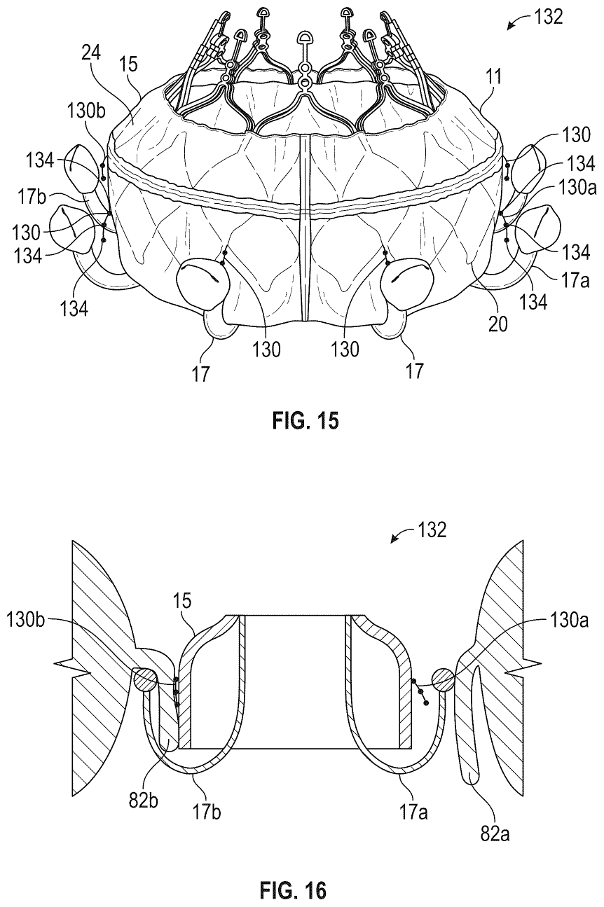

In examples, the configuration of the indicators can be varied as desired. FIG. 15, for example, illustrates an indicator 130 that is adapted to move to indicate capture of a native valve leaflet. The indicator 130 is adapted to indicate capture of a native valve leaflet under imaging. The appearance of the indicator 130 under imaging varies to indicate capture of the native valve leaflet.

The indicator 130 comprises an elongate body. A plurality of elongate bodies is utilized in examples. Each elongate body is positioned on the valve body 15. The elongate body is positioned on the outer frame 20 or sealing body 11 of the valve body 15. In examples, the indicator 130 is positioned on the sealing skirt 24 of the sealing body 11. The elongate body protrudes distally from the valve body 15 as shown in FIG. 15.

Each elongate body is configured as a spring in examples. The spring comprises an elongate lever arm adapted to be deflected radially inward, toward the valve body 15. The spring is configured as a flat spring or cantilever spring with the fixed end of the spring coupled to the valve body and the free end extending radially outward towards a respective one of the anchors 17. A deflection of the spring to a deflected position can be visible under imaging to allow a user to determine if capture of a leaflet has occurred.

The indicators 130 include exemplary indicators 130a, b marked in FIG. 15 for identification purposes.

A user views the appearance of the indicator 130 to determine if a force has been applied to the indicator 130, to determine if capture of a native valve leaflet has occurred.

For example, referring to FIG. 16, deployment of the prosthetic valve 132 has occurred. An anchor 17a (shown to the right of the page in FIG. 16) has missed capture of the leaflet 82a. The indicator 130a positioned proximate such anchor 17a accordingly remains in an undeflected position or extends radially outward from the valve body 15, which is visible via imaging. The imaging can comprise fluoroscopy or ultrasound, or combinations of fluoroscopy and ultrasound, or other forms of imaging. A user accordingly determines that a failed capture or missed capture of the leaflet 82a has occurred by viewing the undeflected position of the indicator 130a.

An anchor 17b (shown to the left of the page in FIG. 16) has made capture of the leaflet 82b. The indicator 130b proximate such anchor 17b accordingly moves to a deflected position, which is visible via the imaging. A user accordingly determines that capture of the leaflet 82b has occurred by viewing a deflected position of the indicator 130b.

In examples, each indicator 130 includes one or more markers that improve ease of imaging of the indicator 130. For example, one or more marker beads 134 are provided for each indicator 130. Each marker bead 134 is spaced from another marker bead 134 on the indicator 130 and indicates a position of the portion of the indicator 130. In examples, the marker beads 134 are configured to be visible under imaging such as fluoroscopy.

In examples, a user determines if capture of a leaflet has occurred by viewing a difference in position between the indicators 130. For example, if the indicator 130a shown to the right of the page in FIG. 16 has a lesser deflection than the indicator 130b shown to the left of the page in FIG. 16, then a user determines that a missed capture has occurred.

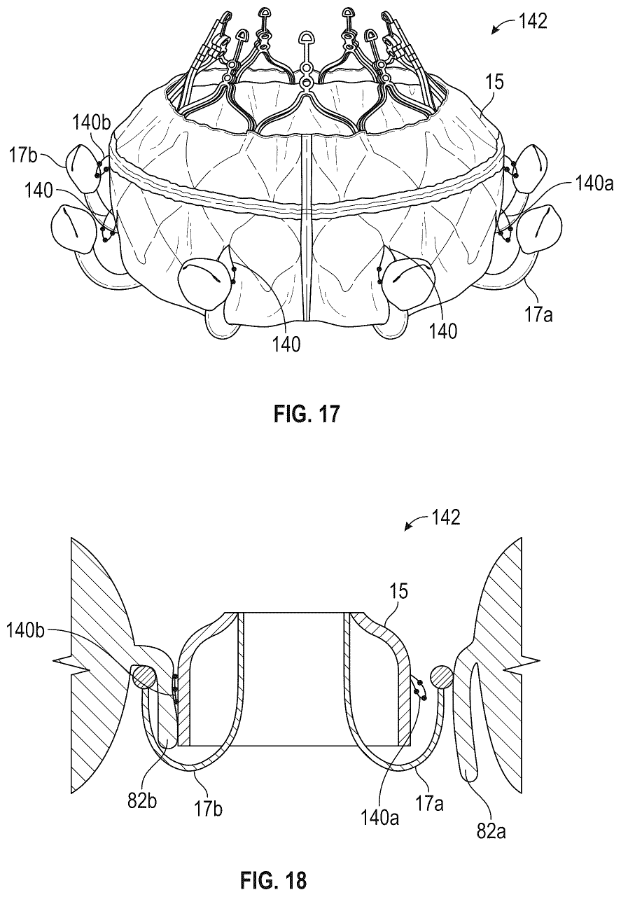

In examples, the configuration of the indicators is varied as desired. FIG. 17, for example, illustrates an indicator 140 that is configured similarly as the indicators 130 shown in FIGS. 15 and 16, yet formed into a loop. Each end of the loop is coupled to the valve body 15 in examples. As such, a reduced possibility of puncture of portion of the native heart valve with an indicator 140 occurs because each end of the loop is coupled to the valve body 15.

The indicators 140 include exemplary indicators 140a, b marked in FIG. 17 for identification purposes.

The indicators 140 can include one or more markers, which may be similar to the configuration of the markers discussed with respect to FIGS. 15 and 16.

The indicators 140 can be imaged by a user in a similar manner as discussed with regard to the indicators 130 shown in FIGS. 15 and 16.

For example, referring to FIG. 18, deployment of the prosthetic valve 142 has occurred. An anchor 17a (shown to the right of the page in FIG. 18) has missed capture of the leaflet 82a. The indicator 140a positioned proximate such anchor 17a accordingly remains in an undeflected position or extends radially outward from the valve body 15, which is visible via imaging. The imaging comprises fluoroscopy or ultrasound, or combinations of fluoroscopy and ultrasound, or other forms of imaging. A user accordingly determines that a failed capture or missed capture of the leaflet 82a has occurred by viewing the undeflected position of the indicator 140a.

An anchor 17b (shown to the left of the page in FIG. 18) has made capture of the leaflet 82b. The indicator 140b proximate such anchor 17b accordingly moves to a deflected position, which is visible via the imaging. A user accordingly determines that capture of the leaflet 82b has occurred by viewing a deflected position of the indicator 140b.

In examples, a user determines if capture of a leaflet has occurred by viewing a difference in position between the indicators 140a, b. For example, if the indicator 140a shown to the right of the page in FIG. 18 has a lesser deflection than the indicator 140b shown to the left of the page in FIG. 18, then a user determines that a missed capture has occurred.

The features of FIGS. 15-18 may be utilized solely or in combination with any example disclosed herein.

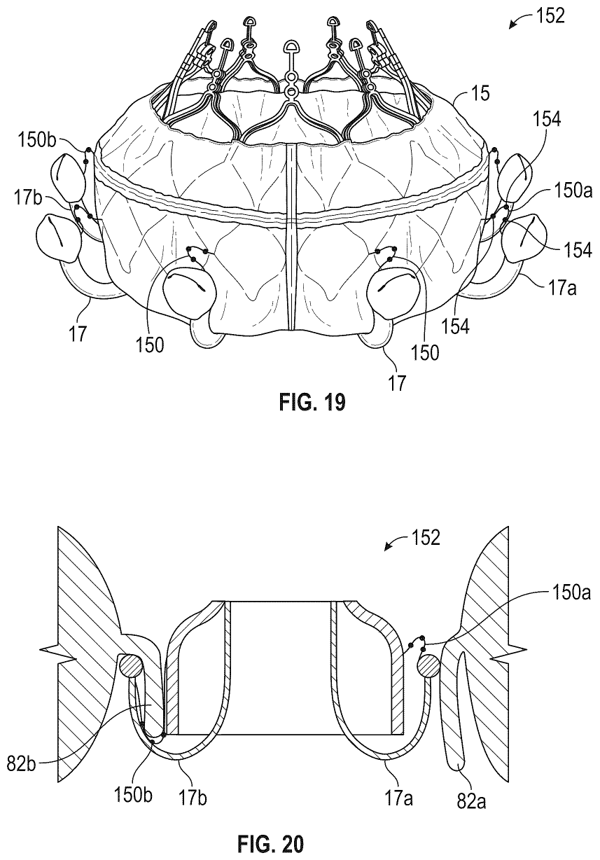

In examples, the configuration of the indicators may be varied as desired. FIG. 19, for example, illustrates an indicator 150 that is adapted to move to indicate capture of a native valve leaflet. The indicator 150 is adapted to indicate capture of a native valve leaflet under imaging. The appearance of the indicator 150 under imaging varies to indicate capture of the native valve leaflet.

The indicator 150 comprises an elongate body. A plurality of elongate bodies is utilized in examples. Each elongate body extends from the valve body 15 to at least one of the one or more anchors 17. For example, a first end of the elongate body is coupled to the valve body 15 and a second end of the elongate body is coupled to a respective one of the anchors 17. The elongate body spans a gap positioned between the valve body 15 and the respective one of the anchors 17. Each elongate body is configured as a wire in examples.

Each elongate body is positioned such that the elongate body extends from a coupling point to the valve body 15 to a respective anchor 17 that is circumferentially aligned with the coupling point on the valve body 15. Each elongate body extends radially outward from the valve body 15 to the respective anchor 17.

Each elongate body is configured to be deflected in a distal direction upon capture of a leaflet by a respective anchor. Each elongate body, for example, is bowed upward or in a proximal direction as shown in FIG. 19. Each elongate body is adapted to be deflected downward or in the distal direction to a deflected position upon capture of a respective native valve leaflet. Each elongate body is flexible in examples and adapted to deflect downward or in the distal direction upon capture of a leaflet. A deflection of the elongate body to a deflected position is visible under imaging to allow a user to determine if capture of a leaflet has occurred.

The indicators 150 include exemplary indicators 150a, b marked in FIG. 19 for identification purposes.

A user views the appearance of the indicator 150 to determine if a force has been applied to the indicator 150, to determine if capture of a native valve leaflet has occurred.

For example, referring to FIG. 20, deployment of the prosthetic valve 152 has occurred. An anchor 17a (shown to the right of the page in FIG. 20) has missed capture of the leaflet 82a. The indicator 150a positioned proximate such anchor 17a accordingly remains in an undeflected position or extends upward or proximally from the valve body 15, which is visible via imaging. The imaging can comprise fluoroscopy or ultrasound, or combinations of fluoroscopy and ultrasound, or other forms of imaging. A user accordingly determines that a failed capture or missed capture of the leaflet 82a has occurred by viewing the undeflected position of the indicator 150a.

An anchor 17b (shown to the left of the page in FIG. 20) has made capture of the leaflet 82b. The indicator 150b proximate such anchor 17b accordingly moves to a deflected position, which can be visible via the imaging. A user accordingly determines that capture of the leaflet 82b has occurred by viewing a deflected position of the indicator 150b. The indicator 150b deflects downward or in a distal direction upon capture of the leaflet 82b.

In examples, each indicator 150 includes one or more markers 154 that improve ease of imaging of the indicator 150. The markers can be configured similarly as the markers discussed with respect to FIGS. 15 and 16.

In examples, a user can determine if capture of a leaflet has occurred by viewing a difference in position between the indicators 150a, b. For example, if the indicator 150a shown to the right of the page in FIG. 20 has a lesser deflection than the indicator 150b shown to the left of the page in FIG. 20, then a user can determine that a missed capture has occurred.

The features of FIGS. 19 and 20 may be utilized solely or in combination with any example disclosed herein.

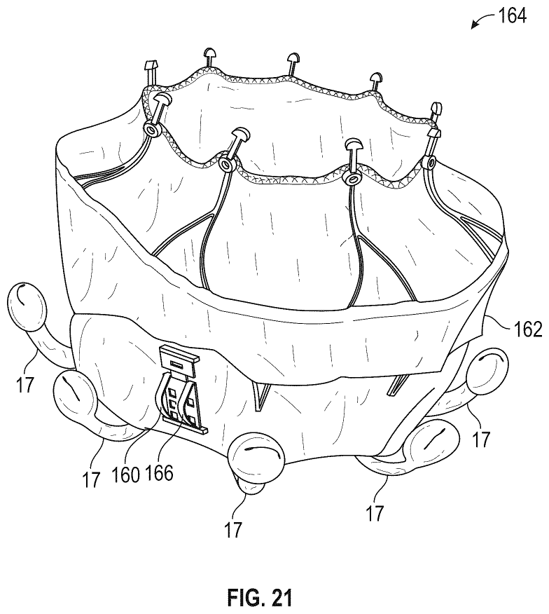

In examples, the configuration of the indicators can be varied as desired. FIG. 21, for example, illustrates an indicator 160 that is adapted to move to indicate capture of a native valve leaflet. The indicator 160 is adapted to indicate capture of a native valve leaflet under imaging. The appearance of the indicator 160 under imaging varies to indicate capture of the native valve leaflet.

The indicator 160 comprises a button that is adapted to be depressed to indicate capture of a native valve leaflet. The indicator 160 is positioned on a valve body 162 of the prosthetic valve 164, which otherwise can be configured similarly as the valve body 15 discussed with respect to FIGS. 1A-2. The indicator 160 protrudes from an outer surface of the valve body 15 in examples.

The indicator 160 is positioned such that contact from a native heart valve leaflet against the indicator 160 presses the indicator 160 inward. The indicator 160 is positioned between adjacent anchors 17 such that a native heart valve leaflet presses against the indicator 160 when the native heart valve leaflet is captured.

The indicator 160 includes a movable protrusion 166 that is adapted to move radially inward as the indicator 160 is depressed. FIG. 22, for example, illustrates a side perspective view of the indicator 160 showing the movable protrusion 166 extending outward from a portion of the frame 168 of the prosthetic valve 164. The movable protrusion 166 comprises one or more arms that can be angled to deflect outward from the frame 168. The one or more arms have a first end portion 170 that couple to the frame 168. The first end portion 170 can couple to the frame 168 via sutures or another form of coupling. The first end portion 170 can comprise a pivot that the movable protrusion 166 can pivot about. A second end portion 172 of the one or more arms includes a marker 174 that improves ease of imaging of the indicator 160. In examples, the marker 174 is adapted to be visible under imaging such as fluoroscopy. The second end portion 172 of the one or more arms and the marker 174 is adapted to rotate about the pivot as the movable protrusion 166 is depressed.

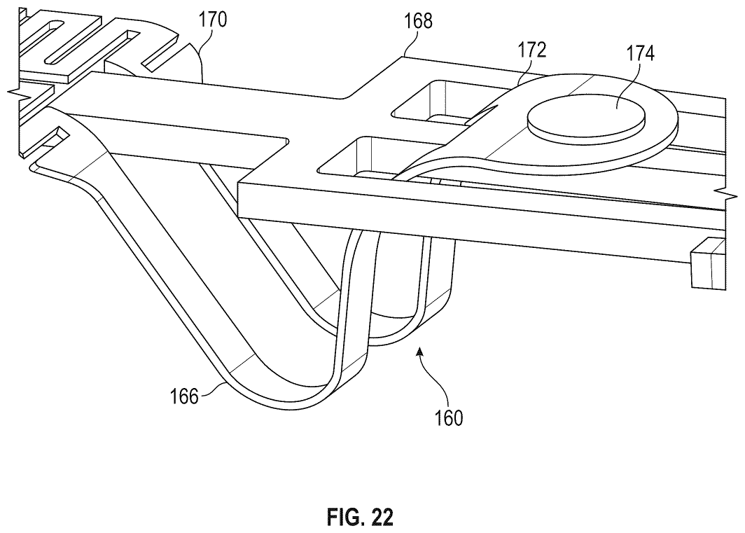

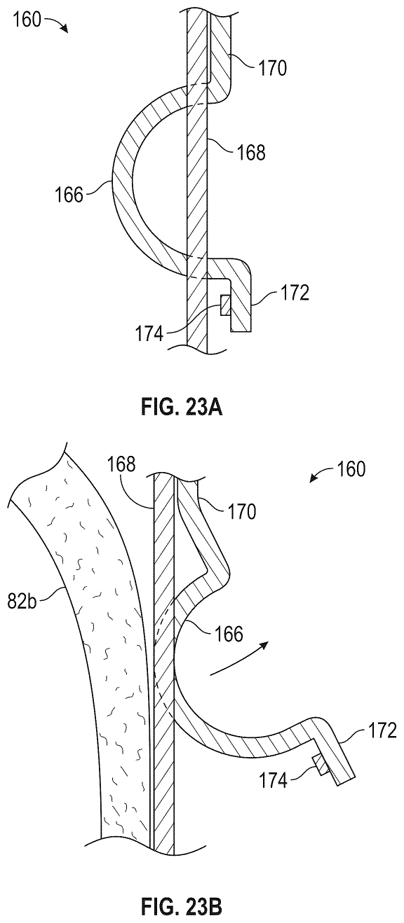

For example, referring to FIG. 23A, a cross-sectional view of the indicator 160 relative to a frame 168 of the prosthetic valve 164 is visible. The extension of the movable protrusion 166 from the frame 168 is shown. Such a configuration represents the lack of a native valve leaflet or other portion of a native heart valve from pressing against the indicator 160.

FIG. 23B illustrates a cross-sectional view of the movable protrusion 166 having been pressed radially inward by a leaflet 82b that has been captured. The leaflet 82b presses against the movable protrusion 166 and move the movable protrusion 166 radially inward. The second end portion 172 of the one or more arms pivots radially inward about the first end portion 170.

A user views the appearance of the indicator 160 to determine if a force has been applied to the indicator 160, to determine if capture of a native valve leaflet has occurred. The imaging can comprise fluoroscopy or ultrasound, or combinations of fluoroscopy and ultrasound, or other forms of imaging. A user accordingly determines that a failed capture or missed capture of a leaflet 82 has occurred by viewing whether the indicator 160 remains in an undeflected position. A user is able to image the deflection of one or more of the arms or of the marker 174.

The features of FIGS. 21-23B may be utilized solely or in combination with any example disclosed herein.

The indicators and/or markers disclosed with respect to FIGS. 1A-23B can comprise a radiopaque material for enhanced visualization. Heavy metals can be utilized, such as gold, platinum, iridium, or tantalum, or other forms of radiopaque materials. In examples, a material such as nitinol or stainless steel may be utilized, which may be visualized under fluoroscopy or ultrasound. Other forms of materials can be utilized as desired.

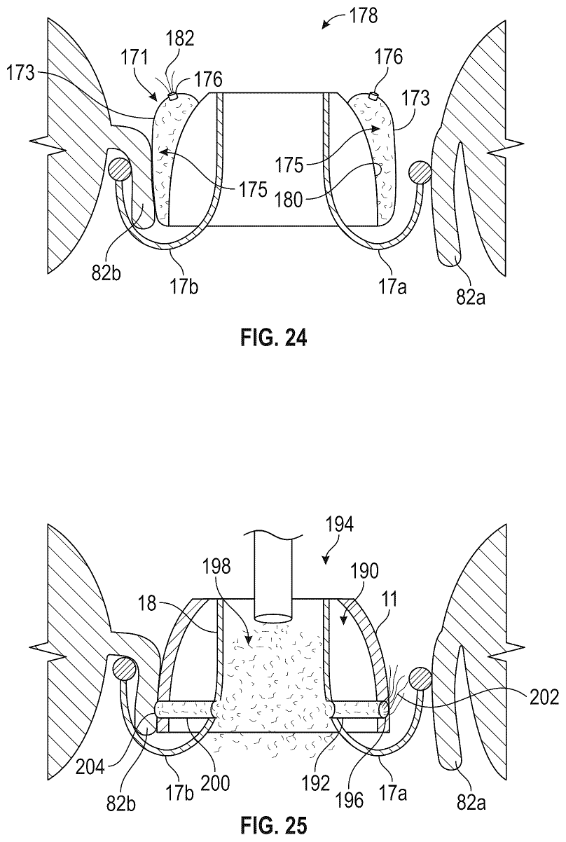

In examples, the configuration of the indicators can be varied as desired. FIG. 24, for example, illustrates an indicator 171 that is adapted to move to indicate capture of a native valve leaflet. The indicator 171 is adapted to indicate capture of a native valve leaflet under imaging. The appearance of the indicator 171 under imaging varies to indicate capture of the native valve leaflet.

The indicator 171 comprises a bladder 173 and contrast material 175 that fills the bladder 173. The bladder 173 is adapted to be filled with the contrast material 175. The bladder 173, in examples, includes one or more openings 176 that are adapted to release the contrast material 175 to indicate capture of a native valve leaflet. The bladder 173, for example, is made of a flexible material that is adapted to deform to cause the contrast material 175 to release from the bladder 173 through the opening 176.

The bladder 173 is positioned such that a native valve leaflet presses against the bladder 173 upon capture of the native valve leaflet. The bladder 173, for example, is positioned between an anchor (exemplary anchors indicated by anchors 17a, b) of the prosthetic valve 178 and a valve body 180 and adapted to be pressed by a native valve leaflet upon capture of the native valve leaflet. In examples, the bladder 173 has other positions such as between adjacent anchors 17 or otherwise in position to receive pressure from the native valve leaflets.

The openings 176 comprise valves that allow for release of contrast material 175 upon a certain amount of pressure being applied to the bladder 173. For example, the openings 176 comprise check valves or other forms of valves for allowing release of the contrast material 175 upon application of pressure.

The openings 176 are positioned at locations circumferentially spaced from each other. Such circumferential spacing allows for a determination if anchoring at the various circumferential locations has occurred. For example, an opening 176 is placed at the circumferential position of each anchor 17, or at another spacing as desired. In examples, multiple bladders are provided at circumferential spacing from each other. Each bladder has an opening for releasing contrast material that indicates capture of a native valve leaflet at the location of that respective bladder.

The bladder 173 is filled with the contrast material 175 upon deployment of the prosthetic valve 178 to an implantation site. The openings 176 retain the contrast material 175 within the bladder 173 in such a configuration. Referring to the right side of FIG. 24, the anchor 17a has failed to capture or missed capture of a leaflet 82a. The bladder 173 accordingly does not have pressured applied to it by the leaflet 82a and does not emit the contrast material 175 at that portion of the bladder 173.

Referring to the left side of FIG. 24, the anchor 17b has captured the leaflet 82b. Accordingly, the bladder 173 releases the contrast material 182 from the opening 176. The release of the contrast material 182 can be imaged for a user to determine that capture of the leaflet 82b has occurred.

A user views the appearance of the indicator 171 to determine if a force has been applied to the indicator 171, to determine if capture of a native valve leaflet has occurred. The imaging comprises fluoroscopy or ultrasound, or combinations of fluoroscopy and ultrasound, or other forms of imaging. A user accordingly determines that a failed capture or missed capture of the leaflet 82a has occurred by viewing whether the indicator 171 fails to release the contrast material 175.

The features of FIG. 24 may be utilized solely or in combination with any example disclosed herein.

In examples, the configuration of the indicators may be varied as desired. FIG. 25, for example, illustrates an indicator 190 that is adapted to indicate capture of a native valve leaflet under imaging.

The indicator 190 comprises a channel 192 adapted to pass contrast material therethrough. The channel 192 extends from an interior of the prosthetic valve 194 to an exterior surface of the prosthetic valve 194 such that contrast material is passed from the interior to the exterior surface of the prosthetic valve 194. The channel 192, for example, passes from interior of the valve frame or inner frame 18 to the outer frame or sealing body 11. An opening 196 of the channel 192, for example, is positioned on a surface of the outer frame or sealing body 11.

The channel 192 is positioned such that a native valve leaflet presses against the opening 196 of the channel 192 to cover the opening 196. The flow of contrast material through the channel 192 accordingly may not be impeded and a user visualizing the flow of contrast material can determine that capture of the leaflet 82a has not occurred. In an example in which the opening 196 of the channel is covered by a leaflet 82a, then flow of material through is impeded and a user can determine that capture of the leaflet 82a has occurred.

In examples, the prosthetic valve 194 includes a plurality of channels and openings that are positioned at locations circumferentially spaced from each other. Such circumferential spacing allows for a determination if anchoring at the various circumferential locations has occurred. Openings of the channels are positioned at locations at which the native valve leaflets will be anchored to.

Upon deployment of the prosthetic valve 194 to an implantation site, a contrast material 198 can be flowed through the interior of the prosthetic valve 194. The contrast material flows into the channel 192 and flows into another channel 200. Referring to the right side of FIG. 25, the anchor 17a has failed to capture or missed capture of a leaflet 82a. The opening 196 accordingly remains uncovered and does not impede flow of contrast material therethrough. As such, contrast material 202 is released that is visible on the outside of the prosthetic valve 194. The presence of such contrast material 202 following deployment of the prosthetic valve 194 indicates that the anchor 17a has failed to capture or missed capture of the leaflet 82a.

Referring to the left side of FIG. 25, the anchor 17b has captured the leaflet 82b. Accordingly, an opening 204 of the channel 200 is covered by the leaflet 82b such that the contrast material is impeded from flowing therethrough. The lack of contrast material visible on the outside of the prosthetic valve 194 at the opening 204 indicates that the anchor 17b has captured the leaflet 82b.

A user views the appearance of the indicator 190 to determine if capture of a native valve leaflet has occurred. The imaging can comprise fluoroscopy or ultrasound, or combinations of fluoroscopy and ultrasound, or other forms of imaging. A user accordingly can determine that a failed capture or missed capture of the leaflet 82a has occurred by viewing whether the contrast material 202 is released. The contrast material utilized in the examples of FIGS. 24 and 25 can comprise a radiopaque material or fluid in examples. Iodine or other forms of radiocontrast agents can be utilized.

The features of FIG. 25 may be utilized solely or in combination with any example disclosed herein.

In examples, one or more sensors may be utilized that are adapted to sense a condition within the patient's body. The condition may comprise a condition of a prosthetic valve, for example, whether the prosthetic valve has been deployed in a desired manner or has certain features. For example, the condition may comprise whether capture of a native valve leaflet has occurred. In examples, the condition may comprise whether the prosthetic valve is operating in a desired manner within the patient's body. In examples, the condition may comprise an environmental condition within the patient's body (e.g., a pressure within the patient's body or a flow through a valve of the patient's body). Various other conditions can be sensed. The one or more sensors can be adapted to be coupled to a prosthetic heart valve that can be deployed to a native valve of a patient's heart.

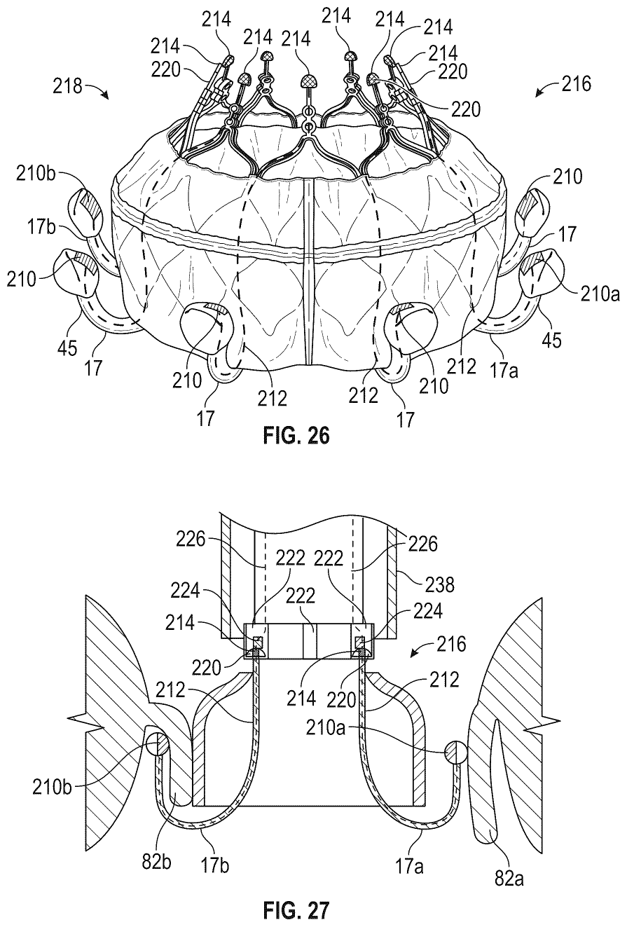

Referring to FIG. 26, in examples, one or more sensors are provided that detect whether capture of a native valve leaflet has occurred. The one or more sensors may comprise an indicator that is adapted to indicate capture of a native valve leaflet by one or more anchors via a signal provided by the one or more sensors. The one or more sensors are adapted to sense whether at least one of the anchors has captured a native valve leaflet.

In examples, the sensors 210 comprise contact sensors that are adapted to sense contact between the sensors 210 and a surface of a native valve (e.g., a surface of a native heart valve leaflet). The sensors 210 can be positioned in a variety of locations, including upon the anchors 17. The sensors 210 can be positioned on the distal portions 45 of the anchors 17, including the distal tips of the anchors 17. The sensors 210 are adapted to be positioned on inward facing surfaces of the anchors 17, such that the sensors 210 can contact a surface of the native valve leaflets upon the anchors 17 anchoring to the native valve leaflets. The sensors 210 can be provided in other positions as desired, such as on a valve body.

The sensors 210 can comprise a variety of forms of sensors. Sensors 210 utilized can comprise one or more piezoelectric sensors, strain gauges, pressure transducers, and/or capacitance sensors. Such forms of sensors can comprise contact sensors and/or force sensors. Other forms of sensors for detecting contact or force may be utilized. In examples, the sensors 210 can comprise an electrode, with a reference electrode positioned on the patient's body. Contact between the electrode and tissue within the patient's body (e.g., the heart valve leaflet) provides an electrical signal between the electrode and the reference electrode that may indicate the contact between the electrode and the heart valve leaflet. Other forms of sensors disclosed herein can be utilized. The sensors may be utilized with any other example disclosed herein.

In examples, a plurality of the sensors 210 are provided, each upon a respective anchor 17. Each sensor 210 is adapted to sense if anchoring to a native valve leaflet has occurred by the respective anchor 17. The sensors 210 include exemplary sensors 210a, b marked in FIG. 26 for identification purposes.

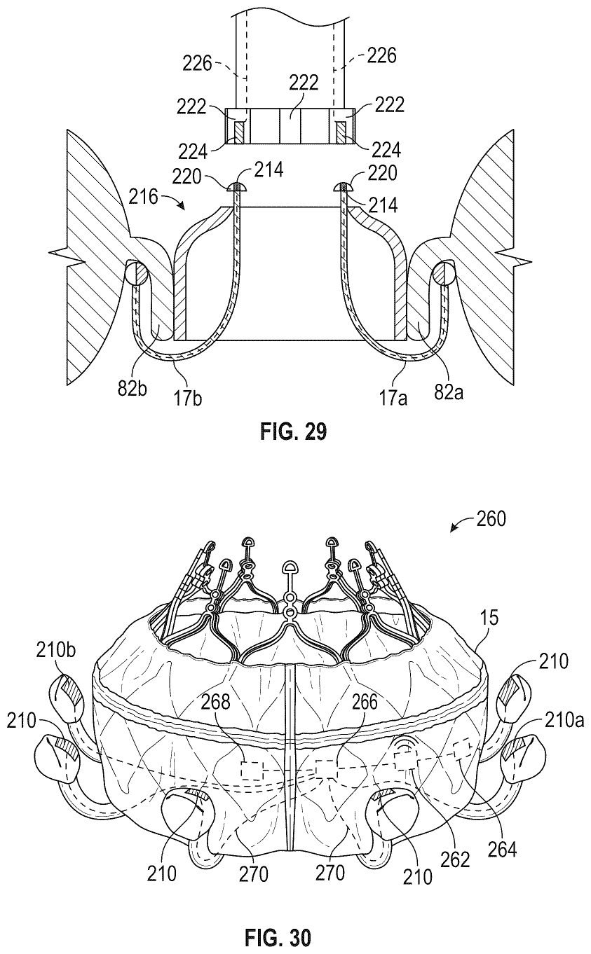

In examples, each of the sensors 210 includes a wired connection to a terminal for the respective sensor. For example, an electrical conduit 212 extends from a sensor 210 to an electrical terminal 214. The electrical conduit 212 extends along the valve body 15 and connect the sensor 210 to the electrical terminal 214. The electrical terminal 214 is positioned in a variety of locations as desired. As shown in FIG. 26, for example, the electrical terminal 214 is positioned at a proximal portion 218 of the prosthetic valve 216. In examples, the electrical terminal 214 is positioned at a coupling portion of the prosthetic valve 216 that is adapted to couple to a delivery apparatus for the prosthetic valve 216. Connectors 220 or end tabs on the prosthetic valve 216 that couple to the delivery apparatus can each include an electrical terminal 214. Such connectors 220 couple to connectors 222 (more marked in FIG. 29) on the delivery apparatus that can couple to the connectors 220 for the prosthetic valve 216.

A delivery apparatus, for example, can include one or more electrical terminals 224 (marked in FIG. 29) that are adapted to contact the electrical terminals 214 of the prosthetic valve 216. The electrical terminals 224 can be coupled to electrical conduits 226 that extend along the delivery apparatus. The electrical conduits 226 convey the signal provided by the sensors 210 to an output for utilization.

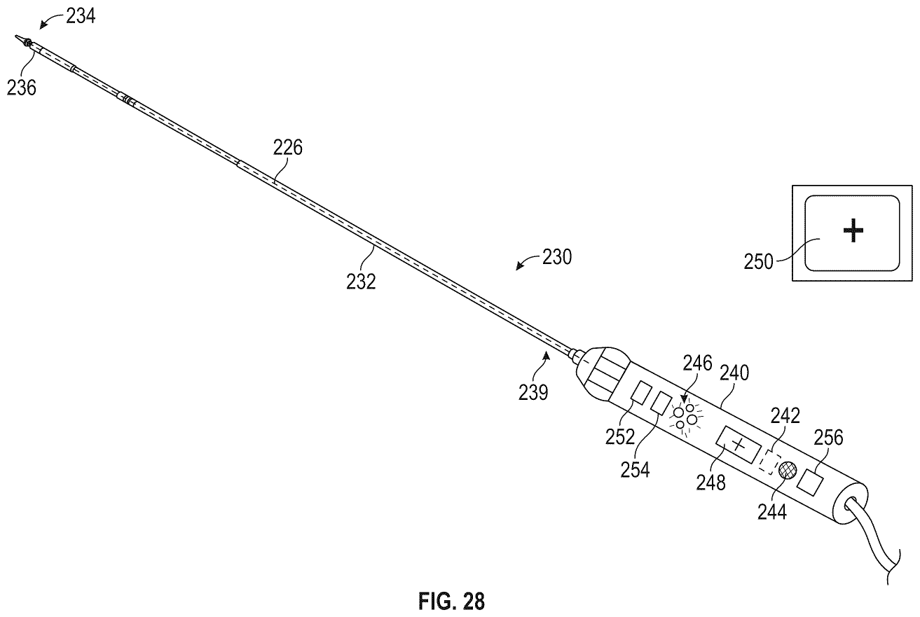

FIG. 28, for example, illustrates an exemplary delivery system 230 that may be utilized in examples herein. The delivery system 230 is configured similarly as the delivery system 70 shown in FIG. 3, yet is adapted to receive signal outputs from the sensors 210. The delivery system 230 includes a delivery apparatus having an elongate shaft 232. The elongate shaft 232 includes a distal portion 234 including an implant retention area 236 (e.g., the capsule 238 shown in FIG. 27 or another form of implant retention area). The implant retention area 236 includes the connectors 222 on the delivery apparatus (marked in FIG. 29). The connectors 222 have a variety of forms and can comprise receiving slots adapted to receive the connectors 220 in the form of tabs, or can have another configuration as desired (e.g., sutures, or another form of connector).

A proximal portion 239 of the elongate shaft 232 is coupled to a housing 240 that can be in the form of a handle for the delivery apparatus. In examples, the housing 240 has other forms.

The electrical conduits 226 extend along the elongate shaft 232 to provide the signal from the sensors 210 to an indicator device adapted to produce an indication of the signal provided by the sensors 210. An indicator device has a variety of forms. For example, referring to FIG. 28, the indicator device comprises a visual indicator, a haptic indicator 242, or an audible indicator 244, among other forms of indicator devices. The audible indicator 244 comprises a speaker or other device adapted to produce an audible indication. The haptic indicator 242 comprises a motor or other device adapted to produce a haptic indication. The visual indicator has a variety of forms. The visual indicator comprises one or more lights 246, or one or more display screens 248, 250, or another device adapted to produce a visual indication. Combinations of indicator devices can be utilized in examples.

In examples, an indicator device is provided on the delivery apparatus or is provided remote from the delivery apparatus. For example, an indicator device is positioned on a housing 240 or handle of a delivery apparatus (as shown with the indicator devices 242, 244, 246, and 248). An indicator device may be positioned remote (such as an indicator device or display screen 250).

The indicator devices can be adapted to provide an indication of the condition sensed by the sensors 210. For example, upon capture of a leaflet, one or more of the indicator devices indicate that capture has occurred. The lights 246 flash or one or more of the display screens 248, 250 can provide a symbol such as a “+” shown in FIG. 28 or another form of indication. In examples, the indicator devices are adapted to provide an indication of a failed capture or missed capture of a leaflet. For example, the lights 246 can flash in a certain manner to indicate a failed capture (e.g., a color of the lights can change, or a light may lack illumination corresponding to the missed point of capture), or a display screen 248, 250 can indicate a failed capture or missed capture. For example, a display screen 248, 250 displays a symbol such as a “−” symbol to indicate a failed capture or missed capture.

In examples, the signal from the sensor 210 is provided directly to the indicator devices. In examples, the signals are received by a processor 252 that processes the signals provided by the sensors 210. The processor 252 can be configured to operate based on programming provided in a memory 254. The processor 252 receive the signals and can determine an output to provide to one or more of the indicator devices based on the signals.

The processor 252 has a variety of forms, and comprises a microprocessor, a controller, or a plurality of processors utilized in combination, among other forms of processors. The memory 254 comprises a hard drive (e.g., mechanical or solid state) or can comprise flash memory, or RAM, or ROM, among other forms of memory. The memory stores data or instructions that is non-transitory and for use by the processor 252. In examples, a distributed processing unit, or remote processing unit can be utilized. For example, a cloud computing environment, or remote processing utilizing the internet or a wireless network can be utilized.

In examples, the processor 252 and memory 254 are positioned on the handle or housing 240. A power source 256 is provided that powers the processor 252 and memory 254 and other components of the delivery system 230 (e.g., the indicator devices 242, 244, 246, and 248). The power source 256 comprises a battery or other form of power source (e.g., a wired or wireless connection to an external power source) for powering the components of the delivery system 230. In examples, the processor 252, memory 254 or other components of the delivery system 230 can be provided remote from the handle or housing 240.

The elongate shaft 232 is adapted to be deflected via operation of a control mechanism, in a similar manner as the elongate shaft shown in FIG. 3. The elongate shaft 232 can be navigated to a desired implantation site.

FIG. 27 illustrates an exemplary implementation of the sensors 210 to sense a condition within a patient's body, for example whether the anchors 17a, b have captured a respective native valve leaflet 82a, b. As shown, the prosthetic valve 216 is deployed with the electrical terminals 214 of the prosthetic valve 216 remaining in electrical connection with the terminals 224 of the delivery system 230. The connectors 220 of the prosthetic valve 216, for example, remain coupled to the connectors 222 of the delivery system 230. In such a configuration, a signal from the sensors 210 is provided through the electrical conduits 212 and is transmitted to the electrical terminals 224 and electrical conduits 226 to provide an output of the sensor 210 signals.

Referring to the right side of FIG. 27, an anchor 17a has failed to capture or missed capture of the leaflet 82a. As such, the sensor 210a fails to contact the leaflet 82a. Accordingly, the sensor 210a indicates a lack of capture of the leaflet 82a. The sensor 210a provides a signal indicating a lack of capture or provides a lack of a signal corresponding to a lack of capture of the leaflet 82a.

Referring to the left side of FIG. 27, an anchor 17b has captured the leaflet 82b. As such, the sensor 210b contacts the leaflet 82b. Accordingly, the sensor 210b indicates capture of the leaflet 82b. The sensor 210b provides a signal indicating capture of the leaflet 82b.

The signals are transmitted to one or more of the indicator devices 242, 244, 246, 248, and 250. The indicator devices indicates whether capture has occurred. The indicator devices indicate the missed capture of the leaflet 82a or the capture of the leaflet 82b, or a combination of the missed capture and the capture. In an example in which the anchors 17a, b are each properly deployed and capture respective leaflets, then the indicator devices indicate the respective captures of the leaflets. In an example in which the anchors 17a, b each fail to capture a leaflet 82a, b, then the indicator devices indicate a lack of capture of a leaflet 82a, b.

The prosthetic valve 216 can be redeployed to allow the anchor 17a to anchor to the heart valve leaflet 82a. FIG. 29, for example, illustrates such a configuration.

In examples, the delivery system can be withdrawn from the prosthetic valve 216 following deployment. FIG. 29, for example, illustrates the delivery system 230 having been withdrawn. The connectors 220 of the prosthetic valve 216 disengage from the connectors 222 of the delivery system 230. Similarly, the electrical terminals 214 of the prosthetic valve 216 disengage from the electrical terminals 224. The prosthetic valve 216 accordingly remains implanted within the patient's body with the delivery system 230 withdrawn.

In examples, other forms of transmission of signals from the sensors 210 can be utilized. In examples, a wireless transmission from sensors 210 can be utilized.

Referring to FIG. 30, in examples, a prosthetic valve 260 is adapted to wirelessly transmit signals from the sensors 210. The signals can be of a condition sensed within the patient's body.

A wireless transmitter 262 is utilized to transmit the signals from the prosthetic valve 260. The wireless transmitter 262 can be coupled to the prosthetic valve 260 and is positioned, for example, on the valve body 15. The wireless transmitter 262 is positioned on other locations as desired.

In examples, a power source 264 is provided for the wireless transmitter 262. The power source 264 comprises a battery or other form of power source that provides energy for the wireless transmitter 262. In examples, the power source 264 comprises a rechargeable power source that can be recharged via wireless charging (e.g., induction) or via kinetic movement of the patient. The power source 264 provides power to other components of the prosthetic valve 260 in examples (e.g., the sensors).

In examples, a processor 266 is utilized that receives the signals from the sensors 210. The processor 266 processes the signals based on programming stored in a memory 268 to provide data to the wireless transmitter 262 for transmission. The sensors 210 provide electrical signals to the processor 266 via electrical conduits 270. In examples, the use of a separate processor 266 is excluded and the signals are provided directly to the wireless transmitter 262 for transmission.

Referring to FIG. 31, an exemplary transmission by the wireless transmitter 262 is shown. To the right side of FIG. 31, the anchor 17a has failed to capture or has missed capture of the leaflet 82a. As such, the sensor 210a fails to contact the leaflet 82a. Accordingly, the sensor 210a indicates a lack of capture of the leaflet 82a. The sensor 210a provides a signal indicating a lack of capture or provides a lack of a signal corresponding to a lack of capture of the leaflet 82a. A signal can be transmitted wirelessly from the wireless transmitter 262 to a receiver 269.

The receiver 269 is positioned exterior of the patient's body, or in examples, is positioned interior of the patient's body. The receiver 269 comprises a wireless receiver adapted to receive the wireless signals from the wireless transmitter 262. The receiver 269 is adapted to process the signals from the wireless transmitter 262 and cause one or more indicator devices (such as indicator device 250 or another form of indicator device) to provide an indication of the missed capture of the native leaflet by the anchor 17a.

Referring to the left side of FIG. 31, the anchor 17b has captured the leaflet 82b. As such, the sensor 210b contacts the leaflet 82b. The sensor 210b provides a signal indicating capture of the leaflet 82b. A signal is transmitted wirelessly from the wireless transmitter 262 to the receiver 269 indicating the capture.

The receiver 269 causes one or more indicator devices (such as indicator device 250 or another form of indicator device) to provide an indication of the capture of the native leaflet by the anchor 17b.

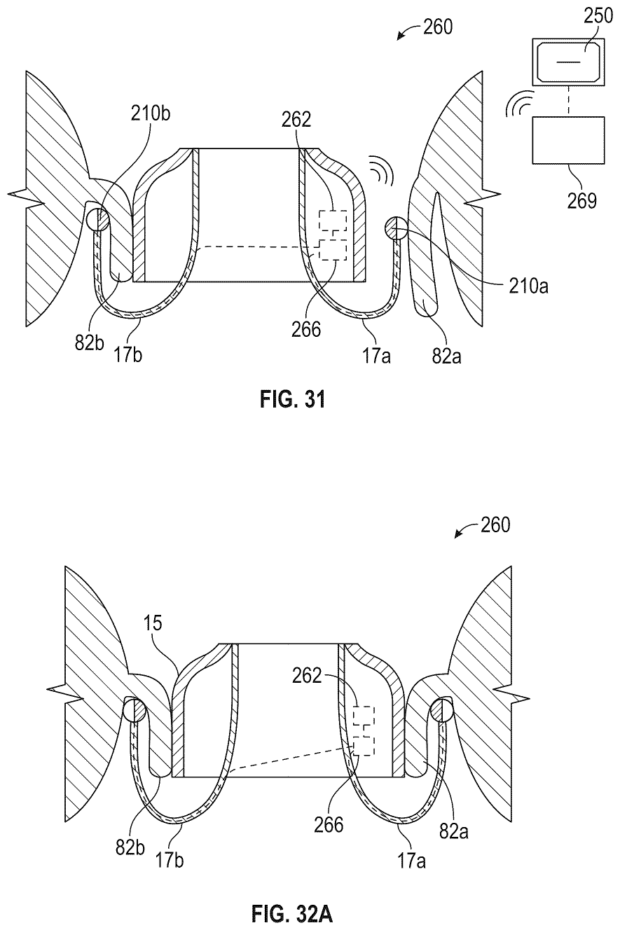

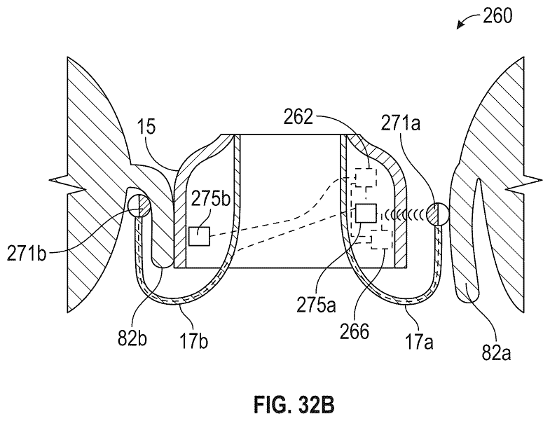

The prosthetic valve 260 can be redeployed to allow the anchor 17a to anchor to the heart valve leaflet 82a. FIG. 32A, for example, illustrates such a configuration. The wireless transmitter 262 provides a wireless signal indicating that capture of the leaflets 82a, 82b has occurred.

In examples, each sensor 210 can comprise a wireless sensor. Each sensor 210 can include a wireless transmitter integrated as part of the sensor 210 and adapted to transmit a wireless signal to a receiver in a similar manner as the wireless transmitter 262.

In examples, other forms of sensors are utilized to determine if capture of a heart valve leaflet has occurred. For example, one or more of proximity sensors, force sensors, optical sensors, or chemical sensors can be utilized to determine if capture has occurred.

A proximity sensor, for example, comprises a sensor that utilizes a detection in a variation of an electromagnetic wave or magnetic field to sense proximity of a leaflet. Such a sensor can comprise an electrical impedance sensor. Proximity sensors such as infrared, electromagnetic, capacitive, or ultrasound proximity sensors can be utilized.

A force sensor senses a force of contact upon the force sensor.

An optical sensor can utilize light such as infrared or other forms of light that can be blocked by a native valve leaflet to determine capture of a leaflet. Optical sensors can include emitters and receivers in examples. For example, referring to FIG. 32B, an emitter 271a can be positioned on an anchor 17a of a prosthetic valve 273. The emitter 271a can comprise an optical emitter adapted to emit light (such as infrared or other forms of light) for being received by an optical receiver 275a. The optical receiver 275a, for example, can be positioned on the valve body 15 or on another location as desired.

Referring to FIG. 32B, the optical receiver 275a receives the light from the emitter 271a, which indicates a failed capture or missed capture of the leaflet 82a by the anchor 17a. Accordingly, the optical receiver 275a provides a signal for transmission by the wireless transmitter 262 indicating the failed capture or missed capture of the leaflet 82a.

An emitter 271b can be provided that can be configured similarly as the emitter 271a. The emitter 271b can emit light for being received by the optical receiver 275b. As such, upon capture of the leaflet 82b, the light from the emitter 271b can be blocked by the leaflet 82b and not received by the optical receiver 275b. The optical receiver 275b provides a signal for transmission by the wireless transmitter 262 indicating the capture of the leaflet 82b.

In examples, the positions of the emitters 271a, b and the receivers 275a, b can be reversed as desired. Other positions of emitters and receivers can be utilized in examples. Other forms of optical sensors can be utilized.

A chemical sensor may comprise a biosensor that senses contact with tissue of a native valve leaflet.

In examples, the position of the sensors can be varied as desired. For example, one or more sensors can be positioned on the valve body 15 or another portion of a prosthetic valve to sense whether capture of a leaflet has occurred. One or more sensors can be positioned on a combination of an anchor and the valve body as desired.

The features of FIGS. 26-32B may be utilized solely or in combination with any example disclosed herein.

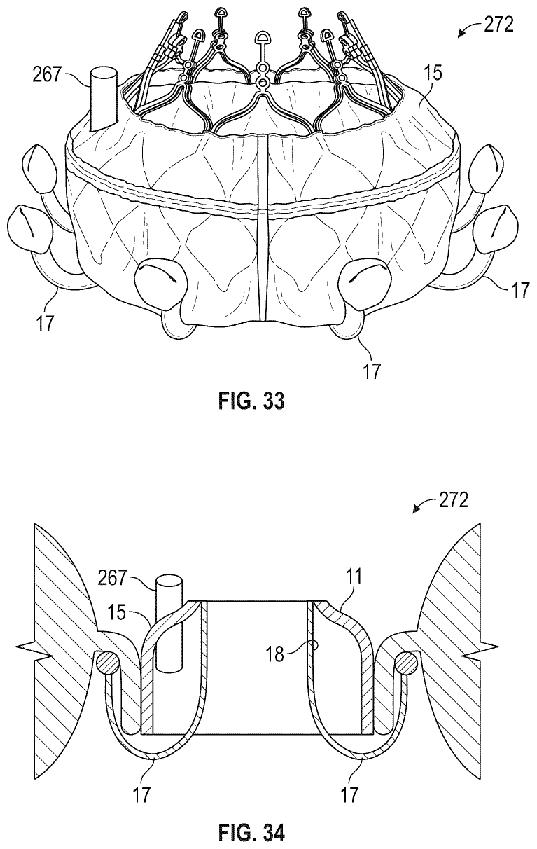

In examples, other conditions within a patient's body can be sensed by one or more sensors. For example, referring to FIG. 33, a pressure within a patient's body is sensed. The pressure comprises a pressure within at least one chamber of a heart. Pressure can be sensed for a variety of reasons, including determination of proper operation of a prosthetic valve, determination of a health condition of the patient's heart, or a determination that proper remodeling is occurring after treatment (e.g. a remodeling of a ventricle such as a right ventricle for tricuspid valve deployment). The pressure can be utilized to determine if a patient is recovering from a procedure, or may need another procedure.

A sensor in the form of a pressure sensor 267 can be utilized to sense a pressure in one or more chambers of a heart. The pressure sensor 267 is positioned upon a prosthetic valve 272. The pressure sensor 267 is positioned to determine a pressure within a proximal chamber or atrial chamber of the heart and can be exposed to fluid pressure on the proximal or atrial side of the heart.

Referring to FIG. 34, in examples, the pressure sensor 267 can be positioned on the valve body 15. In examples, other locations can be utilized (e.g., on the anchors 17 or at another location as desired). The pressure sensor 267 can be positioned between an outer frame or sealing body 11 and an inner body or inner frame 18 of the prosthetic valve 272. The pressure sensor 267 protrudes from the surface of the valve body 15 and is positioned within the proximal or atrial side of the heart.

In examples, the pressure sensor 267 is positioned to sense a pressure within a distal chamber or ventricular chamber of a heart. The location of the pressure sensor 267 is varied to allow for pressure sensing within the distal chamber or ventricular chamber. The pressure sensor 267 is exposed to fluid within the distal chamber or ventricular chamber.

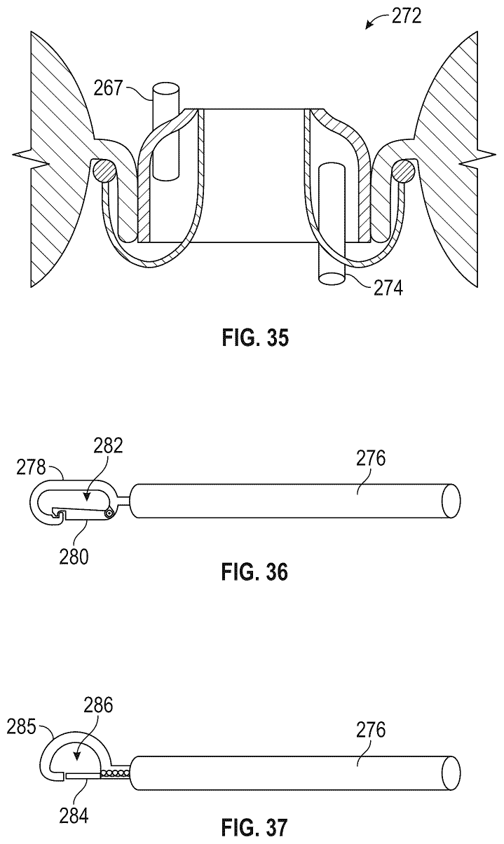

In examples, one or more pressure sensors can be adapted to sense a pressure in both a proximal or atrial chamber and a distal or ventricular chamber. For example, a pressure sensor is adapted to sense both pressures and provide a signal indicating both pressures. A pressure differential can be determined in examples. Referring to FIG. 35, in examples, a first pressure sensor 267 is adapted to sense a pressure within a proximal or atrial chamber and a second pressure sensor 274 is adapted to sense a pressure within a distal or ventricular chamber. In examples, a greater number of pressure sensors can be utilized as desired.

The one or more pressure sensors are utilized to transmit wireless signals indicating a pressure that is sensed. For example, a configuration described with respect to FIGS. 30 and 31 is utilized to allow for wireless transmission of signals from the one or more pressure sensors. In examples, a wired connection between the pressure sensors is utilized to transmit signals from the one or more pressure sensors.

The one or more pressure sensors are configured to transmit the signal of the sensed pressure to a receiver. The one or more pressure sensors may transmit the signal for a user to determine a condition within a patient's body. For example, the user can determine if proper implantation of the prosthetic valve 272 has occurred or if other health conditions of the patient are at issue (e.g., a pressure within the patient's body that is undesired). The signals can be sent at the time of implantation of the prosthetic valve 272 or following implantation. For example, the signals are sent following an implantation procedure to allow for monitoring of the patient in a recovery period or generally following the use and implantation of the prosthetic valve 272. In examples, the signals are provided to one or more of the indicator devices 242, 244, 246, 248, 250 discussed with respect to FIG. 28, or another form of indicator device.

In examples, the one or more pressure sensors are integral with the prosthetic valve 272. In examples, one or more of the pressure sensors can be attached onto the prosthetic valve 272.

FIG. 36, for example, illustrates a pressure sensor 276 that is attached to a prosthetic valve. A coupler 278 is attached to the pressure sensor 276 and is adapted to attach the pressure sensor 276 to a prosthetic valve. The coupler 278 comprises a clip that is adapted to clip onto a portion of a prosthetic valve. The sensor accordingly is adapted to be clipped to the prosthetic valve. The clip includes a pivotable arm 280 adapted to pivot open to allow a portion of the prosthetic valve to fit into a recess 282. Other forms of couplers can be utilized in examples. For example, FIG. 37 illustrates a coupler 285 in the form of a clip having a slidable arm 284 adapted to slide open to allow a portion of the prosthetic valve to fit into a recess 286. The respective arms 280, 284 can be biased (e.g., spring biased) towards a closed state to close the respective recess 282, 286 and retain a portion of the prosthetic valve therein.

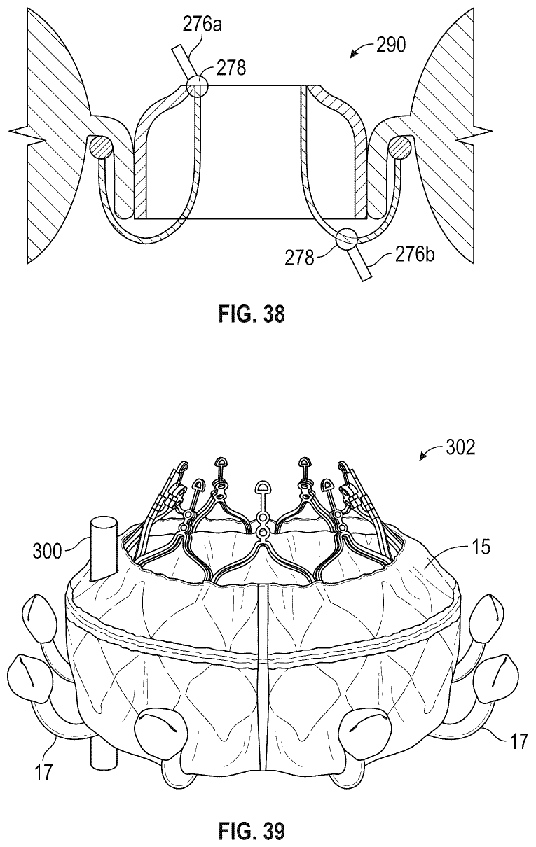

Referring to FIG. 38, a prosthetic valve 290 can have one or more of the pressure sensors 276 attached onto the prosthetic valve 290. A first pressure sensor 276a, for example, is coupled to a proximal portion of the prosthetic valve 290. A second pressure sensor 276b, which is configured similarly as the first pressure sensor 276a, can be coupled to a distal portion of the prosthetic valve 290. The respective pressure sensors 276a, b are adapted to measure the respective pressures on a proximal or atrial side of the prosthetic valve 290 and a distal or ventricular side of the prosthetic valve 290.

The pressure sensors 276a, b can be adapted to be attached to the prosthetic valve 290 by being clipped to a frame of the prosthetic valve 290. The coupler 278 can be attached to struts of a frame of the prosthetic valve 290. The pressure sensors 276a, b can be placed in a variety of locations as desired. The pressure sensors 276a, b, can be provided via a delivery apparatus in a procedure following the implantation of the prosthetic valve 290, or prior to, or during an implantation procedure as desired.

The pressure sensors 276a, b may be adapted to provide wireless signals (for example as described with respect to FIGS. 30 and 31), or may provide wired signals in examples.

The features of FIGS. 33-38 may be utilized solely or in combination with any example disclosed herein.

In examples, other conditions within a patient's body may be sensed by one or more sensors. For example, referring to FIG. 39, a temperature within a patient's body can be sensed. The temperature can comprise a temperature within at least one chamber of a heart.

A temperature sensor 300 can be coupled to a prosthetic valve 302. The temperature sensor 300 can be adapted to sense a temperature on a proximal or atrial side of a prosthetic valve 302. The temperature sensor 300 can be adapted to provide a signal indicating the sensed temperature, in a similar manner as the pressure sensors discussed with respect to FIGS. 33-38.

In examples, the temperature sensor 300 or another temperature sensor is utilized to sense a temperature on the distal or ventricular portion of the prosthetic valve. For example, the temperature sensor 300 has a portion configured to sense the temperature on the distal or ventricular portion of the prosthetic valve. The temperature sensor 300 is adapted to provide a signal indicating the sensed temperature, in a similar manner as the pressure sensors discussed with respect to FIGS. 33-38.

In examples, the temperature sensor 300 is adapted to sense a temperature on a proximal or atrial side of the prosthetic valve and on a distal or ventricular side of the prosthetic valve. The temperature sensor 300 is adapted to provide a signal indicating the temperature difference on the proximal and distal side of the prosthetic valve.

Signals from one or more temperature sensors 300 are utilized to determine flow if desired. For example, a Swan-Ganz catheter method can be utilized to determine the valve's function and blood flow based on the sensed temperature.

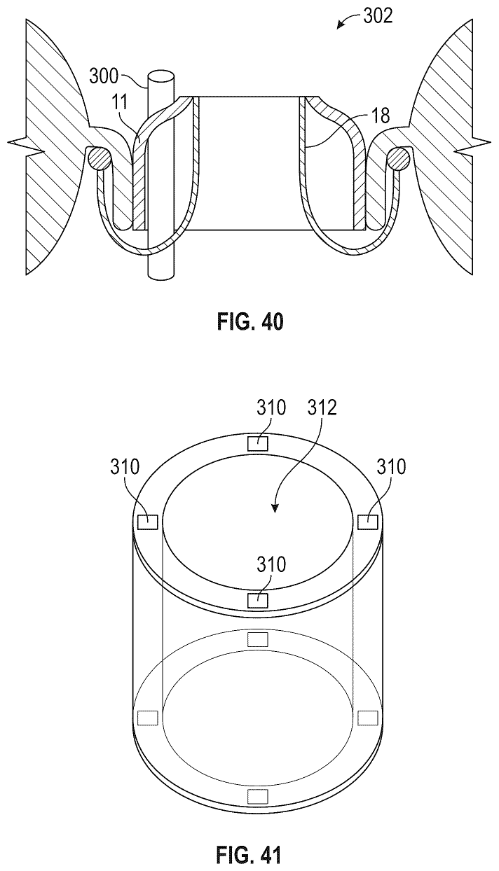

The temperature sensor 300 can be positioned as desired. For example, referring to FIG. 40, the temperature sensor 300 is positioned between the valve frame or inner frame 18 of the prosthetic valve and the outer frame or sealing body 11 of the prosthetic valve 302.

In examples, other conditions within a patient's body may be sensed by one or more sensors. For example, referring to FIG. 41, a flow within a patient's body may be sensed. The flow may comprise a fluid flow within at least one chamber of a heart. The fluid flow may be through a prosthetic valve for example.

One or more flow sensors 310 are arranged to sense flow through a flow channel 312. The flow sensors 310 are positioned circumferentially about the flow channel 312 and adapted to sense a flow through the channel 312. The flow sensors 310, for example, determine a rate of flow through the channel 312. The flow channel 312 comprises a flow channel that one or more prosthetic valve leaflets control flow through, similar to the flow channel 27 marked in FIG. 2.

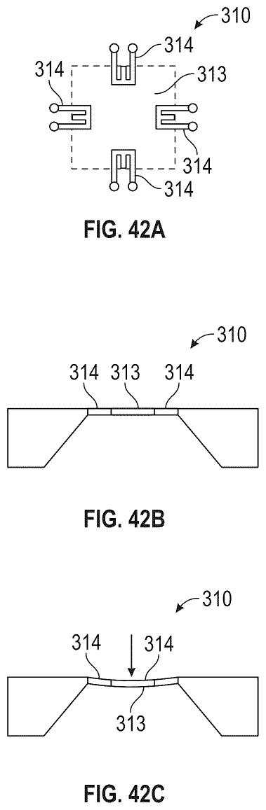

The flow sensors 310 have a variety of forms. The flow sensors 310 comprise piezoelectric sensors in examples. For example, referring to FIG. 42A, a flow sensor 310 includes a deflection surface 313 and a plurality of piezoelectric transducers 314. The piezoelectric transducers 314 are adapted to sense deflection of the deflection surface 313 and provide an electrical signal indicating the deflection of the deflection surface 313. The deflection surface 313 deflects in response to a pressure or flow produced through the flow channel 312. The amount of deflection varies the resistance or voltage produced by the piezoelectric transducers 314, among other features.

FIG. 42B, for example, illustrates a deflection surface 313 that is undeflected. FIG. 42C illustrates the deflection surface 313 having been deflected. The transducers 314 sense the movement of the deflection surface 313 and provide an electrical signal corresponding to the amount of deflection of the deflection surface 313.

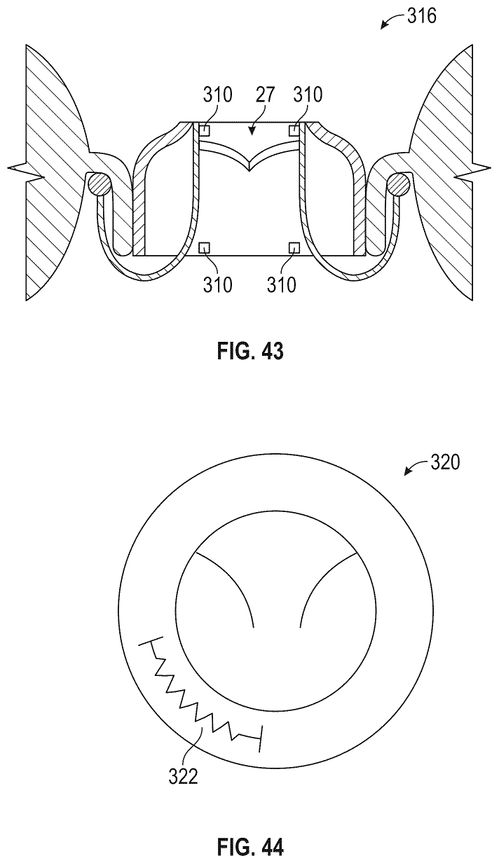

Referring to FIG. 43, the flow sensors 310 are positioned about a flow channel that comprises the flow channel 27 of the prosthetic valve 316. The flow sensors 310 are adapted to sense flow through the prosthetic valve 316 to determine the flow condition through the prosthetic valve 316. The flow sensors 310 can be utilized to indicate potential valve thickening or malperformance as desired.

The flow sensors 310 are adapted to provide a signal indicating the sensed flow, in a similar manner as the pressure sensors discussed with respect to FIG. 33-38. For example, wireless or wired signals may be provided.

The features of FIGS. 41-43 may be utilized solely or in combination with any example disclosed herein.

In examples, other conditions within a patient's body can be sensed by one or more sensors. For example, referring to FIG. 44, a force applied to at least a portion of the heart by a portion of a prosthetic valve 320 can be sensed via a force sensor 322.

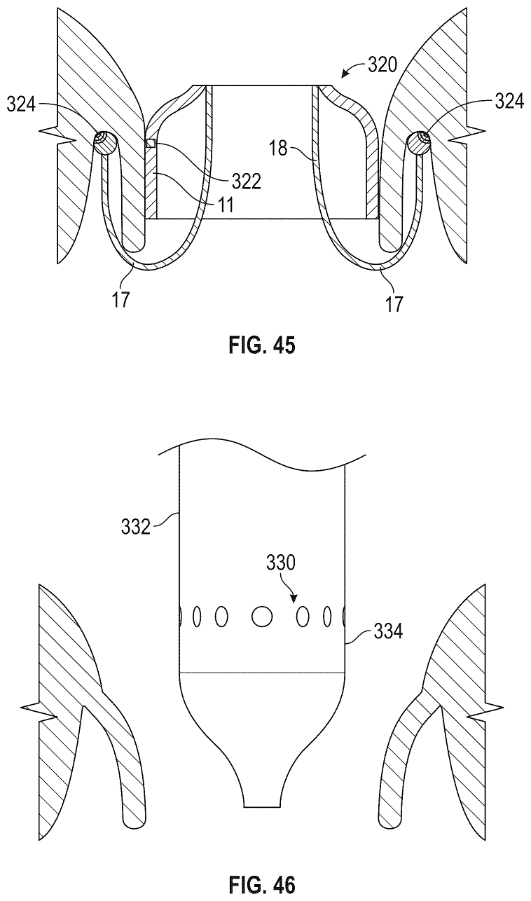

The force sensor 322 as shown in FIG. 44 is adapted to sense a force applied by the prosthetic valve 320 to the native heart valve. The force is applied by an outer frame or sealing body 11 of the prosthetic valve 320. For example, referring to FIG. 45, the force sensor 322 is positioned to sense a force applied by the sealing body 11 against the annulus of the native heart valve. As such, a measure of strain upon the annulus can be determined. In examples, the force sensor 322 comprises a strain gauge. A sensing of annular remodeling and overload detection can be provided as desired.

Further, a force sensor 324 is positioned upon an anchor 17 in examples. The force sensor 324 detects if an anchor 17 is providing an undue force against a native heart valve, which can result in an electrical conduction disturbance or other undesired result for the native heart valve.

The force sensors are adapted to provide a signal indicating the sensed flow, in a similar manner as the pressure sensors discussed with respect to FIG. 33-38. For example, wireless or wired signals can be provided.

The features of FIGS. 44 and 45 may be utilized solely or in combination with any example disclosed herein.

In examples, combinations of sensors may be utilized as desired.

Features of the prosthetic valves shown in FIGS. 26-45 can include features of other prosthetic valves disclosed herein (e.g., a prosthetic valve as disclosed with respect to FIG. 1A-3, or other forms of prosthetic valves). Other forms of prosthetic valves are utilized in examples as desired.

In examples, a delivery apparatus has one or more sensors 330 that are adapted to sense a spatial relationship between the delivery apparatus and at least a portion of a native heart valve.

For example, referring to FIG. 46, a delivery apparatus is configured similarly as a delivery apparatus discussed with respect to FIG. 3 or discussed with respect to FIG. 28. The delivery system comprising the delivery apparatus, for example, includes an elongate shaft. The elongate shaft includes a distal portion 332 including an implant retention area in the form of a capsule 334. The capsule 334 is adapted to retract for release of the implant. A proximal portion of the elongate shaft 232 is coupled to a housing, which can be configured similarly as housings disclosed herein. The delivery apparatus is adapted to deliver an implant to a native heart valve.

The sensors 330 are positioned on the elongate shaft. For example, the sensors 330 are positioned on the capsule 334 and are adapted to sense a spatial relationship between the capsule 334 and a portion of the native heart valve. The sensors 330 can comprise proximity sensors or comprise contact sensors that are adapted to sense the spatial relationship. The contact sensors are considered to sense contact between the delivery apparatus and a portion of the native heart valve, which may include one or more native leaflets of the native heart valve. The sensors 330 can comprise a variety of forms of sensors. Sensors 330 utilized can comprise one or more piezoelectric sensors, strain gauges, pressure transducers, and/or capacitance sensors. Other forms of sensors for detecting contact or force may be utilized. In examples, the sensors 330 can comprise an electrode, with a reference electrode positioned on the patient's body. Contact between the electrode and tissue within the patient's body (e.g., the heart valve leaflet) provides an electrical signal between the electrode and the reference electrode that may indicate contact between the electrode and the heart valve leaflet. Such forms of sensors can comprise contact sensors and/or force sensors. Proximity sensors such as infrared, electromagnetic, capacitive, or ultrasound proximity sensors can be utilized. Other forms of sensors disclosed herein can be utilized. The sensors may be utilized with any other example disclosed herein.

Referring to FIG. 46, in examples, the sensors 330 are spaced from each other circumferentially about the capsule 334. The spacing of the sensors 330 allows the sensors 330 to determine if one or more of the native valve leaflets is proximate one or more of the sensors 330. For example, if one of the leaflets contacts or closely approaches the sensors 330 then a user can determine that the sensors are proximate the native heart valve and in a position for deployment of an implant.

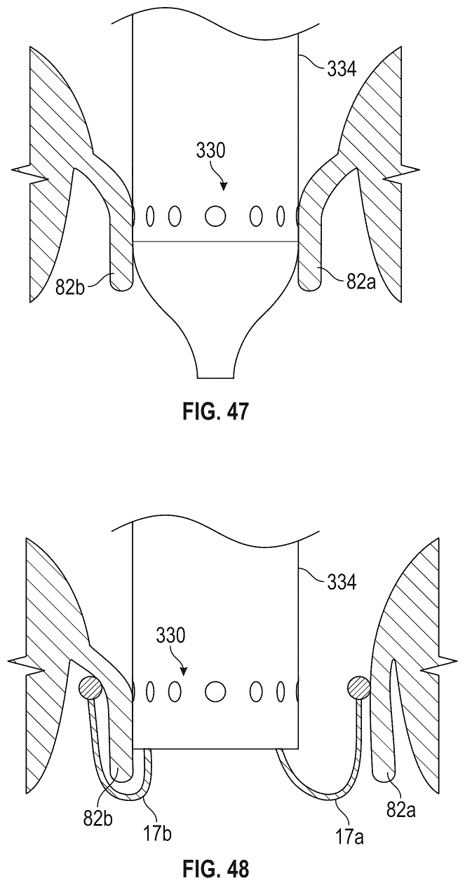

FIG. 47, for example, illustrates the capsule 334 approaching an implantation site and with the native valve leaflets 82a, b in contact with the sensors 330. A signal from the sensors 330 accordingly indicates that the capsule 334 is in position for deployment of an implant contained therein. The axial position of the elongate shaft and capsule 334 is determined by the signal from one or more of the sensors 330.

The sensors 330 can further be utilized to determine if capture of a leaflet has occurred. For example, referring to FIG. 48, anchors 17a, b of a prosthetic valve are deployed from the capsule 334. The anchor 17a may fail to capture or miss capture of the leaflet 82a. As such, one or more sensors 330 produce a signal indicating a lack of contact or proximity with the leaflet 82a. A user accordingly determines that a lack of capture of the leaflet 82a has occurred. One or more sensors 330 indicate that capture of the leaflet 82b has occurred. The one or more sensors 330 for example provide a signal that contact or close proximity with the leaflets 82b has occurred. A user determines that redeployment of the prosthetic valve is to occur for capture of the leaflet 82a.

A first one of the sensors 330 provides a signal indicating contact with a portion of the heart valve, and a second one of the sensors 330 indicates a lack of contact with the portion of the heart valve simultaneously with the first one of the sensors 330 providing the signal.

One or more sensors 330 determine that a capture of a leaflet 82b has occurred, and one or more sensors 330 accordingly determine that a lack of a capture of a leaflet 82a has occurred. Different groups of the sensors 330 produce different signals based on whether a respective leaflet 82a, b has been captured. For example, a first group of sensors 330 proximate the leaflet 82b produce a signal that capture has occurred. A second group of sensors 330 proximate the leaflet 82a produce a signal that capture has not occurred. Different sensors at different locations produce different signals.

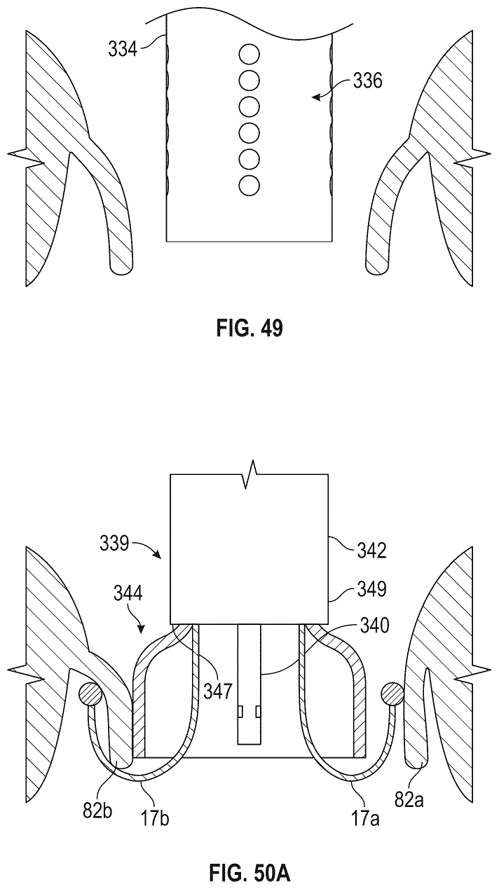

In examples, the configuration of the one or more sensors 330 is varied. For example, referring to FIG. 49, the one or more sensors 336 are positioned in axial alignment upon the capsule 334 of the delivery apparatus. The one or more sensors 336 comprise a plurality of the sensors 336 spaced from each other along the length of the capsule 334. The one or more sensors 336 are aligned with each other along the length of the capsule 334. The one or more sensors 336 are adapted to determine an axial position of the capsule 334 by the signals produced by the one or more sensors 336. For example, a greater depth of the capsule 334 causes different sensors 336 spaced axially along the capsule 334 to provide a signal (e.g., sensors 336 positioned more proximal producing a signal can indicate a greater depth of the capsule 334 relative to the native valve).

The output from any of the sensors can be provided to a user in a variety of manners. Wired signals or wireless signals can be utilized. In examples, an output as represented in FIG. 28 can be utilized. For example, signals can be provided that produce indications on one or more indicator devices 242, 244, 246, 248, 250. For example, different lights 246 can produce different indications based on a depth of the capsule 334 or based on a location of capture or missed capture of a leaflet. One or more of the indicator devices 242, 244, 246, 248, 250 produce an indication of the spatial relationship between the delivery apparatus and at least a portion of the native heart valve sensed by the one or more sensors. One or more of the indicator devices 242, 244, 246, 248, 250 produce an indication of the depth of the delivery apparatus relative to the native heart valve. Other forms of output are provided to a user as desired.

In examples, a processor is utilized, similar to the processor 252 represented in FIG. 28. A memory, similar to the memory 254, can further be utilized. The processor is adapted to receive one or more signal from the sensors. The processor is adapted to determine a capture or missed capture of a native leaflet based on the one or more signals. The processor is adapted to determine a depth of the delivery apparatus relative to a native heart valve based on the one or more signals. Other determinations by a processor may be provided based on the one or more signals.

The features of FIGS. 46-49 may be utilized solely or in combination with any example disclosed herein.

In examples, other forms of indicators may be utilized with a delivery apparatus. For example, any other forms of indicators disclosed herein can be utilized with a delivery apparatus. The indicators can be positioned on a capsule or other portion of a delivery apparatus. The indicators can comprise protrusions that extend radially outward from the delivery apparatus for example. The indicators extend radially outward from a capsule of the delivery apparatus. Indicators 130 shown in FIGS. 15 and 16, or indicators 140 shown in FIGS. 17 and 18, or other forms of indicators, can be utilized as desired. The movement or variation in appearance of the indicators can be utilized to determine proximity to an implantation site or capture or missed capture of native valve leaflets. In examples, other forms of indicators are utilized as desired.

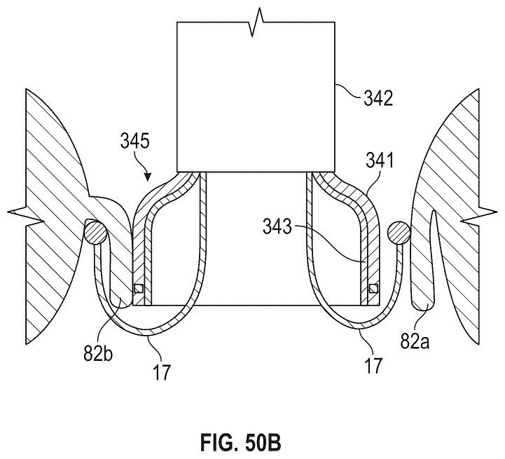

In examples, an imaging apparatus is coupled to a delivery apparatus that can be adapted to image an area external to the delivery apparatus. For example, referring to FIG. 50A, an imaging apparatus 340 is adapted to extend along a delivery apparatus 342. The imaging apparatus 340 can have a variety of forms and may comprise an ultrasound imaging apparatus or a fluoroscopic imaging apparatus, or combinations of such imaging apparatuses. In examples, other forms of imaging apparatuses can be utilized.

The delivery apparatus 342 can be configured similarly as a delivery apparatus discussed with respect to FIG. 3 or discussed with respect to FIG. 28. The delivery system comprising the delivery apparatus, for example, includes an elongate shaft. The elongate shaft includes a distal portion 339 including an implant retention area in the form of a capsule 349. A proximal portion of the elongate shaft can be coupled to a housing, which may be configured similarly as housings disclosed herein. The delivery apparatus 342 is adapted to deliver an implant to a native heart valve.

The imaging apparatus 340 comprises an IVUS (intravascular ultrasound) imaging apparatus or catheter in examples. The imaging apparatus 340 comprises an OCT (Optical Coherence Tomography) imaging apparatus or catheter in examples.

The imaging apparatus 340 can be provided in a variety of locations. For example, the imaging apparatus 340 extends within an elongate sheath of the delivery apparatus 342. The elongate sheath comprises an outer sheath of the delivery apparatus 342 and extends coaxial with the delivery apparatus 342. In examples, the imaging apparatus 340 extends external to the delivery apparatus 342. The imaging apparatus 340 protrudes distally from a capsule, distal end, or other portion of a delivery apparatus 342 during imaging. The capsule 349, for example, includes the distal end 347 of the elongate sheath. Other locations for the imaging apparatus 340 can be utilized.

In examples, the imaging apparatus 340 is positioned interior of a prosthetic valve 344 upon a prosthetic valve being deployed. The imaging apparatus 340 for example, extends distally to be positioned within the prosthetic valve 344 and to image areas surrounding the prosthetic valve 344. The imaging apparatus 340 is positioned within the flow channel of the prosthetic valve 344.

The imaging apparatus 340 is adapted to image at least a portion of a native heart valve. The imaging apparatus 340 images one or more leaflets of the native heart valve. The imaging apparatus 340 is adapted to image a capture of one or more leaflets by one or more of the anchors. For example, the anchor 17a has failed to capture the leaflet 82a. The imaging apparatus 340 can image such area to provide an image that a failed or missed capture has occurred. The anchor 17b has captured the leaflet 82b. The imaging apparatus 340 can image such area to provide an image that a capture has occurred. A user can attempt to recapture the leaflet 82a with the anchor 17a, and such recapture can be imaged by the imaging apparatus 340.

Other conditions of the area external to the delivery apparatus can be imaged with the imaging apparatus.

In examples, a prosthetic valve includes one or more windows that can allow for imaging through the prosthetic valve. For example, referring to FIG. 51, a valve body 346 includes one or more imaging windows 348 that can allow for imaging therethrough (e.g., transmission of ultrasound waves, or transmission of x-rays, among others). The windows 348 are positioned at locations of anchors in examples, to allow for imaging of capture of native valve leaflets by anchors. The imaging windows 348 are spaced circumferentially from each other and positioned at end of the anchors 17. In examples, other locations of windows can be utilized as desired. The windows 348 comprise openings in a metal frame of the prosthetic valve to allow for imaging through the windows 348.

In examples, an imaging apparatus 341 is adapted to be positioned outward of a valve body 343 of a prosthetic valve 345 during deployment of the prosthetic valve 345. The imaging apparatus 341 can be positioned between the valve body 343 and one or more of the anchors 17. As such, the imaging apparatus 341 images the implantation site without imaging through the valve body 343. The imaging apparatus 341 accordingly images whether capture of one or more of the leaflets 82a, b occurs.

The signals from the imaging apparatuses are provided for view by a user during an implantation procedure, or at another time as desired.

The features of FIGS. 50A-51 may be utilized solely or in combination with any example disclosed herein. The prosthetic valves utilized may include features of other prosthetic valves disclosed herein (e.g., a prosthetic valve as disclosed with respect to FIG. 1A-3, or other forms of prosthetic valves). Other forms of prosthetic valves may be utilized in examples as desired.

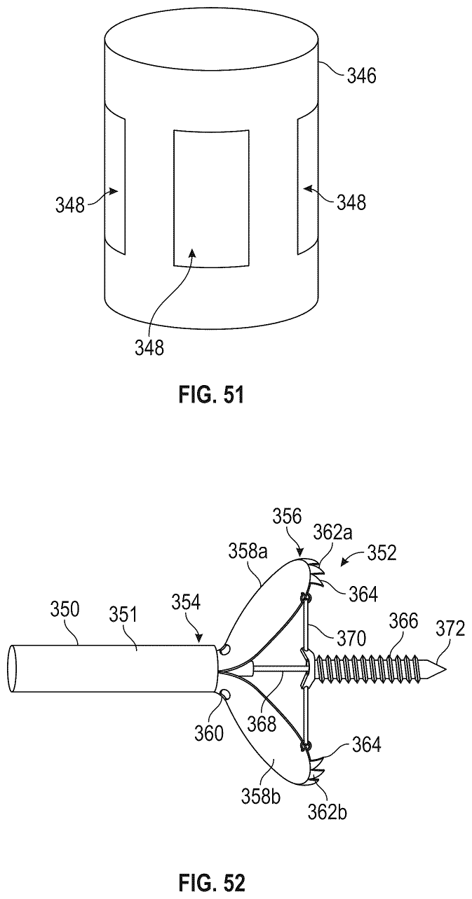

In examples, a sensor can be provided, and one or more anchors can be coupled to the sensor. The one or more anchors can be adapted to engage an interior heart wall of a chamber of a heart to anchor the sensor to the interior heart wall. Referring to FIG. 52, for example, an anchor 352 is coupled to the sensor 350. The anchor 352 is adapted to engage an interior heart wall of a chamber of a heart to anchor the sensor 350 to the interior heart wall.

The sensor 350 can have a variety of forms. The sensor 350 can be adapted to sense a condition within a patient's body. The sensor 350 can be adapted to sense a condition within a chamber of a heart. The sensor 350 can be adapted to sense a property of fluid within the chamber of the heart.

In examples, the sensor 350 comprises a pressure sensor. The pressure sensor is adapted to sense a pressure within a chamber of a heart. In examples, the sensor comprises a temperature sensor or flow sensor adapted to sense a respective temperature or flow within the chamber of the heart. Other forms of sensors can be utilized in examples. In examples, combinations of different sensors can be provided.

In examples, the sensor 350 is positioned within a support body 351. The support body 351, for example, may comprise a housing for the sensor 350 or can have another configuration as desired.

The anchor 352 is positioned at an end portion 354 of the sensor 350 or may have another position as desired. The anchor 352, for example, can be positioned at a mid portion or central portion of the sensor 350, or at an opposite end portion of the sensor 350 than shown in FIG. 52.

The anchor 352 has a variety of forms as desired. As shown in FIG. 52, the anchor 352 can include a clip 356 that includes a plurality of arms 358a, b. The arms 358a, b can be adapted to compress tissue (e.g., tissue of the interior heart wall) between the arms 358a, b to anchor to the tissue. A proximal end portion of the arms 358a, b, can be coupled together at a pivot 360. The arms 358a, b are adapted to rotate about the pivot 360 to move the respective distal end portions 362a, b of the arms towards each other and away from each other. The arms 358a, b are adapted to pivot relative to the support body 351. The arms 358a, b include one or more penetrating bodies 364 for penetrating the interior heart wall. The penetrating bodies 364 are adapted to enhance anchoring to the tissue (e.g., tissue of the interior heart wall).

The anchor 352 is adapted to penetrate the interior heart wall. In examples, the anchor 352 includes a threaded body 366 that is adapted to penetrate the interior heart wall. The threaded body 366 can be adapted to be screwed into the tissue of the interior heart wall to anchor to the interior heart wall.

In examples, a shaft 368 is provided that is adapted to slide into the threaded body 366. Upon the shaft 368 sliding distally into the threaded body 366, a pivot linkage 370 moves proximally relative to the distal end portions 362a, b of the arms to allow the distal end portions 362a, b to be drawn towards each other. Upon such movement, the distal end portions 362a, b of the arms 358a, b can be proximate a distal tip 372 of the threaded body 366. In examples, other configurations of anchors can be provided as desired.

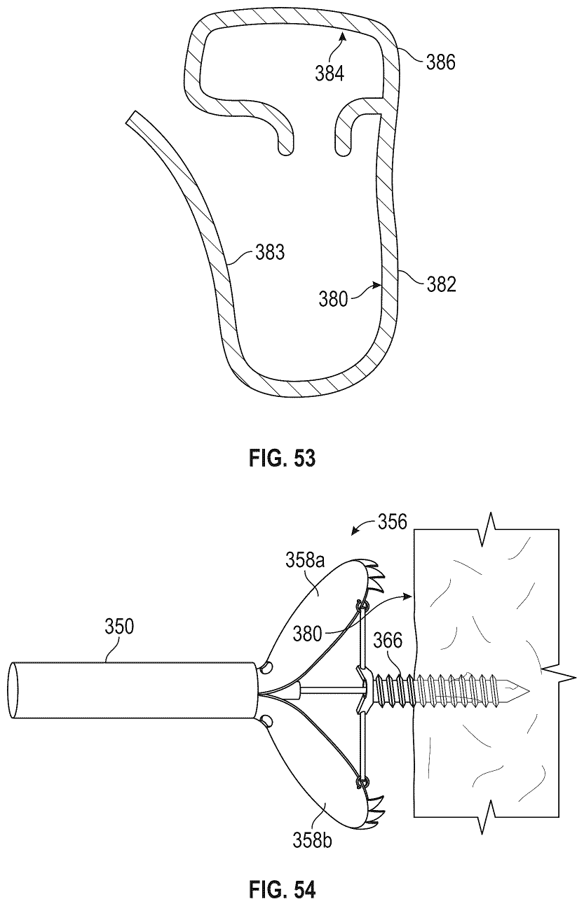

Referring to FIG. 53, the sensor 350 can be deployed to a variety of locations. A sensor 350 can be deployed to an interior heart wall 380 of a left ventricle 382 in examples. The sensor 350 can be positioned at an apex, or on the interventricular septum 383 or at another location as desired. A sensor 350 can be deployed to an interior heart wall 384 of a left atrium 386 in examples. Other interior heart walls (e.g., of a right ventricle or right atrium) can be utilized in examples.

The sensor 350 is inserted into the patient's heart and the anchor 352 can engage the tissue of the interior heart wall 380. For example, referring to FIG. 54, a portion of an anchor such as a threaded body 366 can be inserted into an interior heart wall. The threaded body 366 can be inserted to a desired distance.

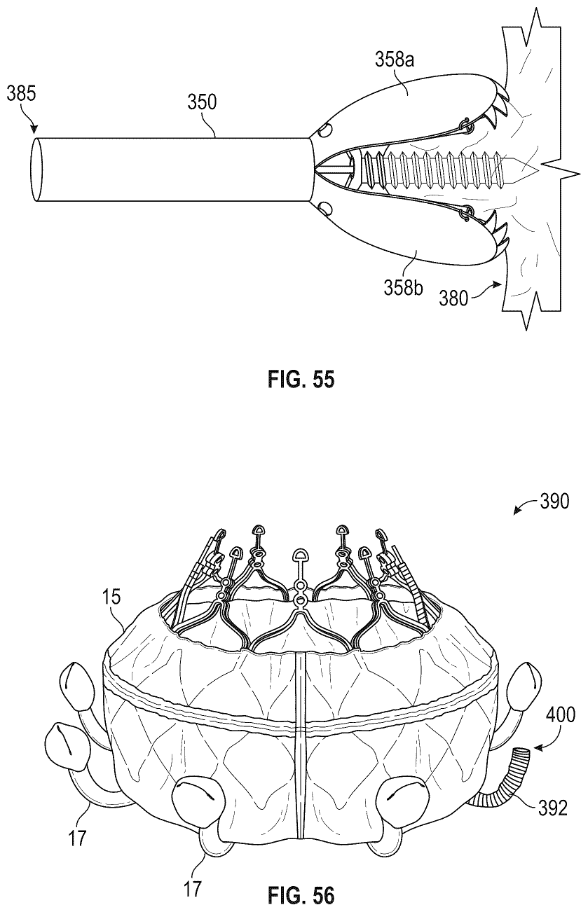

The tissue of the interior heart wall 380 can be retracted proximally. Upon retraction, the arms 358a, b of the clip 356 can be closed as shown in FIG. 55. The sensor 350 can be anchored to the interior heart wall 380.

Referring to FIG. 55, the sensor 350 is positioned within a chamber of the heart. The sensor 350 is positioned to protrude into the chamber from the interior heart wall 380, with the one or more anchors 352 engaged to the interior heart wall 380. The sensor 350 accordingly is positioned to sense a condition within the chamber. In examples the sensor 350 includes a sensing portion 385 at an end portion or other location of the sensor 350 for sensing the condition within the chamber. The sensor 350 is adapted to be positioned in a plurality of locations along the interior heart wall 380.

The sensor 350 is adapted to provide a signal indicating the sensed condition, in a similar manner as the pressure sensors discussed with respect to FIG. 33-38. For example, wireless or wired signals can be provided. A wireless transmitter as disclosed herein can be utilized to transmit signals from the sensor 350. The wireless transmitter can be provided within the support body 351 for example. A power source or other components (e.g., a processor, a memory) is positioned within the support body 351 as desired.

The configuration of the sensor 350 and the anchor 352 can be varied in examples.

The features of FIGS. 52-55 may be utilized solely or in combination with any example disclosed herein.

In examples, a least a portion of a prosthetic heart valve can comprise a pacemaker electrical conduit adapted to conduct an electrical signal for pacing a heart. Referring to FIG. 56, a prosthetic heart valve 390 can include a pacemaker electrical conduit 392. A variety of portions of the prosthetic heart valve may comprise a pacemaker electrical conduit.

As shown in FIG. 56, an anchor of the prosthetic heart valve 390 comprises a pacemaker electrical conduit 392. The prosthetic heart valve 390 includes one or more anchors 17 adapted to anchor the prosthetic heart valve 390 to a native heart valve, and the pacemaker electrical conduit 392 comprises at least a portion of one or more of the anchors. The pacemaker electrical conduit 392 is adapted to anchor the prosthetic heart valve 390 in position. The pacemaker electrical conduit 392 is adapted to anchor to a prosthetic heart valve leaflet in a similar manner as other anchors 17 of the prosthetic heart valve 390. For example, the pacemaker electrical conduit 392 is adapted to extend over a distal tip of a native valve leaflet for anchoring to the leaflet. The anchors can extend radially outward from the valve body 15. The pacemaker electrical conduit 392 can be adapted to resist a proximal force applied to the prosthetic heart valve 390.

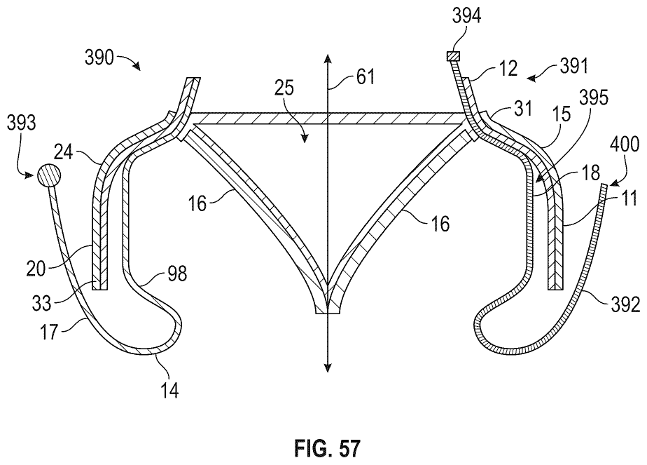

In examples, referring to FIG. 57, the prosthetic valve 390 includes a proximal end portion 391 and a distal end portion 393. The pacemaker electrical conduit 392 extends along the valve body 15 to a proximal end portion 391 of the prosthetic valve 390. The pacemaker electrical conduit 392 extends to an electrical terminal 394 positioned at the proximal end portion 391 of the prosthetic valve 390. The electrical terminal 394 is adapted for electrically connecting the pacemaker electrical conduit 392 to another electrical terminal 396 of a pacemaker 398 (as marked in FIG. 59). The electrical terminal 394 is coupled to a frame of the prosthetic valve 390 in examples.

The pacemaker electrical conduit 392 includes a portion 395 extending along the valve body 15, and a portion 400 extending radially outward from the valve body 15. The portion 400 comprises a tip portion including a tip of the pacemaker electrical conduit 392. The portion 395 extends along the frame of the valve body 15 in examples.

In examples, other portions of a prosthetic valve comprise a pacemaker electrical conduit. For example, a portion of a frame, which comprises an outer frame or an inner frame, includes a pacemaker electrical terminal. The pacemaker electrical terminal is adapted to contact a portion of a native heart valve to pace the heart.

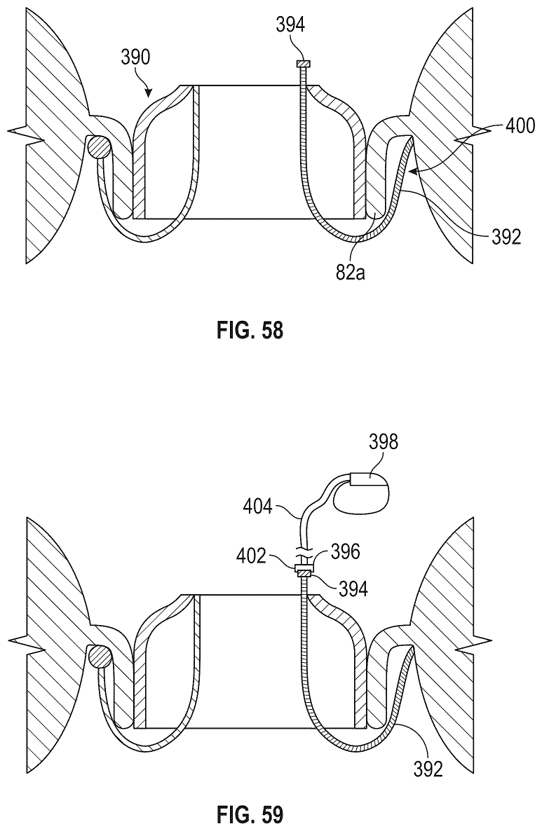

As shown in FIG. 58, upon deployment of the prosthetic valve 390, the pacemaker electrical conduit 392 is in contact with a portion of a patient's heart. For example, the pacemaker electrical conduit 392 captures a leaflet of the native heart valve. The pacemaker electrical conduit 392 hooks around the leaflet 82a to anchor to the leaflet 82a. A tip portion 400 of the pacemaker electrical conduit 392 contacts a surface of the heart, to conduct the electrical signal to the heart. For example, the tip portion 400 contacts proximate the annulus of the heart valve. The tip portion 400 contacts the annulus radially outward of the native valve leaflets in examples. In the event of a missed capture or other configuration of the pacemaker electrical conduit 392, the tip portion 400 can be positioned interior of the native valve leaflets.

The prosthetic valve 390 can be implanted and a pacemaker 398 (marked in FIG. 59) can be coupled to the electrical terminal 394 of the pacemaker electrical conduit 392. The coupling occurs as part of the implantation procedure of the prosthetic valve 390, or can occur in a separate procedure. For example, a determination can be made that a pacemaker 398 should be provided for the patient. The pacemaker 398 can be provided following the implantation of the prosthetic valve 390, with the pacemaker electrical conduit 392 available for electrical connection to the pacemaker 398. In examples, the pacemaker 398 can be integral or preconnected to the pacemaker electrical conduit 392 and can be provided for the patient at the time the prosthetic valve 390 is implanted.

Referring to FIG. 59, in a procedure, the pacemaker 398 is provided for the patient with the electrical terminal 402 of the electrical pacemaker conduit 404 being connected to the electrical terminal 394. The pacemaker 398 accordingly provides an electrical signal to the pacemaker electrical conduit 392 for pacing the heart.

In examples, other forms of connection to the pacemaker electrical conduit 392 can be made.

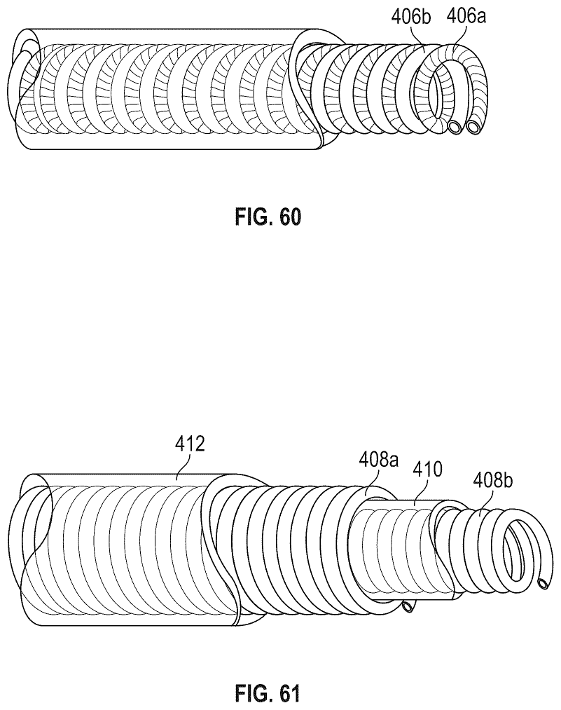

The configuration of the pacemaker electrical conduit 392 can be varied in examples. The pacemaker electrical conduit 392 includes one or more coils. For example, FIG. 60 illustrates an example in which a pacemaker electrical conduit that can be utilized includes coils 406a, b that are alternated with each other and wrapped adjacent to each other. The coils 406a, b can have the same diameter and an insulation prevents electrical shorting of the coils 406a, b. FIG. 61 illustrates a configuration in which a first coil 408a is wrapped over a second coil 408b, with an insulation layer 410 positioned between the coils 408a, b. An insulation layer 412 is wrapped over the outer coil 408a if desired. The coils 406a, b, 408a, b comprise respective cathode and anode coils as desired.

The use of a prosthetic valve having a pacemaker electrical lead provides a variety of benefits. For example, if a pacemaker is desired for a patient, then the pacemaker electrical lead is provided with the prosthetic valve for use with the pacemaker. Such a configuration can reduce a need to insert a pacemaker electrical lead through a flow channel of a prosthetic valve, which can impede operation of the prosthetic leaflets. Further, increased ease of positioning of the pacemaker electrical lead can result because the prosthetic valve provides a desired position of the pacemaker electrical lead upon implantation of the prosthetic valve. Other benefits can result.

Features of the prosthetic valve shown in FIGS. 56-59 can include features of other prosthetic valves disclosed herein (e.g., a prosthetic valve as disclosed with respect to FIG. 1A-3, or other forms of prosthetic valves). Other forms of prosthetic valves may be utilized in examples as desired.

The features of FIGS. 56-61 may be utilized solely or in combination with any example disclosed herein.

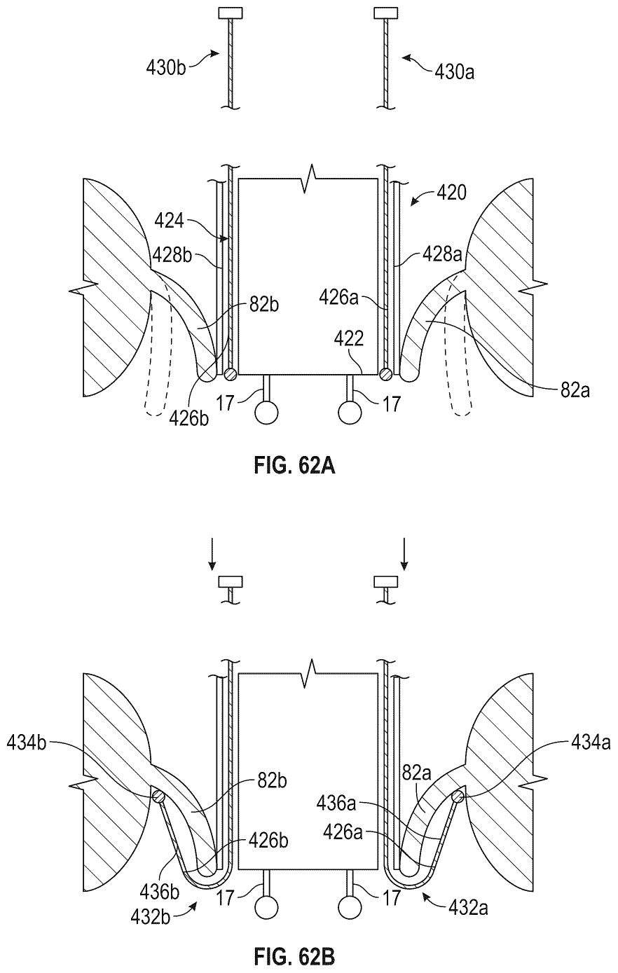

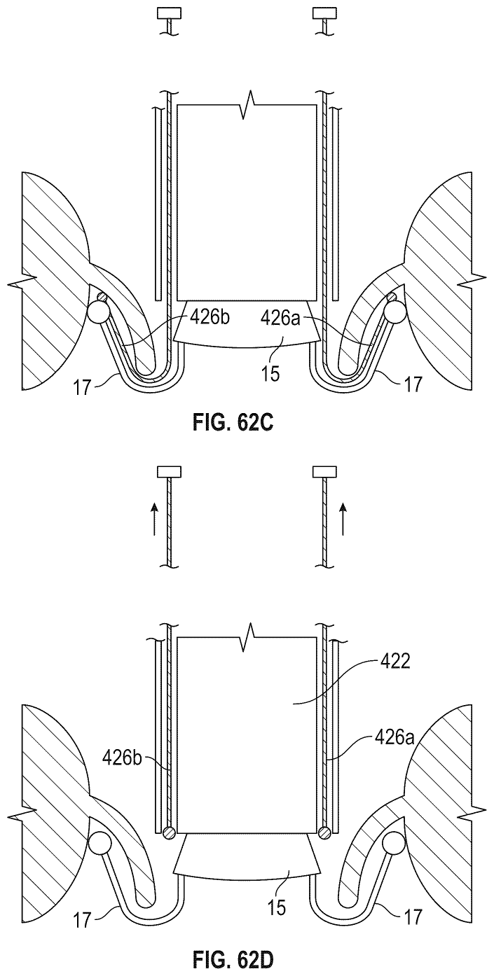

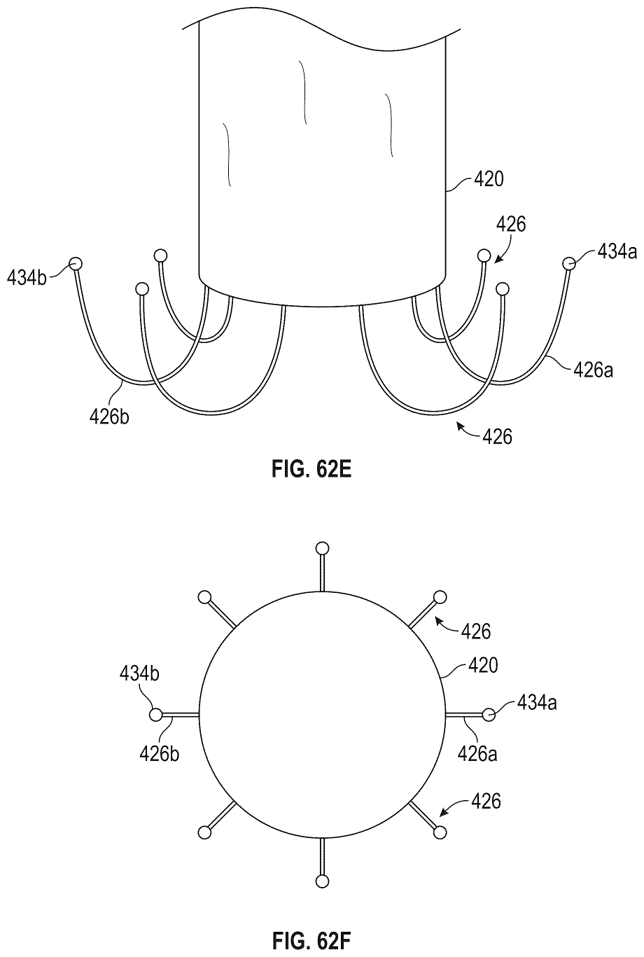

FIGS. 62A-68C illustrate implementations utilizing a retainer mechanism for retaining one or more native valve leaflets in a contracted state upon one or more anchors of a prosthetic heart valve at least partially hooking around the one or more native valve leaflets.

Referring to FIG. 62A, for example, a distal end portion of a delivery apparatus such as a delivery catheter 420 is shown approaching an implantation site. The delivery apparatus is configured similarly as other forms of delivery apparatuses disclosed herein, and includes an implant retention area in the form of a capsule 422 for retaining the prosthetic heart valve. The delivery catheter 420 is adapted to deliver the prosthetic heart valve to a native valve. The prosthetic heart valve can be configured similarly as other forms of prosthetic heart valves disclosed herein. For example, the anchors 17 of the prosthetic heart valve (similar to the anchors 17 shown in FIGS. 1A-1C and 4A) are shown in a linearized configuration and extending from the distal end of the capsule 422.

The retainer mechanism 424 includes one or more arms 426a, b that are adapted to hook around the native valve leaflets 82a, b. The arms 426a, b are shown in a retracted, unexpanded, compressed, or linearized configuration in FIG. 62A, in which the arms 426a, b extend parallel with the length of the capsule 422 and delivery catheter 420. In examples, the delivery catheter 420 includes one or more channels 428a, b that receive the arms 426a, b and that the arms 426a, b may slide within. The arms 426a, b can slide between the retracted, unexpanded, compressed, or linearized configuration shown in FIG. 62A and an advanced, expanded, deployed, or flared configuration as shown in FIG. 62B. In examples, proximal portions 430a, b of the retainer mechanism 424 or arms 426a, b are controllable or adjustable at the proximal portion of the delivery apparatus to allow for control of the one or more arms 426a, b. The proximal portions 430a, b can be advanced to advance the arms 426a, b and can be retracted to retract the arms 426a, b. The proximal portions 430a, b can be independently controllable to independently control each arm 426a, b or can be controlled in combination as a group.

The arms 426a, b in the retracted, unexpanded, compressed, or linearized configuration shown in FIG. 62A are in a configuration for advancement to an implantation site and extend along the capsule 422 and delivery catheter 420. The arms 426a, b can be advanced to move to the advanced, expanded, deployed, or flared configuration as shown in FIG. 62B.

Referring to FIG. 62B, the arms 426a, b are shown in the advanced, expanded, deployed, or flared configuration. The arms 426a, b protrude radially outward from the delivery catheter 420 and capsule 422. The arms 426a, b form hook shapes with a respective bend portion or curved portion 432a, b and a tip 434a, b. A respective elongate portion 436a, b extends between the curved portion 432a, b and the tip 434a, b. The arms 426a, b are adapted to hook around the native valve leaflets 82a, b with the tips 434a, b and elongate portions 436a, b positioned radially outward of the respective leaflets 82a, b. The tips of the respective leaflets 82a, b can be positioned within the curved portion 432a, b.

A plurality of the arms 426a, b can be circumferentially spaced from each other, and each protrude radially outward from the capsule 422 and delivery catheter 420. FIG. 62E, for example, illustrates an arrangement of arms 426 protruding radially outward from the delivery catheter 420. FIG. 62F illustrates a top cross-sectional view. A greater or lesser number of arms 426 can be utilized as desired.

The arms 426 can comprise flexible bodies that are shape set into the hooked configuration. The arms 426 can be made of a shape memory material (e.g., nitinol) or another form of material as desired. The arms 426 can be more flexible than the material forming the anchors 17 of the prosthetic heart valve in examples. The arms 426 can be thinner than the anchors 17. Such a feature can provide for enhanced ease of deployment or threading between chordae of the native heart valve in examples.

In operation, the retainer mechanism 424 is utilized to retain the native valve leaflets 82a, b in a contracted state upon the anchors 17 at least partially hooking around the native valve leaflets 82a, b. The arms 426a, b, for example, extend radially outward to retain the native valve leaflets 82a, b in a closed or partially closed configuration (e.g., a systolic configuration for a mitral or tricuspid valve). The arms 426a, b retain the native valve leaflets 82a, b in position or against the outer surface of the delivery catheter 420 or capsule 422 in examples. Such a feature can allow for enhanced ease of capture of the native valve leaflets 82a, b by the anchors 17.

Referring to FIG. 62A, for example, the arms 426a, b are in the retracted, unexpanded, compressed, or linearized configuration and then advanced to the advanced, expanded, deployed, or flared configuration to capture the leaflets 82a, b during their systolic and diastolic motion (represented in solid and dashed lines in FIG. 62A). During the systolic motion the arms 426a, b hook around and capture the leaflets 82a, b. A configuration as shown in FIG. 62B results.

In a configuration as shown in FIG. 62B, the arms 426a, b have captured the leaflets 82a, b. The reduced motion or static position of the leaflets 82a, b allows for ease of capture by the anchors 17. FIG. 62C, for example, illustrates the anchors 17 being deployed in a similar manner as disclosed herein to anchor to the leaflets 82a, b.

At a desired time, the arms 426a, b are retracted radially inward as represented in FIG. 62D. The anchors 17 remain in position and the prosthetic heart valve can be deployed from the capsule 422 as disclosed herein. The use of the retainer mechanism provides for improved deployment of the prosthetic heart valve.

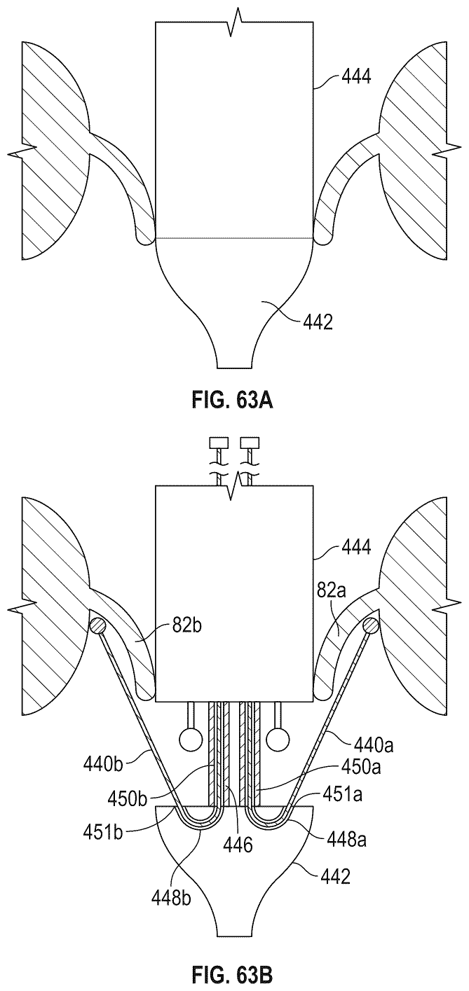

The arms 426a, b can deploy from other locations on a delivery apparatus or delivery catheter in examples. FIGS. 63A-63C illustrate an implementation in which arms 440a, b configured similarly as the arms 426a, b shown in FIGS. 62A-62F protrude radially outward from a nose body 442 of a delivery catheter 444. The nose body 442 comprises a nose cone for the delivery catheter 444. In examples, a guide wire lumen 446 couples the nose body 442 to the remainder of the delivery catheter 444. The guide wire lumen 446 passes a guide wire therethrough, which passes through the nose body 442 in examples.

Referring to FIG. 63B, the nose body 442 includes respective channels 448a, b that may pass the arms 440a, b therethrough. In examples, the channels 448a, b are curved to deflect the arms 440a, b towards the annulus positioned radially outside of the leaflets 82a, b. The delivery catheter 444 includes channels 450a, b along the guide wire lumen 446 that the arms 440a, b may pass through. The nose body 442 includes openings 451a, b that the arms 440a, b can protrude from.

The arms 440a, b operate in a similar manner as the arms 426a, b. Referring to FIG. 63C, the arms 440a, b can be retracted to the nose body 442 upon deployment of the anchors 17, as disclosed herein.

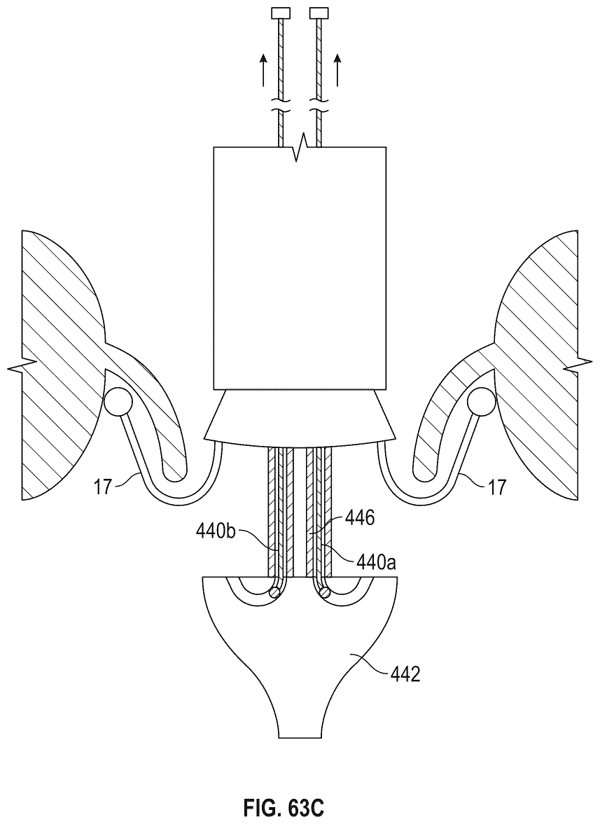

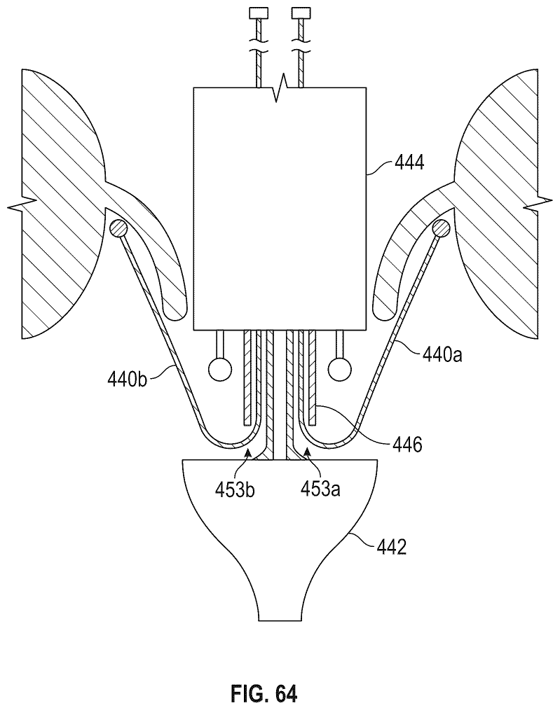

FIG. 64 illustrates a variation in which the guide wire lumen 446 includes respective openings 453a, b for the arms 440a, b to pass through. The arms 440a, b are adapted to protrude radially outward from the guide wire lumen 446.

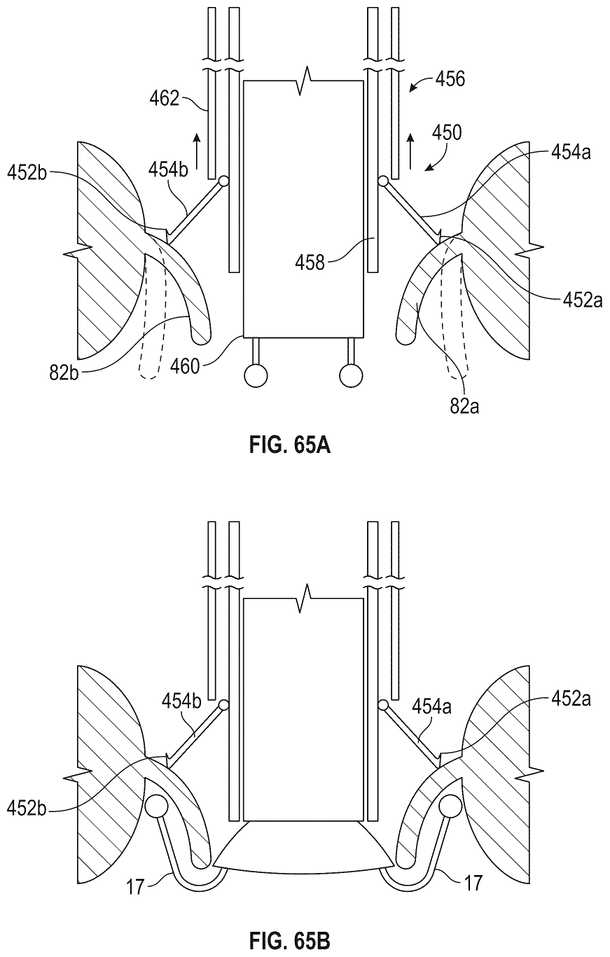

Other forms of retainer mechanisms can be utilized in examples. FIGS. 65A-65C illustrate an implementation in which a retainer mechanism 455 includes one or more barbs 452a, b for engaging the native valve leaflets 82a, b. The barbs 452a, b are positioned at ends of respective arms 454a, b that can be pivotally coupled to a portion of the delivery apparatus or delivery catheter 456 (such as a sheath 458 extending over a capsule 460 of the delivery catheter 456). The arms 454a, b are adapted to protrude radially outward from the delivery catheter 456. The arms 454a, b are circumferentially spaced from each other with the corresponding barbs 452a, b spaced from each other.

A retaining member 462 such as a sheath extends over the arms 454a, b to retain the arms 454a, b in a retracted, unexpanded, compressed, or linearized configuration. The retaining member 462 can be retracted to allow the arms 454a, b to pivot radially outward to engage the leaflets 82a, b (as represented in FIG. 65A). The arms 454a, b can operate in a similar manner as other forms of retainer mechanisms disclosed herein, and retain the leaflets 82a, b in a contracted state upon the anchors 17 at least partially hooking around the leaflets 82a, b.

FIG. 65B, for example, illustrates the barbs 452a, b engaging the leaflets 82a, b with the arms 454a, b pivoted radially outward. Upon deployment of the anchors 17, the retaining member 462 can be advanced to retract the arms 454a, b as represented in FIG. 65C.

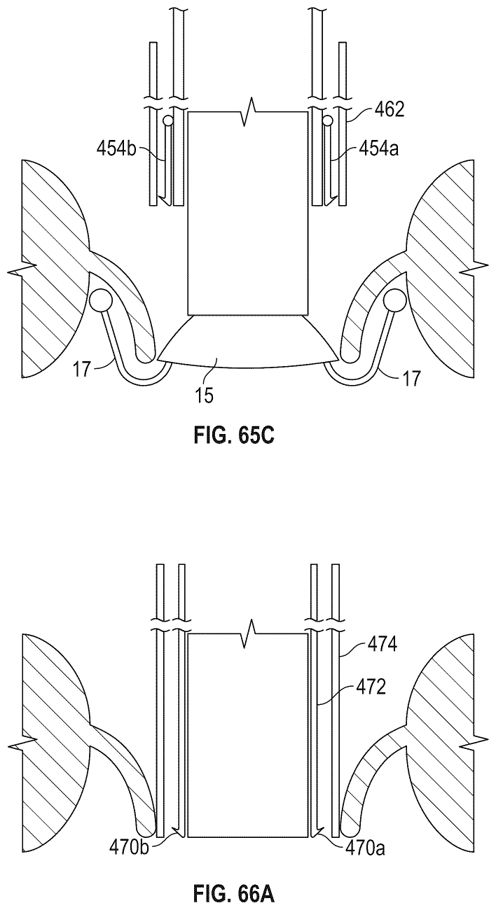

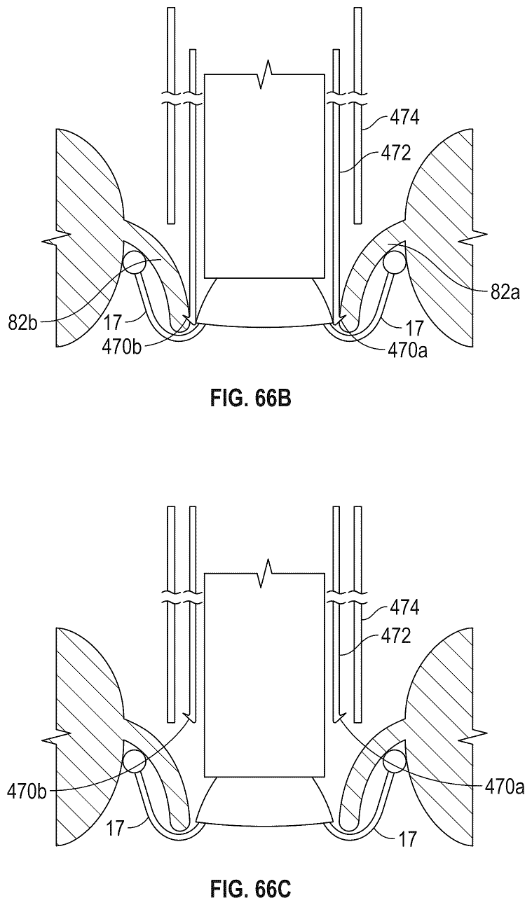

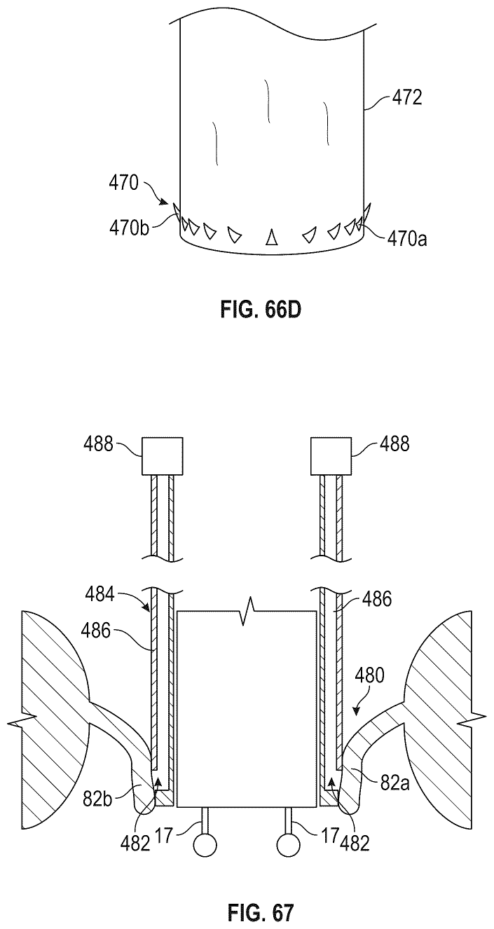

Other configurations of retainer mechanisms can be utilized in examples. FIGS. 66A-66D illustrate an example in which a plurality of barbs 470 are coupled to a sheath 472 and are circumferentially spaced from each other. The sheath 472 and barbs 470 can be covered by an outer sheath 474 (as represented in FIG. 66A) (representative barbs 470a, b are marked in FIG. 66A).

The outer sheath 474 can be retracted relative to the sheath 472 to expose the barbs 470 and allow the barbs 470a, b to engage the leaflets 82a, b as represented in FIG. 66B. The barbs 470a, b can retain the leaflets 82a, b in a contracted state upon the anchors 17 at least partially hooking around the leaflets 82a, b.

The outer sheath 474 can be advanced relative to the barbs 470a, b to cover the barbs 470a, b to release or disengage the barbs 470a, b from the leaflets 82a, b as represented in FIG. 66C.

Other configurations of retainer mechanisms can be utilized in examples. FIG. 67 illustrates an example in which a retainer mechanism 480 includes one or more suction ports 482 for applying a suction force to the native valve leaflets 82a, b to retain the leaflets 82a, b in a contracted state. The suction ports 482 are positioned on an outer surface of the delivery apparatus or delivery catheter 484. The suction ports 482 are circumferentially spaced from each other or can have another configuration in examples. One or more suction lumens 486 extend along the delivery apparatus or delivery catheter 484 to transmit the suction force to the ports 482. Suction devices 488 (e.g., a pump or syringe) are provided to produce the suction force along the suction lumens 486. The suction ports 482 can operate to retain the leaflets 82a, b in a contracted state upon the anchors 17 at least partially hooking around the leaflets 82a, b, as disclosed herein.

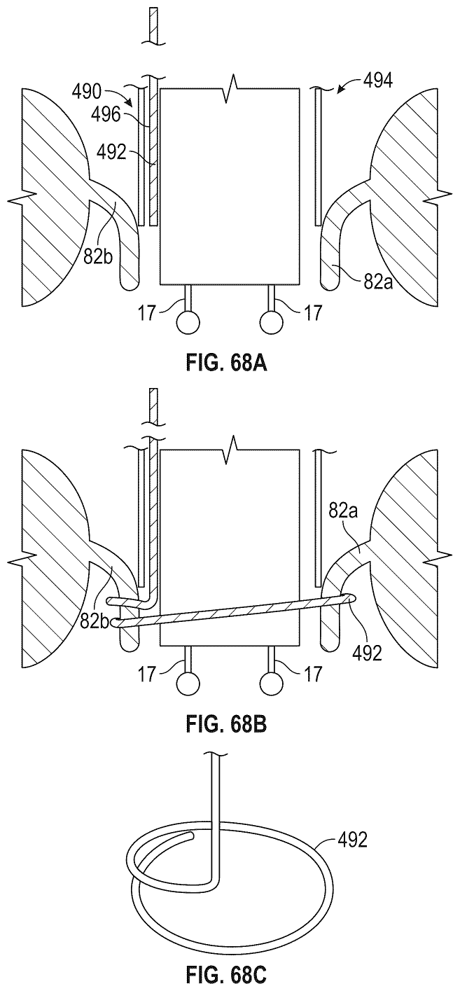

Other configurations of retainer mechanisms can be utilized in examples. FIGS. 68A-68C illustrate an implementation in which a retainer mechanism 490 includes a coil 492 for extending around a radially outward facing surface of the leaflets 82a, b. The coil 492, for example, can be adapted to deploy from the delivery catheter 494 and can protrude from a channel 496 of the delivery catheter 494 in examples. Other configurations can be utilized in examples.

The coil 492 can be shape-set (e.g., with a shape memory material such as nitinol) into a coil shape, or can be controlled (e.g., with guide wires or other mechanisms) into a coil shape. FIG. 68A illustrates the coil 492 in a retracted, unexpanded, compressed, or linearized configuration. FIG. 68B illustrates the coil 492 having been advanced (to an advanced, expanded, deployed, or coiled configuration). The coil 492 wraps about the radially outward facing surface of the leaflets 82a, b for one or more wraps as desired. FIG. 68C illustrates the coil 492 in an advanced, expanded, deployed, or coiled configuration. The coil 492 can have a flat coil shape or can have a spiral or helical shape, among other shapes.

Referring to FIG. 68B, the coil 492 operates to retain the leaflets 82a, b in a contracted state upon the anchors 17 at least partially hooking around the leaflets 82a, b, as disclosed herein. The coil 492 is retracted upon deployment of the anchors 17.

Other configurations of retainer mechanisms can be utilized in examples. For example, the indicators 90 as represented in FIGS. 7-8F comprise retainer mechanisms that remain implanted upon deployment of the prosthetic heart valve. The indicators 90 comprise flexible arms that retain the native valve leaflets in a contracted state upon the anchors 17a, b at least partially hooking around the leaflets 82a, b.

The features of FIGS. 62A-68C may be utilized solely or in combination with any example disclosed herein.

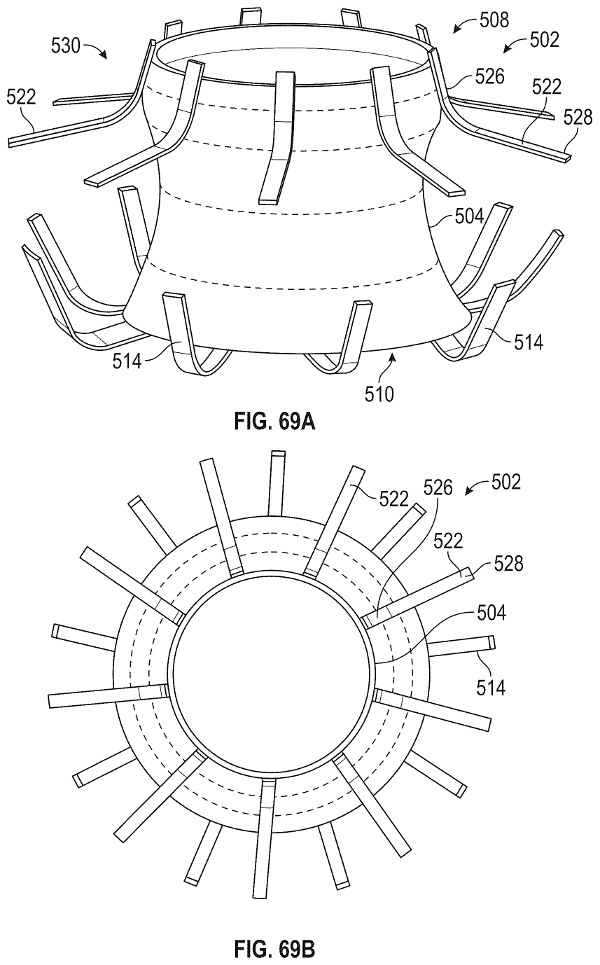

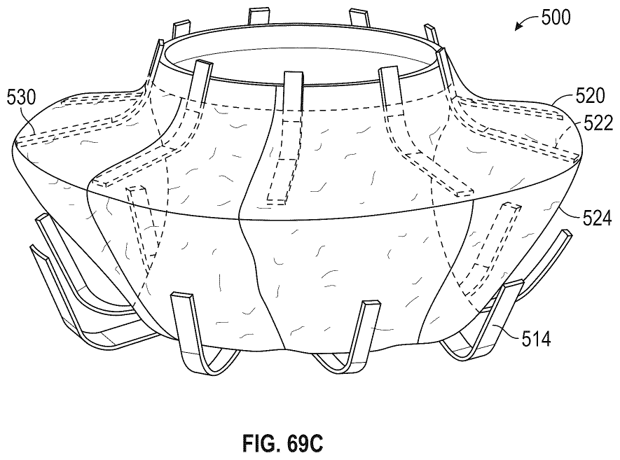

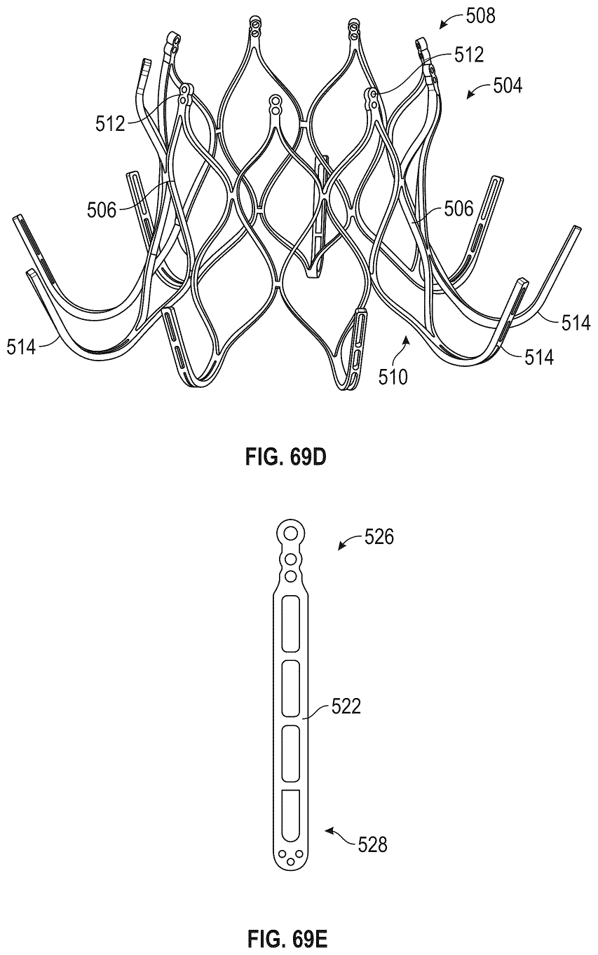

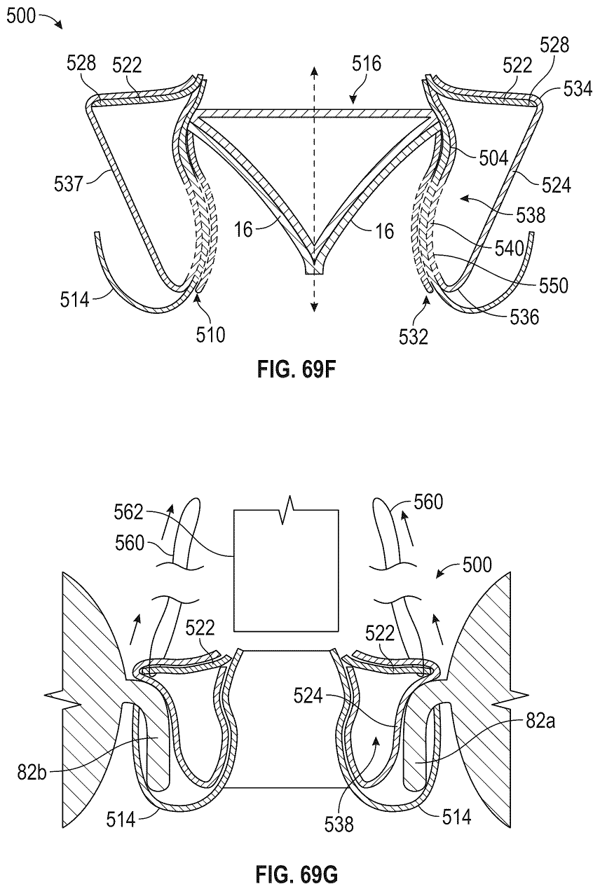

FIGS. 69A-69G illustrate an implementation of a prosthetic heart valve 500 (marked in FIGS. 69C, 69F and 69G). A frame 502 of the prosthetic heart valve 500 is represented in FIG. 69A. The frame 502 includes an inner frame 504 (shown in schematic view in FIG. 69A). A perspective view of the inner frame 504 is shown in FIG. 69D and is shown to include a plurality of struts 506. The inner frame 504 can be configured similarly as other forms of inner frames disclosed herein. The inner frame 504 includes an inflow end portion 508 and an outflow end portion 510. The inner frame 504 can be adapted to move between expanded and compressed configurations in manners discussed in regard to other forms of inner frames disclosed herein.

The proximal end portion or inflow end portion 508 of the inner frame 504 includes couplers 512 or eyelets in examples. The distal end portion or outflow end portion 510 couples to anchors 514 that can be configured similarly as the anchors 17 disclosed herein.

The inner frame 504 supports one or more prosthetic valve leaflets 16 (as marked in FIG. 69F for example) positioned within a flow channel 516 of the prosthetic heart valve 500.

The prosthetic heart valve 500 includes a sealing body 520 (marked in FIG. 69C) positioned radially outward of the inner frame 504. The sealing body 520 includes a plurality of elongate prongs 522 and a skirt 524.

Referring to FIG. 69A, the plurality of elongate prongs 522 each have a first end portion 526 coupled to the inflow end portion 508 of the inner frame 504. The elongate prongs 522 each protrude radially outward from the inner frame 504 to a second end portion 528 of the elongate prongs 522. The elongate prongs 522 comprise elongate arms protruding radially outward along radial lines extending outward from the central axis of the prosthetic heart valve 500. The first end portions 526 of the elongate prongs 522 comprise couplers for coupling with the couplers 512 of the inner frame 504. FIG. 69E, for example, illustrates a plan view of one of the elongate prongs 522 showing the first end portion 526 comprising a coupler in the form of an eyelet for suture connection with the eyelet of the inner frame 504. Other forms of couplers can be utilized in examples.

The elongate prongs 522 protrude radially outward from the first end portion 526 to form a plateau portion 530 or planar portion of the sealing body 520. The plateau portion 530 has a generally planar shape that extends outward from the inner frame 504 as a disk.

The elongate prongs 522 are circumferentially spaced from each other about the inflow end portion 508 of the inner frame 504. The elongate prongs 522 extend outward to the second end portions 528 such that the second end portions 528 form the outermost portion of the plateau portion 530. The elongate prongs 522 extend to be positioned circumferentially between adjacent anchors 514 (as shown in the top view of FIG. 69B) or can be in radial alignment with the adjacent anchors 514 in examples.

The elongate prongs 522 are deflectable in an axial dimension of the prosthetic heart valve 500. For example, an axial force upon the second end portions 528 of the elongate prongs 522 deflects the second end portions 528 about the first end portions 526.

The skirt 524 (marked in FIG. 69C) is suspended between the second end portions 528 of the elongate prongs 522 and an outflow end portion 532 of the prosthetic heart valve 500 (marked in FIG. 69F). The outflow end portion 532 of the prosthetic heart valve 500 comprises the outflow end portion 510 of the inner frame 504. Referring to the cross-sectional view of FIG. 69F, for example, the skirt 524 includes a first portion 534 positioned at the second end portions 528 of the elongate prongs 522 and includes a second portion 536 coupled to the inner frame 504. The intermediate portion 537 is suspended between the first portion 534 and the second portion 536. The skirt 524 bounds a pocket 538 positioned between the skirt 524 and the inner frame 504. The pocket 538 has an annular or ring shape about the inner frame 504. An inner portion 540 of the skirt 524 is positioned inward of the intermediate portion 537.

The skirt 524 extends along the elongate prongs 522 from the first end portion 526 to the second end portions 528 of the prongs 522 and then extends to couple to the distal end portion or outflow end portion of the inner frame 504. The inner portion 540 of the skirt 524 covers a surface of the inner frame 504.

Referring to FIG. 69F, the inner portion 540 of the skirt 524 includes a plurality of apertures 550 that allow blood to enter the pocket 538. The blood flows through the inner frame 504 and the apertures 550 to enter the pocket 538. In examples, the blood forms a clot within the pocket 538 to provide for improved sealing with the native valve annulus.

The anchors 514 are configured similarly as other forms of anchors disclosed herein. The anchors 514 can anchor the prosthetic valve to the native valve by capturing a native valve leaflet. The anchors 514 are coupled to the outflow end portion 510 of the inner frame 504 and protrude radially outward from the outflow end portion 510. The anchors 514 are adapted to hook around the native valve leaflets to anchor the prosthetic heart valve 500 to the native valve. FIG. 69G, for example, illustrates a deployed configuration of the prosthetic heart valve 500.

Referring to FIG. 69G, the skirt 524 contacts and seals against the tissue of the native valve (e.g., the leaflets or annulus). The skirt 524 comprises a conformable body that contours to a shape of the local annulus. The elongate prongs 522 comprise atrial anchors that impede ventricular movement of the prosthetic heart valve 500. The anchors 514 comprise ventricular anchors that impede atrial movement of the prosthetic heart valve 500. The pocket 538 fills with blood and clots to stabilize the prosthetic heart valve 500 within the native valve annulus.

In examples, upon deployment, the elongate prongs 522 are deflectable using one or more tethers 560. The tethers 560 can be individually actuatable to allow for independent retraction of any of the elongate prongs 522. The retraction allows for repositioning or reseating of the elongate prongs 522 upon deployment. The tethers 560 comprise a portion of a delivery apparatus such as a delivery catheter 562 and can be removed following implantation.

The configuration of the sealing body 520 allows for compliance with the shape of the native valve annulus and improved sealing. Further, reduced radial force can be exerted as opposed to a configuration of a sealing body having a rigid outer frame.

The features of FIGS. 69A-69G can be utilized solely or in combination with any example disclosed herein.

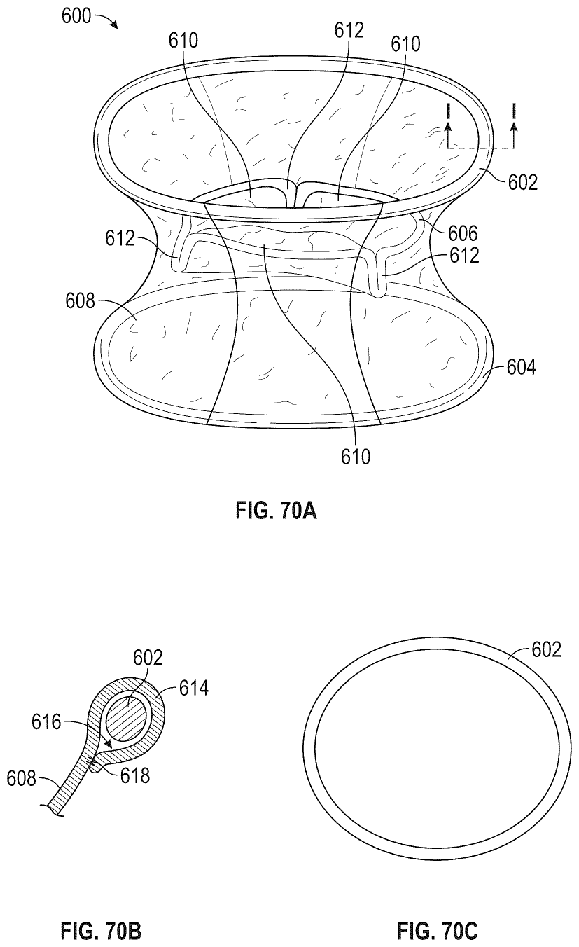

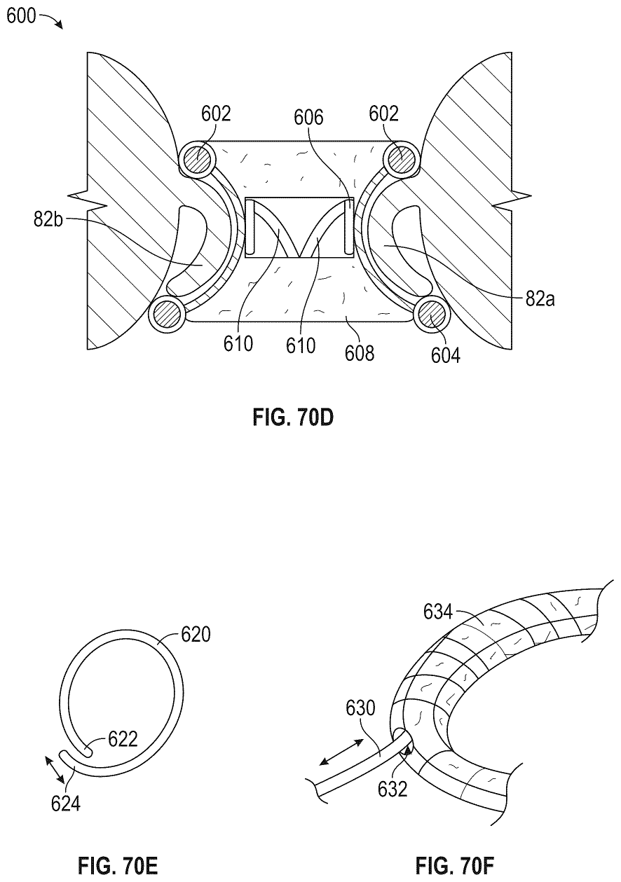

FIGS. 70A-73B illustrate implementations of prosthetic heart valves including one or more prosthetic valve leaflets and a support structure for supporting the one or more prosthetic valve leaflets and including at least one ring coupled to a skirt, with the skirt or the at least one ring being adapted to seal with at least a portion of the native heart valve.

Referring to FIG. 70A for example, an implementation of a prosthetic heart valve 600 is illustrated, including a first ring 602, a second ring 604, and a third ring 606. A skirt 608 is coupled to the rings 602, 604, 606 and extends between the first ring 602 and the second ring 604. The skirt 608 forms a sheath between the first ring 602 and the second ring 604.

The third ring 606 comprises a support body for supporting the prosthetic valve leaflets 610. The third ring 606 or support body is coupled to the prosthetic valve leaflets 610 and is coupled to the first ring 602 and the second ring 604 with the skirt 608. The third ring 606 is shaped to support the prosthetic valve leaflets 610 and can include commissure supports 612 for supporting the commissures of the prosthetic valve leaflets 610. The commissure supports 612 comprise vertical bodies sutured to the prosthetic valve leaflets 610 or can have another configuration in examples.

The first ring 602 is positioned at an inflow end portion of the prosthetic heart valve 600. The second ring 604 is positioned at an outflow end portion of the prosthetic heart valve 600. Each of the rings can be compliant and flexible to allow the rings to be compressed into a compressed state for deployment. Each ring can be adapted to vary in shape. Each ring can be biased to expand radially outward to expand upon deployment. For example, each ring can be made of a shape memory material (such as nitinol or another form of shape memory material).

The skirt 608 couples to the first ring 602 and the second ring 604 by overlapping a portion of the first ring 602 and/or second ring 604. For example, referring to the cross-sectional view of FIG. 70B (along line I-I in FIG. 70A), an end portion 614 of the skirt 608 overlaps the ring 602 to couple to the ring 602. A channel 616 is formed by the end portion 614 of the skirt 608 for receiving the ring 602. The skirt 608 can couple to itself via sutures 618 or another form of coupler in examples to form the channel 616. FIG. 70C illustrates the ring 602 in isolation from the skirt 608.

FIG. 70D illustrates the prosthetic heart valve 600 upon deployment. The first ring 602 is positioned on an atrial side of the annulus and the second ring 604 is positioned on a ventricular side of the annulus. The third ring 606 is supported by the skirt 608 between the first ring 602 and the second ring 604. The rings 602, 604, 606, and the skirt 608 seal with a portion of the native valve. The rings 602, 604, 606 and skirt 608 form a compliant body that conforms to the shape of the native valve for improved sealing.

Variations in the features of the prosthetic heart valve 600 can be provided. FIG. 70E, for example, illustrates a variation in the ring of the first ring 602 or second ring 604, in which the ring 620 includes a first end 622 and a second end 624 and the first end 622 is adapted to slide relative to the second end 624 to vary a diameter of the ring 620. The ring 620 may have improved ability to expand and contract for expansion and compression of the ring as desired, with the ends 622, 624 free to slide relative to each other.

FIG. 70F illustrates a variation in the ring of the first ring 602 or second ring 604, in which a ring 630 is passed through a channel 632 of the skirt 634 in vivo to control a diameter of the ring 630 in vivo. Either of the rings 602, 604 can have an adjustable diameter in vivo by controlling a length of the material comprising the ring within the channel 632.

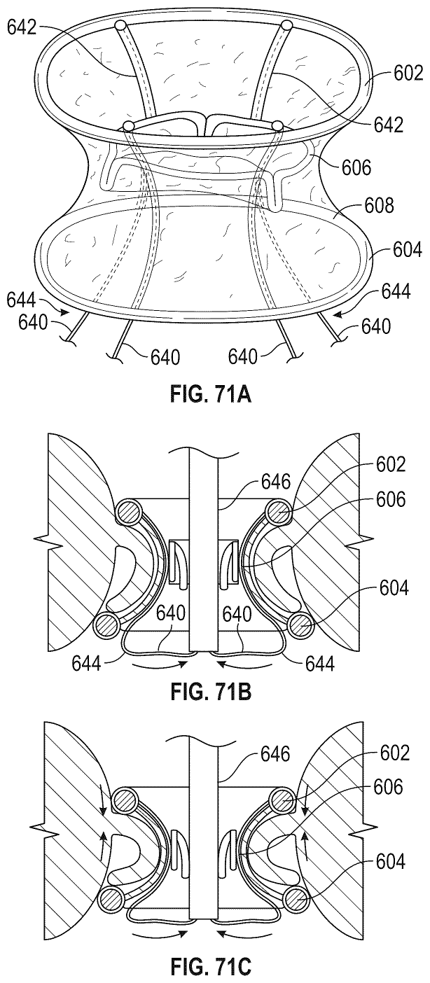

FIGS. 71A-71C illustrate a variation in which one or more tethers 640 are utilized for compressing the first ring 602 and the second ring 604 together. The tethers 640 extend along channels 642 extending axially along the skirt 608. Ends of the tethers 640 are coupled to the first ring 602. The opposite end portions 644 of the tethers 640 are pulled or retracted to draw the rings 602, 604 towards each other axially.

FIG. 71B, for example, illustrates a deployed configuration. The opposite end portions 644 of the tethers 640 pass to a sheath 646 and can be retracted through the sheath 646. Axial compression results as shown in FIG. 71C. The tethers 640 can be locked and/or cut in position to secure axial compression at the implantation site. An axial compression can be preferable to a radial expansion to reduce the possibility of conduction disturbance or other adverse effects upon the annulus.

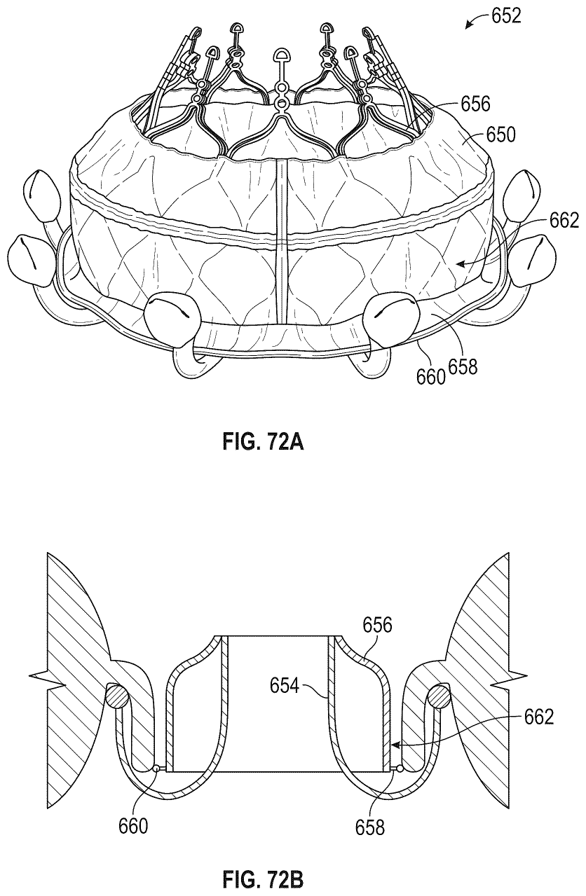

FIGS. 72A and 72B illustrate a configuration in which the support structure 650 of the prosthetic heart valve 652 includes an inner frame 654 and an outer frame 656 positioned radially outward of the inner frame 654. The prosthetic heart valve 652 can be configured similarly as the prosthetic heart valve 10 shown in FIGS. 1A-2, yet can include the skirt 658 forming a disk extending radially outward from the outer frame 656. The skirt 658 is supported at an outer periphery of the disk by a ring 660, which can be configured similarly as other examples of rings disclosed herein. The skirt 658 protrudes radially outward from the distal end portion 662 or outflow end portion of the outer frame 656 and provides a seal with the native valve upon deployment, as represented in FIG. 72B. The ring 660 and/or skirt 658 may press against the native valve (e.g., the leaflets or annulus) to provide an enhanced seal upon deployment.

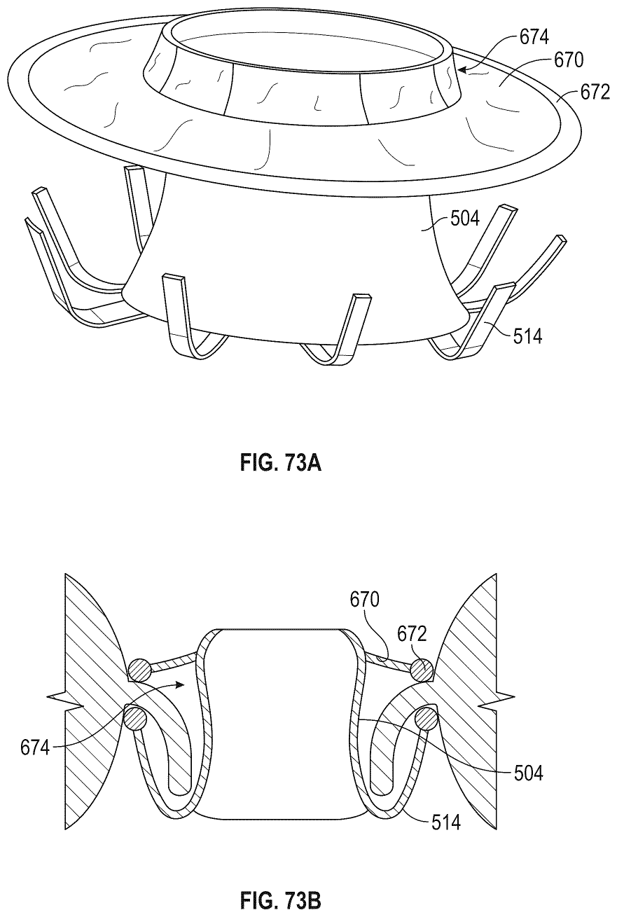

FIG. 73A illustrates a variation in which a skirt 670 forms a disk extending radially outward from an inner frame 504 as shown in FIG. 69A. The skirt 670 is supported at an outer periphery by a ring 672. The skirt 670 protrudes radially outward from the proximal end portion 674 or inflow end portion of the inner frame 504. The skirt 670 is adapted to be positioned on an inflow side of a native valve as represented in FIG. 73B for example. The ring 672 and/or skirt 670 seal against the valve annulus or native valve leaflets on an atrial side of the native valve.

The features of FIGS. 70A-73B may be utilized solely or in combination with any example disclosed herein.

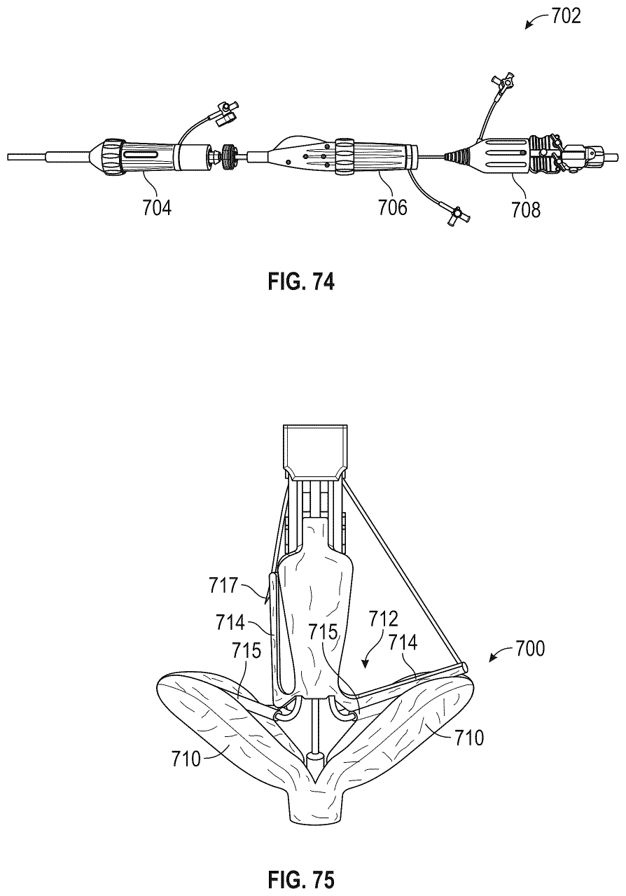

FIGS. 74-83 illustrate an implementation of a sensor system according to examples herein. The sensor system may be utilized with various other systems, apparatuses, or methods disclosed herein. FIGS. 74-83 illustrate an implementation in which the sensor system includes a prosthetic heart implant in the form of a clip 700, yet other forms of prosthetic heart implants (e.g., prosthetic heart valves) may utilize features of the sensor system.

FIG. 74 illustrates a side view of delivery system 702 that can be utilized with the clip 700. The delivery system 702 can include a plurality of catheters including a guide catheter 704, a steering catheter 706, and/or an implant deployment catheter 708. The implant deployment catheter 708 can pass through the steering catheter 706. The steering catheter 706 can pass through the guide catheter 704. The steering catheter 706 can be utilized to steer the clip 700 into a desired position. The implant deployment catheter 708 can be utilized to position the clip 700 and release the clip 700 at a desired time. Variations in the configuration of the delivery system can be utilized in examples.

FIG. 75 illustrates a side view of the clip 700. The clip 700 includes a plurality of arms or paddles 710 that can be pivotally coupled to each other. A pivot linkage 712 can be provided that can cause the arms or paddles 710 to open or close in a similar manner as the pivot linkage 370. The clip 700 includes one or more engagement arms including a first set or upper set of engagement arms 714 and a second set of lower set of engagement arms 715. The respective sets of engagement arms 714, 715 include barbs 717 or other engagement features for grasping tissue of a leaflet therebetween. Features of a clip and/or delivery system that can be utilized are disclosed in WIPO Publication No. WO/2023/003755, titled “Sensing Heart Valve Repair Devices,” which published on Jan. 26, 2023, and is a publication of International Application No. PCT/US2022/037176; WIPO Publication No. WO/2023/004098, titled “Heart Valve Repair Devices,” which published on Jan. 26, 2023, and is a publication of International Application No. PCT/US2022/037983; and International Application No. PCT/US2023/028329, filed Jul. 21, 2023; the entire contents of each of the foregoing being incorporated by reference herein for all purposes.

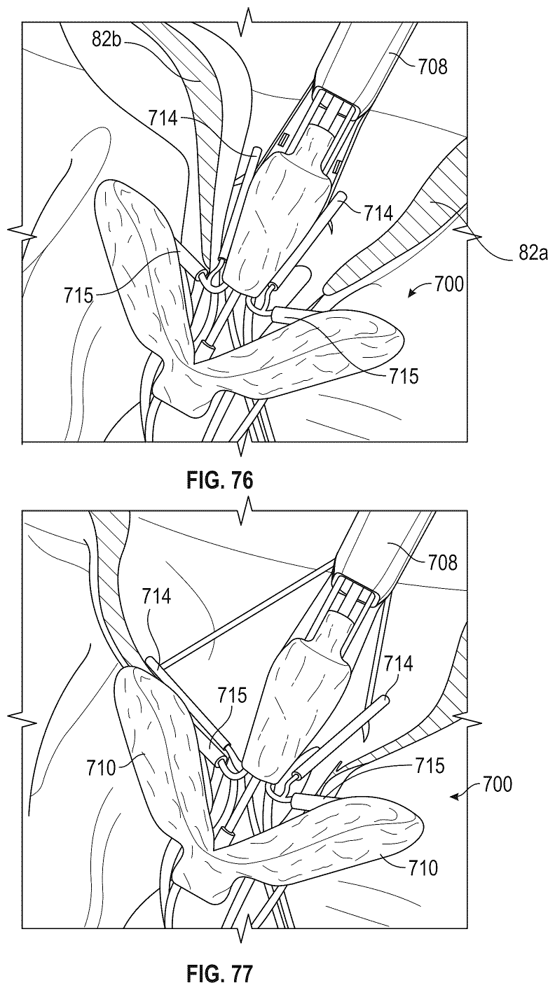

The sets of engagement arms 714, 715 and the paddles 710 are controllable to control engagement of heart valve leaflets. FIGS. 76-78 illustrate an exemplary deployment sequence. FIG. 76, for example, illustrates the clip 700 being positioned between two heart valve leaflets 82a, b with the engagement arms 714, 715 and paddles 710 in an opened configuration. Portions of the heart valve leaflets 82a, b fit between the engagement arms 714, 715. The clip 700 is coupled to the implant deployment catheter 708. At least one of the upper set of engagement arms 714 can be closed to grip a portion of a heart leaflet with one of the lower set of engagement arms 715. FIG. 77, for example, illustrates this configuration. At a desired time, the other engagement arms 714, 715 are closed to grip a portion of the other heart leaflet therebetween. The paddles 710 can be closed.

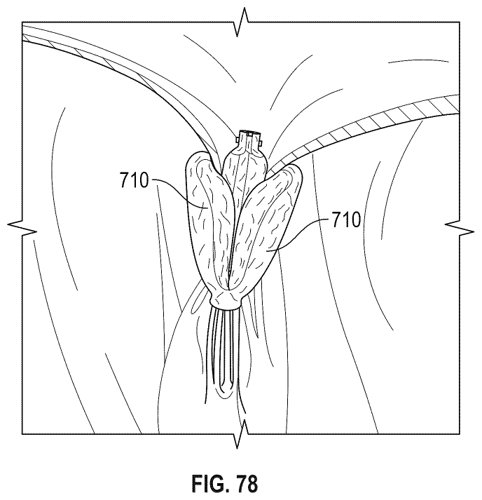

With the clip 700 in position, the implant deployment catheter 708 is removed as represented in FIG. 78. The clip 700 has clipped together the heart leaflets. The heart leaflets are clipped together to reduce regurgitation or other maladies of the valve.

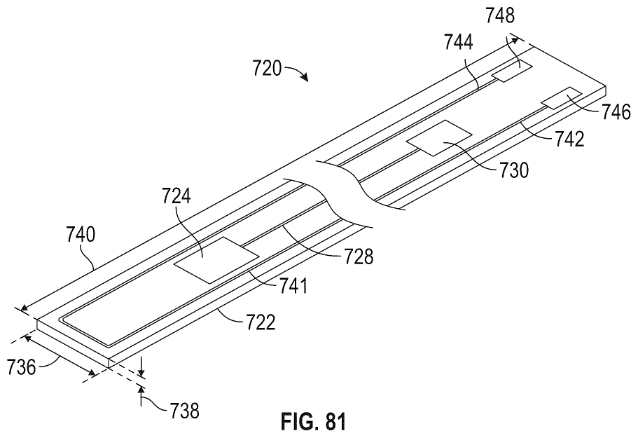

In examples, one or more sensor bodies 720 (marked in FIG. 80) are incorporated into the system. A perspective view of a sensor body 720 is shown in FIG. 81. Referring to FIG. 81, the sensor body 720 includes a substrate 722 and a sensor 724 positioned on the substrate 722.

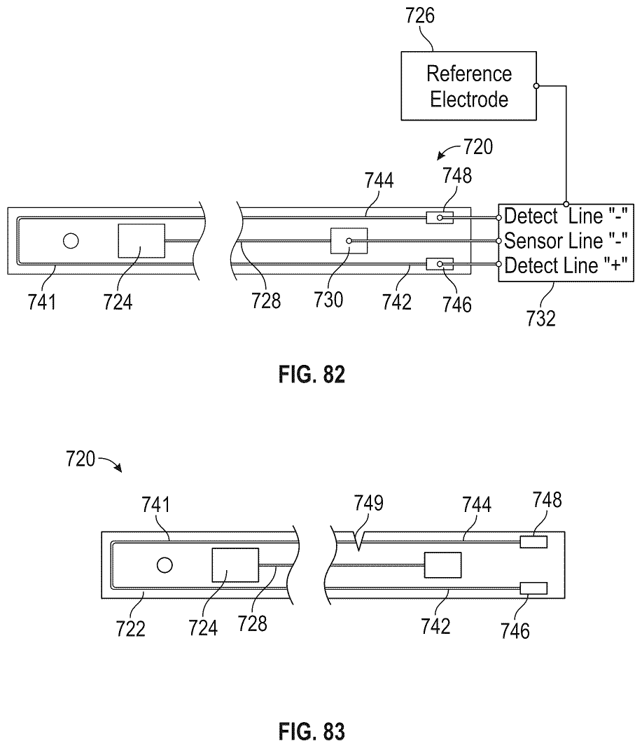

The sensor 724 is adapted to detect a condition of the prosthetic heart implant (e.g., the clip 700). The sensor 724 is adapted to detect contact between a portion of the clip 700 and the heart leaflets. For example, the sensor 724 comprises an electrode, with a second reference electrode 726 (marked in FIG. 82) coupled to a portion of the patient's body. The electrical signal between the sensor 724 and the reference electrode 726 upon contact between the sensor 724 and tissue (e.g., the heart valve leaflets) indicates contact between the clip 700 and the heart valve leaflets. The signal from the sensor 724 passes along an electrical trace 728 to an electrical terminal 730. The electrical terminal 730 couples to a controller 732 (marked in FIG. 82) that determines if contact has occurred due to the signal from the sensor 724. The controller 732 comprises a processor as disclosed herein, or comprises other forms of controllers for detecting a signal from the sensor 724. As shown in FIG. 82, the reference electrode 726 can be electrically coupled to the controller 732.

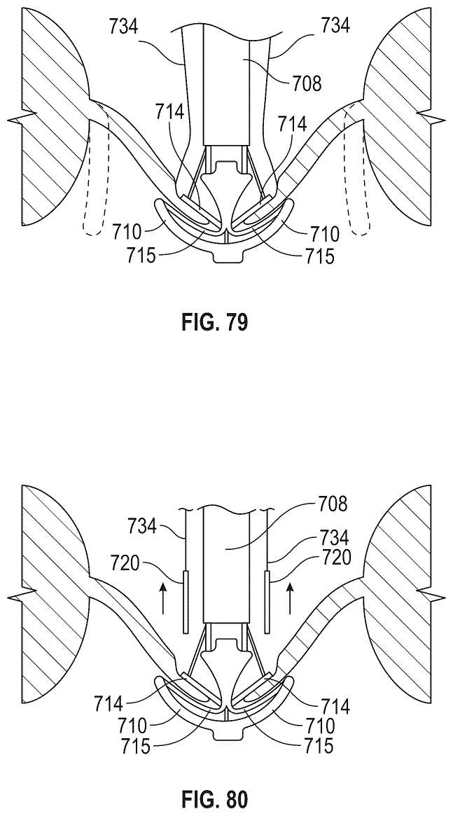

The sensor body 720 and sensor 724 are positioned to detect the contact between the clip 700 and the heart valve leaflets. For example, referring to FIGS. 79 and 80, the sensor bodies 720 and sensors 724 are positioned in one or more of the engagement arms 714, 715 or paddles 710 for detecting contact between the respective engagement arms 714, 715 or paddles 710 and the heart leaflet tissue. The sensors 724 are positioned to contact the heart valve tissue for the detection of the contact.

In examples, the sensor bodies 720 are adapted to be extracted from a portion of the clip 700 following implantation. Referring to FIG. 80, the sensor bodies 720 are shown being removed from the clip 700 upon implantation, such that the sensor bodies 720 do not remain implanted within the patient's body with the clip 700 implanted. A tension force is applied to the sensor bodies 720 with tethers 734 or a tension force can be applied directly to the sensor bodies 720 for extraction (e.g., at a proximal end portion of the delivery system). The sensor bodies 720 can be retracted into a catheter of the delivery system at a desired time.

Referring to FIG. 81, the substrate 722 comprises a circuit board. The circuit board is a flexible circuit board that the sensor 724 and electrical trace 728 are printed upon. The flexible circuit board is a laminated structure including multiple layers of material, including the conductive layers of the sensor 724 and the electrical trace 728. The sensor body 720 is configured as a strip of material, and has relatively narrow dimensions (e.g., a width 736 of less than 1 millimeter or less than 0.5 millimeters, a thickness 738 of less than 0.5 millimeter or less than 0.2 millimeters). The sensor body 720 is relatively long (with a length 740 of greater than 100 centimeters, or greater than 150 centimeters), which allows for the proximal end of the sensor body 720 to be pulled through the delivery system following implantation.

A concern with extraction of the sensor body 720 is undue force upon the sensor body 720, which can be produced during the extraction process. The tension upon the sensor body 720 may produce force that may result in damage to the sensor body 720 (e.g., a partial or full tear). Such a result would be undesirable as fragments of the sensor body 720 may be free within the patient's body or otherwise damage to the sensor body 720 upon other forms of force may result.

As such, it is desirable to be able to detect force applied to the sensor body 720 and the substrate 722. The force can indicate a full or partial tear of the substrate 722 or other undesirable forces upon the substrate 722 (e.g., an undue extraction force upon the substrate 722).

Referring to FIG. 81, an electrical detection trace 741 is positioned on the substrate 722. The electrical detection trace 741 is adapted to detect a force applied to the substrate 722. The electrical detection trace 741 comprises a conductive trace on the substrate 722 that extends along the perimeter of the substrate 722. A loop shape is formed, with a first end 742 and a second end 744. A first electrical terminal 746 is at the first end 742 and a second electrical terminal 748 is at the second end 744. The electrical terminals are adapted to electrically couple to a controller 732 as indicated in FIG. 82. The controller 732 produces a current along the electrical detection trace 741 and monitors the current or resistance maintained along the electrical detection trace 741 upon extraction.

FIG. 83, for example, illustrates a partial tear 749 in the substrate 722 during an extraction procedure. The tear breaks the electrical detection trace 741, thus increasing the resistance of the electrical detection trace 741 and/or reducing the current applied along the electrical detection trace 741. The controller 732 detects this variation. The controller 732 produces an indication, using any form of indicator device disclosed herein. The technician is aware of damage to the substrate 722 and can modify the procedure by ceasing extraction of the sensor body 720 and fully removing the clip 700 and delivery system.

The electrical detection trace 741 is adapted to detect a partial or full tear of the substrate 722 upon in vivo extraction of the sensor body 720 from the clip 700.

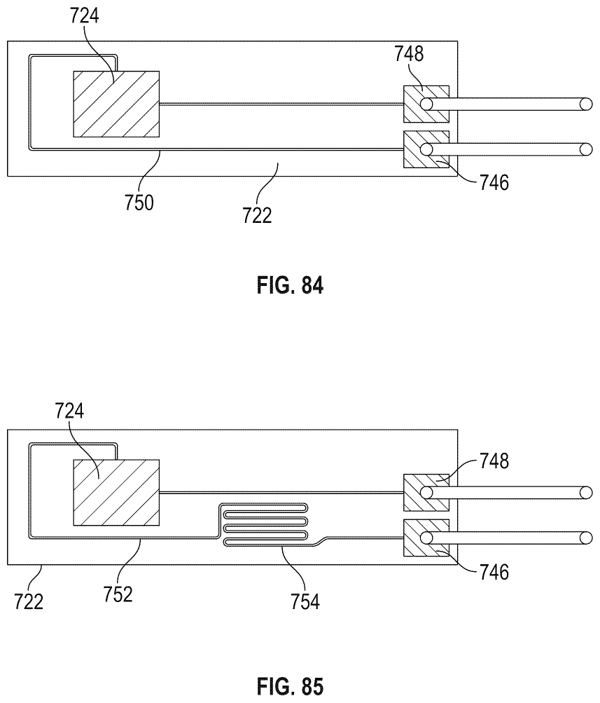

Other forms of electrical detection traces can be utilized in examples. FIG. 84, for example, illustrates a variation in which an electrical detection trace 750 electrically conducts through the sensor 724. As such, the electrical detection trace 750 electrically passes through the sensor 724 to reduce the size of the electrical detection trace 750 in comparison with a configuration as shown in FIG. 83. The controller 732 can be modified to account for the electrical detection trace 750 and the sensor 724 sharing a same electrical trace line.

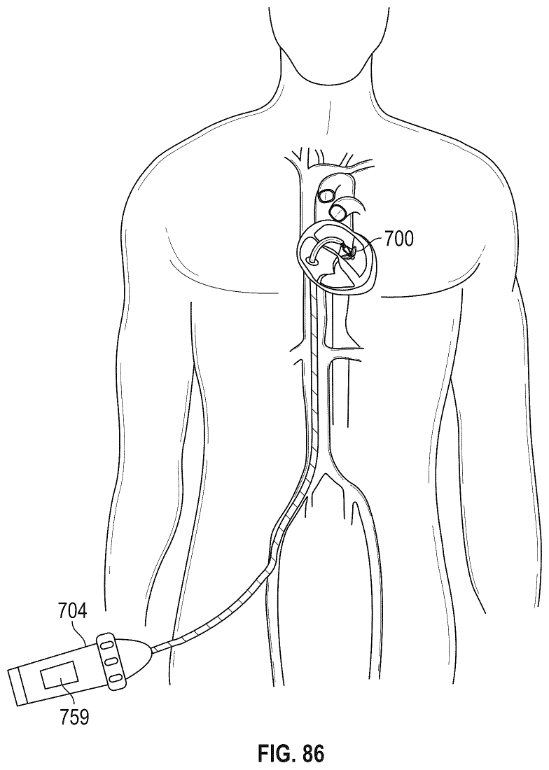

FIG. 85 illustrates a variation in which the electrical detection trace 752 includes a strain gauge 754 on the substrate 722. The strain gauge 754 produces an electrical signal indicating an amount of force applied to the substrate 722. The signal is received by the controller 732. The controller 732 is adapted to produce an output on an indicator device 759 (such as a display screen on one of the catheters of the delivery system) as represented in FIG. 86. A technician accordingly is aware of force applied to the substrate 722, and an amount of the force, to determine if corrective action is necessary during a procedure.

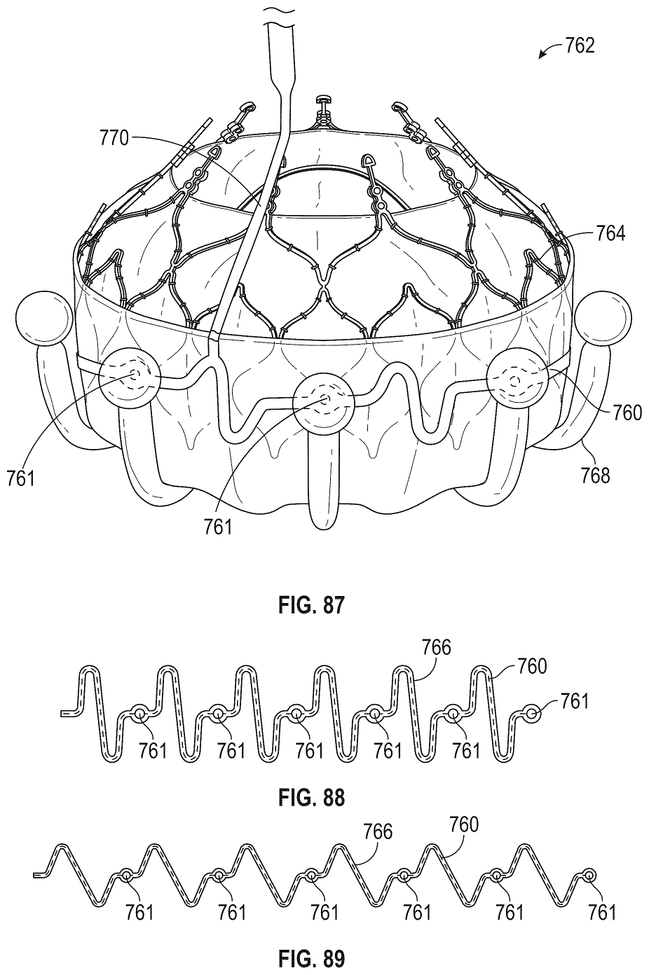

A sensor body as disclosed herein can be implemented with other forms of prosthetic heart implants. FIG. 87 illustrates an implementation of a sensor body 760 configured similarly as any of the sensor bodies shown in FIGS. 82-84, yet coupled to a prosthetic heart valve 762 and including a plurality of sensors 761. The prosthetic heart valve 762 is configured similarly as a prosthetic heart valve 10 illustrated in FIGS. 1A-2. The sensor body 760 extends circumferentially about the prosthetic heart valve 762. Sensors 761 are configured similarly as the sensor 724 and are adapted to detect contact between the sensors 761 and heart tissue. The sensors 761 are linked by a single electrical conduit or trace, or multiple electrical conduits or traces can be utilized.

Referring to FIGS. 88 and 89, the sensor body 760 has an undulating shape that is adapted to expand radially outward with expansion of the prosthetic heart valve 762. The undulating shape is shown in a compressed configuration in FIG. 88 and is shown in an expanded or stretched configuration in FIG. 89. The substrate 766 has the undulating shape that is adapted to expand radially outward with the prosthetic heart valve 762.

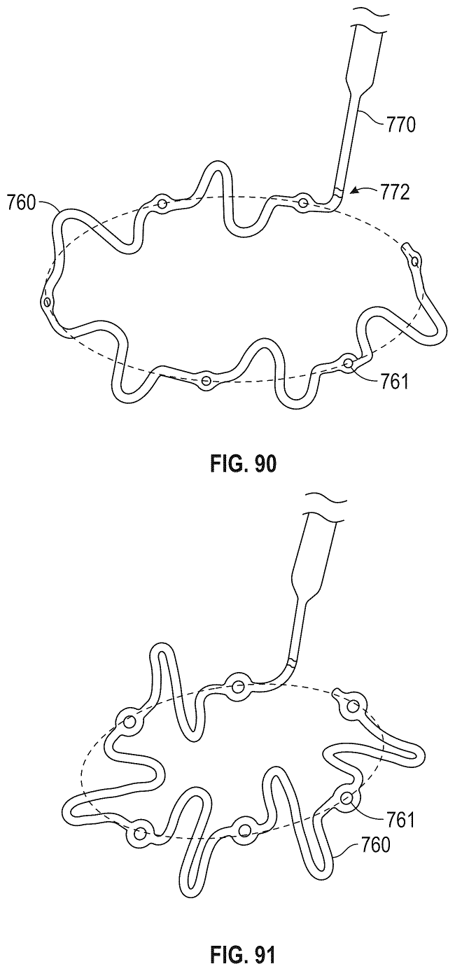

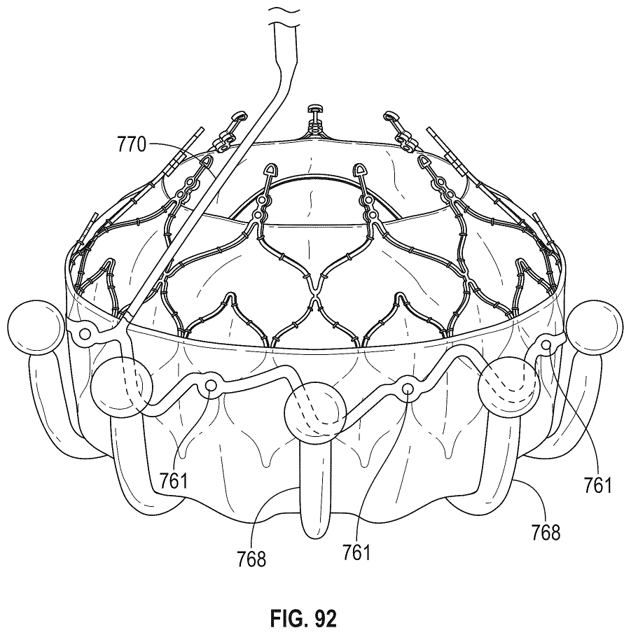

FIG. 90 illustrates the sensor body 760 isolated from the prosthetic heart valve 762 and extending circumferentially in the expanded configuration. FIG. 91 illustrates the sensor body 760 isolated from the prosthetic heart valve 762 and in the compressed configuration.

Referring to FIG. 87, the sensors 761 are positioned to detect heart valve tissue between the anchors and the valve body 764. The sensors 761 accordingly detect whether capture of a leaflet has occurred by an anchor 768, which can be configured similar to other forms of anchors disclosed herein. Sensing tissue between the anchor 768 and the valve body 764 indicates capture. An electrical signal can be passed along an electrical conduit 770 of a delivery system, which can be configured similarly as other forms of electrical conduits disclosed herein. In examples, a disconnect portion 772 (marked in FIG. 90) of the sensor body 760 or the electrical conduit 770 allows for disconnection of the electrical conduit 770. The disconnect portion 772 allows for tear of the connection due to a force applied, yet other manners of disconnection can be utilized in examples (e.g., separation of terminals, magnetic release, among others).

Other positions of sensors 761 can be utilized. FIG. 92 illustrates a variation in which the sensors 761 are circumferentially offset from the position of the anchors 768 (circumferentially between the anchors 768). The position can produce an indication of sealing with the native valve annulus.

The examples of prosthetic valves may be utilized in a mitral valve as disclosed herein, or may be utilized in other deployment locations such as a native tricuspid valve, or other deployment locations. Deployment to aortic or pulmonary valves, or other implantation sites may be utilized.

Various modifications of the examples disclosed herein may be provided. Features of examples may be modified, substituted, excluded, or combined across examples as desired. Combinations of features across examples may be provided as desired. Combinations of features may be provided across examples with other features of such examples being excluded if desired.

The various examples of sealing skirts disclosed herein may have a variety of forms, including cloth skirts, foam skirts, or braided skirts as desired. Various materials may be utilized as desired.

The implants disclosed herein may include prosthetic heart valves or other forms of implants, such as stents or filters, or diagnostic devices, among others. The implants may be expandable implants adapted to move from a compressed or undeployed state to an expanded or deployed state. The implants may be compressible implants adapted to be compressed inward to have a reduced outer profile and to move the implant to the compressed or undeployed state.

Various forms of delivery apparatuses may be utilized with the examples disclosed herein. The delivery apparatuses as disclosed herein may be utilized for aortic, mitral, tricuspid, and pulmonary replacement and repair as well. The delivery apparatuses may comprise delivery apparatuses for delivery of other forms of implants, such as stents or filters, or diagnostic devices, among others.

The implants and the systems disclosed herein may be used in transcatheter mitral or tricuspid implantation, as well as aortic valve implantation (TAVI) or replacement of other native heart valves (e.g., pulmonary valves). The delivery apparatuses and the systems disclosed herein may be utilized for transarterial access, including transfemoral access, to a patient's heart. The delivery apparatuses and systems may be utilized in transcatheter percutaneous procedures, including transarterial procedures, which may be transfemoral. Transapical procedures, among others, may also be utilized. Other procedures may be utilized as desired.

In addition, the methods herein are not limited to the methods specifically described, and may include methods of utilizing the systems and apparatuses disclosed herein. The steps of the methods may be modified, excluded, or added to, with systems, apparatuses, and methods disclosed herein. The examples disclosed herein may comprise systems for implantation within a human body in examples.

For purposes of this description, certain aspects, advantages, and novel features of the examples of this disclosure are described herein. The disclosed methods, apparatuses, and systems should not be construed as limiting in any way. Instead, the present disclosure is directed toward all novel and nonobvious features and aspects of the various disclosed examples, along and in various combinations and sub-combinations with one another. The methods, apparatuses, and systems are not limited to any specific aspect or feature or combination thereof, nor do the disclosed examples require that any one or more specific advantages be present or problems be solved. Features, elements, or combinations of one example can be combined into other examples herein.

Example 1: A prosthetic valve for deployment in a native valve, the prosthetic valve comprising: a valve body; one or more prosthetic valve leaflets coupled to the valve body; one or more anchors adapted to anchor the valve body to the native valve by capturing a native valve leaflet; and an indicator adapted to indicate capture of the native valve leaflet by the one or more anchors.

Example 2: The prosthetic valve of any example herein, in particular example 1, wherein the indicator is visible under imaging.

Example 3: The prosthetic valve of any example herein, in particular example 1 or example 2, wherein an appearance of the indicator varies to indicate capture of the native valve leaflet.

Example 4: The prosthetic valve of any example herein, in particular examples 1-3, wherein a visibility of the indicator is reduced under ultrasound imaging to indicate capture of the native valve leaflet.

Example 5: The prosthetic valve of any example herein, in particular examples 1-4, wherein the indicator moves to indicate capture of the native valve leaflet.

Example 6: The prosthetic valve of any example herein, in particular examples 1-5, wherein the indicator is positioned on the one or more anchors.

Example 7: The prosthetic valve of any example herein, in particular examples 1-6, wherein the indicator includes one or more elongate bodies.

Example 8: The prosthetic valve of any example herein, in particular example 7, wherein the one or more elongate bodies are positioned on the one or more anchors.

Example 9: The prosthetic valve of any example herein, in particular example 7 or example 8, wherein each of the one or more elongate bodies comprises an axially compressible structure, such as a spring.

Example 10: The prosthetic valve of any example herein, in particular examples 7-9, wherein the one or more elongate bodies are positioned on the valve body.

Example 11: The prosthetic valve of any example herein, in particular examples 1-10, wherein the indicator comprises a ring extending circumferentially about the valve body.

Example 12: The prosthetic valve of any example herein, in particular examples 1-11, wherein the indicator extends axially along the valve body.

Example 13: The prosthetic valve of any example herein, in particular examples 1-12, wherein the indicator comprises a loop.

Example 14: The prosthetic valve of any example herein, in particular examples 1-13, wherein the indicator extends from the valve body to at least one of the one or more anchors.

Example 15: The prosthetic valve of any example herein, in particular examples 1-14, wherein the indicator comprises a button adapted to be depressed to indicate capture of the native valve leaflet.

Example 16: The prosthetic valve of any example herein, in particular examples 1-15, wherein the indicator includes a bladder adapted to be filled with contrast material.

Example 17: The prosthetic valve of any example herein, in particular example 16, wherein the bladder includes an opening adapted to release the contrast material to indicate capture of the native valve leaflet.

Example 18: The prosthetic valve of any example herein, in particular examples 1-17, wherein the indicator includes a channel adapted to pass contrast material therethrough.

Example 19: The prosthetic valve of any example herein, in particular examples 1-18, wherein the indicator comprises a sensor.

Example 20: The prosthetic valve of any example herein, in particular examples 1-19, wherein the prosthetic valve is adapted to be deployed to a mitral valve or a tricuspid valve.

Example 21: A method comprising: deploying a prosthetic valve to a native valve, the prosthetic valve including: a valve body, one or more prosthetic valve leaflets coupled to the valve body, one or more anchors adapted to anchor the valve body to the native valve by capturing a native valve leaflet, and an indicator adapted to indicate capture of the native valve leaflet by the one or more anchors.

Example 22: The method of any example herein, in particular example 21, wherein the indicator is adapted to indicate capture of the native valve leaflet under imaging.

Example 23: The method of any example herein, in particular example 21 or example 22, wherein an appearance of the indicator under imaging varies to indicate capture of the native valve leaflet.

Example 24: The method of any example herein, in particular examples 21-23, wherein the indicator is adapted to move to indicate capture of the native valve leaflet.

Example 25: The method of any example herein, in particular examples 21-24, wherein the indicator is positioned on the one or more anchors.

Example 26: The method of any example herein, in particular examples 21-25, wherein the indicator includes one or more elongate bodies.

Example 27: The method of any example herein, in particular example 26, wherein the one or more elongate bodies are positioned on the valve body.

Example 28: The method of any example herein, in particular examples 21-27, wherein the indicator includes a bladder adapted to be filled with contrast material.

Example 29: The method of any example herein, in particular examples 21-28, wherein the indicator comprises a sensor.

Example 30: The method of any example herein, in particular examples 21-29, wherein the native valve is a mitral valve or a tricuspid valve.

Example 31: A sensor system comprising: a prosthetic heart valve for deployment to a native valve of a patient's heart; and one or more sensors adapted to be coupled to the prosthetic heart valve and adapted to detect a condition within the patient's body.

Example 32: The sensor system of any example herein, in particular example 31, wherein the prosthetic heart valve includes a valve body and a plurality of anchors adapted to capture native valve leaflets in a space between the anchors and the valve body for securing the prosthetic heart valve in the heart.

Example 33: The sensor system of any example herein, in particular example 32, wherein at least one of the sensors is positioned on the one or more anchors.

Example 34: The sensor system of any example herein, in particular example 33, wherein at least one of the sensors is positioned on a tip of the one or more anchors.

Example 35: The sensor system of any example herein, in particular examples 32-34, wherein at least one of the sensors is positioned on the valve body.

Example 36: The sensor system of any example herein, in particular examples 31-35, wherein the one or more sensors comprise one or more of a proximity sensor, a contact sensor, a force sensor, an optical sensor, or a chemical sensor.

Example 37: The sensor system of any example herein, in particular examples 31-36, wherein the condition comprises a pressure within at least one chamber of the heart.

Example 38: The sensor system of any example herein, in particular examples 31-37, wherein the condition comprises a pressure differential across the prosthetic heart valve.

Example 39: The sensor system of any example herein, in particular examples 31-38, wherein the condition comprises a temperature within at least one chamber of the heart.

Example 40: The sensor system of any example herein, in particular examples 31-39, wherein the condition comprises a fluid flow within at least one chamber of the heart.

Example 41: The sensor system of any example herein, in particular examples 31-40, wherein the condition comprises a force applied by the prosthetic heart valve to at least a portion of the heart.

Example 42: The sensor system of any example herein, in particular examples 31-41, further comprising a wireless transmitter for transmitting signals from the one or more sensors to a receiver.

Example 43: The sensor system of any example herein, in particular examples 31-42, further comprising a power source for powering the one or more sensors.

Example 44: The sensor system of any example herein, in particular examples 31-43, wherein the one or more sensors are adapted to be clipped to the prosthetic heart valve.

Example 45: The sensor system of any example herein, in particular example 44, wherein the prosthetic heart valve includes a frame, and the one or more sensors are adapted to be clipped to the frame.

Example 46: The sensor system of any example herein, in particular examples 31-45, wherein the one or more sensors include a first sensor and a second sensor, the first sensor adapted to detect a condition within an atrium of the patient's heart, and the second sensor adapted to detect a condition within a ventricle of the patient's heart.

Example 47: The sensor system of any example herein, in particular examples 31-46, further comprising a first electrical terminal for electrically connecting the sensor to a second electrical terminal on a delivery apparatus for the prosthetic heart valve.

Example 48: The sensor system of any example herein, in particular example 47, wherein the prosthetic heart valve includes a valve body supporting one or more prosthetic valve leaflets, and the prosthetic heart valve further comprises an electrical conduit extending along the valve body and connecting the sensor to the first electrical terminal.

Example 49: The sensor system of any example herein, in particular examples 31-48, wherein the prosthetic heart valve includes an inner body and a sealing body positioned radially outward of the inner body and adapted to seal fluid flow with the native valve, and the one or more sensors are positioned between the inner body and the sealing body.

Example 50: The sensor system of any example herein, in particular examples 31-49, wherein the prosthetic heart valve is adapted to be deployed to a mitral valve or a tricuspid valve.

Example 51: The sensor system of any example herein, in particular examples 31-50, further comprising a substrate, and wherein the one or more sensors are positioned on the substrate, and the sensor system further comprises an electrical detection trace positioned on the substrate and adapted to detect a force applied to the substrate.

Example 52: The sensor system of any example herein, in particular example 51, wherein the prosthetic heart valve is adapted to expand radially from a compressed configuration to an expanded configuration, and wherein the substrate is adapted to expand radially outward with the prosthetic heart valve.

Example 53: The sensor system of any example herein, in particular example 52, wherein the substrate has an undulating shape that is adapted to expand radially outward with the prosthetic heart valve.

Example 54: The sensor system of any example herein, in particular examples 51-53, wherein the one or more sensors include a plurality of the sensors positioned on the substrate and electrically linked with an electrical conduit positioned on the substrate.

Example 55: The sensor system of any example herein, in particular examples 51-54, wherein the electrical detection trace includes a disconnect portion adapted to disconnect with an electrical conduit of a delivery apparatus.

Example 56: A method comprising: deploying a sensor system to a native valve, the sensor system including: a prosthetic heart valve for deployment to a native valve of a patient's heart, and one or more sensors adapted to be coupled to the prosthetic heart valve and adapted to detect a condition within the patient's body.

Example 57: The method of any example herein, in particular example 56, wherein the prosthetic heart valve includes a valve body and one or more anchors adapted to anchor the valve body to the native valve by capturing a native valve leaflet, and the condition comprises whether at least one of the anchors has captured the native valve leaflet.

Example 58: The method of any example herein, in particular example 57, wherein at least one of the sensors is positioned on the one or more anchors.

Example 59: The method of any example herein, in particular examples 56-58, wherein the one or more sensors comprise one or more of a proximity sensor, a contact sensor, a force sensor, an optical sensor, or a chemical sensor.

Example 60: The method of any example herein, in particular examples 56-59, wherein the condition comprises a pressure within at least one chamber of the heart.

Example 61: The method of any example herein, in particular examples 56-60, wherein the condition comprises a temperature within at least one chamber of the heart.

Example 62: The method of any example herein, in particular examples 56-61, wherein the condition comprises a fluid flow within at least one chamber of the heart.

Example 63: The method of any example herein, in particular examples 56-62, wherein the condition comprises a force applied by the prosthetic heart valve to at least a portion of the heart.

Example 64: The method of any example herein, in particular examples 56-63, wherein a wireless transmitter is for transmitting signals from the one or more sensors to a receiver.

Example 65: The method of any example herein, in particular examples 56-64, wherein the prosthetic heart valve is for deployment to a mitral valve or a tricuspid valve.

Example 66: A delivery system for delivering an implant to a native heart valve, the delivery system comprising: a delivery apparatus for delivering the implant to the native heart valve; and one or more sensors coupled to the delivery apparatus and adapted to sense a spatial relationship between the delivery apparatus and at least a portion of the native heart valve.

Example 67: The delivery system of any example herein, in particular example 66, wherein the delivery apparatus includes a capsule adapted to retract for release of the implant, and wherein the one or more sensors are coupled to the capsule.

Example 68: The delivery system of any example herein, in particular example 67, wherein the one or more sensors comprise a plurality of the sensors spaced circumferentially from each other about the capsule.

Example 69: The delivery system of any example herein, in particular example 67, wherein the capsule has a length, and the one or more sensors comprise a plurality of the sensors spaced from each other along the length.

Example 70: The delivery system of any example herein, in particular example 69, wherein the plurality of the sensors is aligned with each other along the length of the capsule.

Example 71: The delivery system of any example herein, in particular examples 66-70, wherein the one or more sensors are adapted to sense contact between the delivery apparatus and the portion of the native heart valve.

Example 72: The delivery system of any example herein, in particular examples 66-71, wherein the one or more sensors are adapted to sense contact between the delivery apparatus and one or more native leaflets of the native heart valve.

Example 73: The delivery system of any example herein, in particular examples 66-72, wherein the one or more sensors comprise a plurality of the sensors, and a first sensor of the plurality of sensors is adapted to provide a signal indicating contact with the portion of the native heart valve, and a second sensor of the plurality of sensors is adapted to indicate a lack of contact with a portion of the native heart valve simultaneously with the first sensor providing the signal.

Example 74: The delivery system of any example herein, in particular examples 66-73, further comprising an indicator device adapted to produce an indication of the spatial relationship between the delivery apparatus and at least a portion of the native heart valve sensed by the one or more sensors.

Example 75: The delivery system of any example herein, in particular example 74, wherein the indicator device includes one or more of a visual indicator, a haptic indicator, or an audible indicator.

Example 76: The delivery system of any example herein, in particular example 74 or example 75, wherein the indicator device is adapted to indicate a depth of the delivery apparatus relative to the native heart valve.

Example 77: The delivery system of any example herein, in particular examples 74-76, wherein the indicator device is adapted to indicate a missed capture of a native leaflet by the implant.

Example 78: The delivery system of any example herein, in particular examples 66-77, further comprising a processor for receiving one or more signals from the one or more sensors, the processor for determining a depth of the delivery apparatus relative to the native heart valve based on the one or more signals.

Example 79: The delivery system of any example herein, in particular examples 66-78, further comprising a processor for receiving one or more signals from the one or more sensors, the processor for determining a missed capture of a native leaflet by the implant based on the one or more signals.

Example 80: The delivery system of any example herein, in particular examples 66-79, further comprising the implant, wherein the implant comprises a prosthetic heart valve.

Example 81: A method comprising: delivering an implant to a native heart valve utilizing a delivery system, wherein the delivery system includes: a delivery apparatus for delivering the implant to the native heart valve, and one or more sensors coupled to the delivery apparatus for sensing a spatial relationship between the delivery apparatus and at least a portion of the native heart valve.

Example 82: The method of any example herein, in particular example 81, wherein the delivery apparatus includes a capsule adapted to retract for release of the implant, and wherein the one or more sensors are coupled to the capsule.

Example 83: The method of any example herein, in particular example 82, wherein the one or more sensors comprise a plurality of the sensors spaced circumferentially from each other about the capsule.

Example 84: The method of any example herein, in particular example 82, wherein the capsule has a length, and the one or more sensors comprise a plurality of the sensors spaced from each other along the length.

Example 85: The method of any example herein, in particular examples 81-84, wherein the one or more sensors are adapted to sense contact between the delivery apparatus and the portion of the native heart valve.

Example 86: The method of any example herein, in particular examples 81-85, wherein the one or more sensors are adapted to sense contact between the delivery apparatus and one or more native leaflets of the native heart valve.

Example 87: The method of any example herein, in particular examples 81-86, wherein an indicator device is adapted to produce an indication of the spatial relationship between the delivery apparatus and at least a portion of the native heart valve sensed by the one or more sensors.

Example 88: The method of any example herein, in particular example 87, wherein the indicator device includes one or more of a visual indicator, a haptic indicator, or an audible indicator.

Example 89: The method of any example herein, in particular examples 81-88, wherein a processor is for receiving one or more signals from the one or more sensors, the processor for determining a depth of the delivery apparatus relative to the native heart valve based on the one or more signals.

Example 90: The method of any example herein, in particular examples 81-89, wherein a processor is for receiving one or more signals from the one or more sensors, the processor for determining a missed capture of a native leaflet by the implant based on the one or more signals.

Example 91: A delivery system for delivering an implant to a native heart valve, the delivery system comprising: a delivery apparatus for delivering the implant to the native heart valve; and an imaging apparatus coupled to the delivery apparatus and adapted to image an area external to the delivery apparatus.

Example 92: The delivery system of any example herein, in particular example 91, wherein the imaging apparatus comprises an ultrasound imaging apparatus.

Example 93: The delivery system of any example herein, in particular example 91 or example 92, wherein the imaging apparatus comprises an optical coherence tomography imaging apparatus.

Example 94: The delivery system of any example herein, in particular examples 91-93, wherein the imaging apparatus is adapted to image at least a portion of the native heart valve.

Example 95: The delivery system of any example herein, in particular examples 91-94, wherein the imaging apparatus is adapted to image one or more leaflets of the native heart valve.

Example 96: The delivery system of any example herein, in particular examples 91-95, wherein the delivery apparatus includes an elongate sheath, and the imaging apparatus is adapted to extend within the elongate sheath.

Example 97: The delivery system of any example herein, in particular example 96, wherein the elongate sheath includes a distal end, and the imaging apparatus is adapted to protrude from the distal end of the elongate sheath.

Example 98: The delivery system of any example herein, in particular example 97, wherein the elongate sheath includes a capsule for surrounding the implant, and the capsule includes the distal end of the elongate sheath.

Example 99: The delivery system of any example herein, in particular examples 91-98, wherein the imaging apparatus comprises a catheter.

Example 100: The delivery system of any example herein, in particular examples 91-99, wherein the implant comprises a prosthetic heart valve having a flow channel, and the imaging apparatus is adapted to be positioned within the flow channel of the prosthetic heart valve.

Example 101: The delivery system of any example herein, in particular examples 91-100, further comprising the implant, wherein the implant comprises a prosthetic heart valve having a valve body and one or more anchors for extending over a distal tip of a native valve leaflet.

Example 102: The delivery system of any example herein, in particular example 101, wherein the imaging apparatus is adapted to image through the valve body of the prosthetic heart valve.

Example 103: The delivery system of any example herein, in particular example 101 or example 102, wherein the valve body includes one or more imaging windows for the imaging apparatus to image through.

Example 104: The delivery system of any example herein, in particular example 103, wherein the one or more anchors includes a plurality of the anchors spaced circumferentially from each other, and the one or more imaging windows include a plurality of the imaging windows spaced circumferentially from each other and each positioned at one of the anchors.

Example 105: The delivery system of any example herein, in particular examples 91-104, wherein the delivery apparatus is adapted to deliver the implant to a mitral valve or a tricuspid valve.

Example 106: A method comprising: delivering an implant to a native heart valve utilizing a delivery system, the delivery system including: a delivery apparatus for delivering the implant to the native heart valve, and an imaging apparatus coupled to the delivery apparatus and adapted to image an area external to the delivery apparatus.

Example 107: The method of any example herein, in particular example 106, wherein the imaging apparatus comprises an ultrasound imaging apparatus.

Example 108: The method of any example herein, in particular example 106 or example 107, wherein the imaging apparatus comprises an optical coherence tomography imaging apparatus.

Example 109: The method of any example herein, in particular examples 106-108, wherein the imaging apparatus is adapted to image at least a portion of the native heart valve.

Example 110: The method of any example herein, in particular examples 106-109, wherein the imaging apparatus is adapted to image one or more leaflets of the native heart valve.

Example 111: The method of any example herein, in particular examples 106-110, wherein the delivery apparatus includes an elongate sheath, and the imaging apparatus is adapted to extend within the elongate sheath.

Example 112: The method of any example herein, in particular example 111, wherein the elongate sheath includes a distal end, and the imaging apparatus is adapted to protrude from the distal end of the elongate sheath.

Example 113: The method of any example herein, in particular example 112, wherein the elongate sheath includes a capsule for surrounding the implant, and the capsule includes the distal end of the elongate sheath.

Example 114: The method of any example herein, in particular examples 106-113, wherein the imaging apparatus comprises a catheter.

Example 115: The method of any example herein, in particular examples 106-114, wherein the implant comprises a prosthetic heart valve having a flow channel, and the imaging apparatus is adapted to be positioned within the flow channel of the prosthetic heart valve.

Example 116: A sensor system comprising: a sensor; and one or more anchors coupled to the sensor and adapted to engage an interior heart wall of a chamber of a heart to anchor the sensor to the interior heart wall.

Example 117: The sensor system of any example herein, in particular example 116, wherein the one or more anchors include a clip.

Example 118: The sensor system of any example herein, in particular example 117, wherein the clip includes a first arm and a second arm, the first arm and the second arm adapted to compress tissue of the interior heart wall between the first arm and the second arm.

Example 119: The sensor system of any example herein, in particular example 118, further comprising a support body, wherein the first arm and the second arm are each adapted to pivot relative to the support body.

Example 120: The sensor system of any example herein, in particular example 119, wherein the support body comprises a housing for the sensor.

Example 121: The sensor system of any example herein, in particular examples 118-120, wherein the first arm or the second arm include one or more penetrating bodies for penetrating the interior heart wall.

Example 122: The sensor system of any example herein, in particular examples 116-121, wherein the one or more anchors are adapted to penetrate the interior heart wall.

Example 123: The sensor system of any example herein, in particular examples 116-122, wherein the one or more anchors include a threaded body adapted to penetrate the interior heart wall.

Example 124: The sensor system of any example herein, in particular examples 116-123, wherein the sensor is adapted to sense a condition within the chamber of the heart.

Example 125: The sensor system of any example herein, in particular examples 116-124, wherein the sensor is adapted to sense a property of fluid within the chamber of the heart.

Example 126: The sensor system of any example herein, in particular examples 116-125, wherein the sensor comprises a pressure sensor.

Example 127: The sensor system of any example herein, in particular examples 116-126, wherein the sensor is adapted to protrude into the chamber of the heart with the one or more anchors engaged to the interior heart wall.

Example 128: The sensor system of any example herein, in particular examples 116-127, further comprising a wireless transmitter for transmitting signals from the sensor to a receiver.

Example 129: The sensor system of any example herein, in particular examples 116-128, further comprising a power source for powering the sensor.

Example 130: The sensor system of any example herein, in particular examples 116-129, wherein the sensor is adapted to be positioned in a plurality of locations along the interior heart wall.

Example 131: A method comprising: deploying a sensor system to a native valve, the sensor system including: a sensor, and one or more anchors coupled to the sensor and adapted to engage an interior heart wall of a chamber of a heart to anchor the sensor to the interior heart wall.

Example 132: The method of any example herein, in particular example 131, wherein the one or more anchors include a clip.

Example 133: The method of any example herein, in particular example 132, wherein the clip includes a first arm and a second arm, the first arm and the second arm adapted to compress tissue of the interior heart wall between the first arm and the second arm.

Example 134: The method of any example herein, in particular example 133, wherein the first arm or the second arm include one or more penetrating bodies for penetrating the interior heart wall.

Example 135: The method of any example herein, in particular examples 131-134, wherein the one or more anchors are adapted to penetrate the interior heart wall.

Example 136: The method of any example herein, in particular examples 131-135, wherein the one or more anchors include a threaded body adapted to penetrate the interior heart wall.

Example 137: The method of any example herein, in particular examples 131-136, wherein the sensor is adapted to sense a condition within the chamber of the heart.

Example 138: The method of any example herein, in particular examples 131-137, wherein the sensor is adapted to sense a property of fluid within the chamber of the heart.

Example 139: The method of any example herein, in particular examples 131-138, wherein the sensor comprises a pressure sensor.

Example 140: The method of any example herein, in particular examples 131-139, wherein the sensor is adapted to protrude into the chamber of the heart with the one or more anchors engaged to the interior heart wall.

Example 141: A system comprising: a prosthetic heart valve for deployment to a native valve of a patient's heart, at least a portion of the prosthetic heart valve comprising a pacemaker electrical conduit adapted to conduct an electrical signal for pacing a heart.

Example 142: The system of any example herein, in particular example 141, wherein the prosthetic heart valve includes one or more anchors adapted to anchor the prosthetic heart valve to the native valve, and the pacemaker electrical conduit comprises at least a portion of the one or more anchors.

Example 143: The system of any example herein, in particular example 142, wherein the prosthetic heart valve includes a valve body supporting one or more prosthetic valve leaflets, and the one or more anchors extend radially outward from the valve body.

Example 144: The system of any example herein, in particular example 142 or example 143, wherein the one or more anchors are adapted to extend over a distal tip of a native valve leaflet.

Example 145: The system of any example herein, in particular examples 141-144, further comprising a first electrical terminal for electrically connecting the pacemaker electrical conduit to a second electrical terminal of a pacemaker.

Example 146: The system of any example herein, in particular example 145, wherein the prosthetic heart valve includes a proximal end portion and a distal end portion, and the first electrical terminal is positioned at the proximal end portion of the prosthetic heart valve.

Example 147: The system of any example herein, in particular example 145 or example 146, wherein the prosthetic heart valve includes a frame, and the first electrical terminal is coupled to the frame.

Example 148: The system of any example herein, in particular examples 141-147, wherein the prosthetic heart valve includes a valve body supporting one or more prosthetic valve leaflets, and the pacemaker electrical conduit includes a first portion extending along the valve body and a second portion extending radially outward from the valve body.

Example 149: The system of any example herein, in particular example 148, wherein the second portion includes a tip of the pacemaker electrical conduit.

Example 150: The system of any example herein, in particular example 148 or example 149, wherein the valve body includes a frame, and the first portion of the pacemaker electrical conduit extends along the frame.

Example 151: The system of any example herein, in particular examples 148-150, wherein the second portion is adapted to contact a surface of the heart.

Example 152: The system of any example herein, in particular examples 148-151, wherein the second portion comprises a coil.

Example 153: The system of any example herein, in particular examples 141-152, wherein the prosthetic heart valve includes one or more prosthetic valve leaflets adapted to allow flow in a distal direction, and the pacemaker electrical conduit is adapted to resist a proximal force applied to the prosthetic heart valve.

Example 154: The system of any example herein, in particular examples 141-153, wherein the prosthetic heart valve includes an inner body and a sealing body positioned radially outward of the inner body and adapted to seal fluid flow with the native valve.

Example 155: The system of any example herein, in particular examples 141-154, wherein the prosthetic heart valve is adapted to be deployed to a mitral valve or a tricuspid valve.

Example 156: A method comprising: deploying a prosthetic heart valve to a native valve of a patient's heart, at least a portion of the prosthetic heart valve comprising a pacemaker electrical conduit adapted to conduct an electrical signal for pacing a heart.

Example 157: The method of any example herein, in particular example 156, wherein the prosthetic heart valve includes one or more anchors adapted to anchor the prosthetic heart valve to the native valve, and the pacemaker electrical conduit comprises at least a portion of the one or more anchors.

Example 158: The method of any example herein, in particular example 157, wherein the prosthetic heart valve includes a valve body supporting one or more prosthetic valve leaflets, and the one or more anchors extend radially outward from the valve body.

Example 159: The method of any example herein, in particular example 157 or example 158, wherein the one or more anchors are adapted to extend over a distal tip of a native valve leaflet.

Example 160: The method of any example herein, in particular examples 156-159, wherein a first electrical terminal is for electrically connecting the pacemaker electrical conduit to a second electrical terminal of a pacemaker.

Example 161: The method of any example herein, in particular example 160, wherein the prosthetic heart valve includes a proximal end portion and a distal end portion, and the first electrical terminal is positioned at the proximal end portion of the prosthetic heart valve.

Example 162: The method of any example herein, in particular example 160 or example 161, wherein the prosthetic heart valve includes a frame, and the first electrical terminal is coupled to the frame.

Example 163: The method of any example herein, in particular examples 156-162, wherein the prosthetic heart valve includes a valve body supporting one or more prosthetic valve leaflets, and the pacemaker electrical conduit includes a first portion extending along the valve body and a second portion extending radially outward from the valve body.

Example 164: The method of any example herein, in particular example 163, wherein the second portion includes a tip of the pacemaker electrical conduit.

Example 165: The method of any example herein, in particular example 163 or example 164, wherein the valve body includes a frame, and the first portion of the pacemaker electrical conduit extends along the frame.

Example 166: A system comprising: a prosthetic heart valve for deployment to a native valve of a patient's heart, the prosthetic heart valve including one or more anchors adapted to hook around one or more native valve leaflets to anchor the prosthetic heart valve to the native valve; a delivery catheter for delivering the prosthetic heart valve to the native valve; and a retainer mechanism adapted to retain the one or more native valve leaflets in a contracted state upon the one or more anchors at least partially hooking around the one or more native valve leaflets.

Example 167: The system of any example herein, in particular example 166, wherein the retainer mechanism includes one or more suction ports for applying a suction force to the one or more native valve leaflets to retain the one or more native valve leaflets in the contracted state.

Example 168: The system of any example herein, in particular example 167, wherein the one or more suction ports are positioned on the delivery catheter.

Example 169: The system of any example herein, in particular examples 166-168, wherein the retainer mechanism includes a coil for extending around a radially outward facing surface of the one or more native valve leaflets.

Example 170: The system of any example herein, in particular example 169, wherein the coil is adapted to deploy from the delivery catheter.

Example 171: The system of any example herein, in particular examples 166-170, wherein the retainer mechanism includes one or more barbs for engaging the one or more native valve leaflets.

Example 172: The system of any example herein, in particular example 171, wherein the one or more barbs are coupled to one or more arms.

Example 173: The system of any example herein, in particular example 172, wherein the one or more arms are adapted to protrude radially outward from the delivery catheter.

Example 174: The system of any example herein, in particular examples 171-173, wherein the one or more barbs comprise a plurality of the barbs circumferentially spaced from each other.

Example 175: The system of any example herein, in particular examples 171-174, wherein the one or more barbs are coupled to a sheath.

Example 176: The system of any example herein, in particular examples 166-175, wherein the retainer mechanism includes one or more arms adapted to hook around the one or more native valve leaflets.

Example 177: The system of any example herein, in particular example 176, wherein the one or more arms are adapted to retract radially inward.

Example 178: The system of any example herein, in particular example 176 or example 177, wherein the delivery catheter includes a guide wire lumen, and the one or more arms are adapted to protrude radially outward from the guide wire lumen.

Example 179: The system of any example herein, in particular examples 176-178, wherein the delivery catheter includes a nose body, and the one or more arms are adapted to protrude radially outward from the nose body.

Example 180: The system of any example herein, in particular examples 166-179, wherein the prosthetic heart valve comprises a prosthetic mitral heart valve or a prosthetic tricuspid heart valve.

Example 181: A method comprising: deploying a prosthetic heart valve to a native valve of a patient's heart utilizing a delivery catheter, the prosthetic heart valve including one or more anchors adapted to hook around one or more native valve leaflets to anchor the prosthetic heart valve to the native valve; and utilizing a retainer mechanism to retain the one or more native valve leaflets in a contracted state when the one or more anchors at least partially hook around the one or more native valve leaflets.

Example 182: The method of any example herein, in particular example 181, wherein the retainer mechanism includes one or more suction ports for applying a suction force to the one or more native valve leaflets to retain the one or more native valve leaflets in the contracted state.

Example 183: The method of any example herein, in particular example 181 or example 182, wherein the retainer mechanism includes a coil for extending around a radially outward facing surface of the one or more native valve leaflets.

Example 184: The method of any example herein, in particular examples 181-183, wherein the retainer mechanism includes one or more barbs for engaging the one or more native valve leaflets.

Example 185: The method of any example herein, in particular examples 181-184, wherein the retainer mechanism includes one or more arms adapted to hook around the one or more native valve leaflets.

Example 186: A prosthetic valve for deployment to a native valve, the prosthetic valve comprising: one or more prosthetic valve leaflets; an inner frame supporting the one or more prosthetic valve leaflets and having an inflow end portion and an outflow end portion; a sealing body positioned radially outward of the inner frame and including a plurality of elongate prongs and a skirt, the plurality of elongate prongs each having a first end portion coupled to the inflow end portion of the inner frame and protruding radially outward from the inner frame to a second end portion, the skirt being suspended between the second end portions of the plurality of prongs and an outflow end portion of the prosthetic valve, the skirt bounding a pocket positioned between the skirt and the inner frame; and one or more anchors adapted to anchor the prosthetic valve to the native valve by capturing a native valve leaflet.

Example 187: The prosthetic valve of any example herein, in particular example 186, wherein the one or more anchors are coupled to the outflow end portion of the inner frame and protrude radially outward from the outflow end portion of the inner frame.

Example 188: The prosthetic valve of any example herein, in particular example 186 or example 187, wherein the one or more anchors are adapted to hook around one or more native valve leaflets to anchor the prosthetic valve to the native valve.

Example 189: The prosthetic valve of any example herein, in particular examples 186-188, wherein the elongate prongs are circumferentially spaced about the inflow end portion of the inner frame.

Example 190: The prosthetic valve of any example herein, in particular examples 186-189, wherein the plurality of elongate prongs forms a plateau portion of the sealing body.

Example 191: The prosthetic valve of any example herein, in particular example 190, wherein the second end portions of the plurality of elongate prongs form the outermost portion of the plateau portion.

Example 192: The prosthetic valve of any example herein, in particular examples 186-191, wherein each of the plurality of elongate prongs are deflectable in an axial dimension of the prosthetic valve.

Example 193: The prosthetic valve of any example herein, in particular examples 186-192, wherein the skirt extends along the plurality of elongate prongs from the second end portions of the plurality of elongate prongs to the first end portions of the plurality of elongate prongs.

Example 194: The prosthetic valve of any example herein, in particular examples 186-193, wherein a portion of the skirt extends along the inner frame and includes a plurality of apertures to allow blood to enter the pocket.

Example 195: The prosthetic valve of any example herein, in particular examples 186-194, wherein the prosthetic valve comprises a prosthetic mitral heart valve or a prosthetic tricuspid heart valve.

Example 196: A method comprising: deploying a prosthetic heart valve to a native heart valve, the prosthetic heart valve including: one or more prosthetic valve leaflets, an inner frame supporting the one or more prosthetic valve leaflets and having an inflow end portion and an outflow end portion, a sealing body positioned radially outward of the inner frame and including a plurality of elongate prongs and a skirt, the plurality of elongate prongs each having a first end portion coupled to the inflow end portion of the inner frame and protruding radially outward from the inner frame to a second end portion, the skirt being suspended between the second end portions of the plurality of prongs and an outflow end portion of the prosthetic valve, the skirt bounding a pocket positioned between the skirt and the inner frame, and one or more anchors adapted to anchor the prosthetic heart valve to the native heart valve by capturing a native valve leaflet.

Example 197: The method of any example herein, in particular example 196, wherein the one or more anchors are coupled to the outflow end portion of the inner frame and protrude radially outward from the outflow end portion of the inner frame.

Example 198: The method of any example herein, in particular example 196 or example 197, wherein the one or more anchors are adapted to hook around one or more native valve leaflets to anchor the prosthetic heart valve to the native heart valve.

Example 199: The method of any example herein, in particular examples 196-198, wherein the plurality of elongate prongs is circumferentially spaced from each other about the inflow end portion of the inner frame.

Example 200: The method of any example herein, in particular examples 196-199, wherein the plurality of elongate prongs forms a plateau portion of the sealing body.

Example 201: A prosthetic valve for deployment to a native valve, the prosthetic valve comprising: one or more prosthetic valve leaflets; and a support structure for supporting the one or more prosthetic valve leaflets and including at least one ring coupled to a skirt, the skirt or the at least one ring being adapted to seal with at least a portion of the native valve.

Example 202: The prosthetic valve of any example herein, in particular example 201, wherein the at least one ring is compliant.

Example 203: The prosthetic valve of any example herein, in particular example 201 or example 202, wherein the at least one ring includes a first ring adapted to be positioned on an inflow side of the native valve and a second ring adapted to be positioned on an outflow side of the native valve.

Example 204: The prosthetic valve of any example herein, in particular example 203, wherein the skirt extends between the first ring and the second ring and forms a sheath.

Example 205: The prosthetic valve of any example herein, in particular example 203 or example 204, further comprising a support body coupled to the one or more prosthetic valve leaflets and coupled to the first ring and to the second ring with the skirt.

Example 206: The prosthetic valve of any example herein, in particular example 205, wherein the support body comprises a third ring.

Example 207: The prosthetic valve of any example herein, in particular examples 203-206, further comprising one or more tethers for axially compressing the first ring and the second ring together.

Example 208: The prosthetic valve of any example herein, in particular examples 201-207, wherein the support structure includes an inner frame, and the skirt forms a disk extending radially outward from the inner frame and supported at an outer periphery of the disk by the at least one ring.

Example 209: The prosthetic valve of any example herein, in particular example 208, wherein the disk is adapted to be positioned on an inflow side of the native valve.

Example 210: The prosthetic valve of any example herein, in particular examples 201-209, wherein the support structure includes an inner frame and an outer frame positioned radially outward of the inner frame, and the skirt forms a disk extending radially outward from the outer frame and supported at an outer periphery of the disk by the at least one ring.

Example 211: The prosthetic valve of any example herein, in particular examples 208-210, further comprising one or more anchors adapted to anchor the prosthetic valve to the native valve by capturing a native valve leaflet.

Example 212: The prosthetic valve of any example herein, in particular examples 201-211, wherein the at least one ring is biased radially outward.

Example 213: The prosthetic valve of any example herein, in particular examples 201-212, wherein the at least one ring is adapted to vary in shape.

Example 214: The prosthetic valve of any example herein, in particular examples 201-213, wherein the at least one ring includes a first end and a second end and the first end is adapted to slide relative to the second end to vary a diameter of the at least one ring.

Example 215: The prosthetic valve of any example herein, in particular examples 201-214, wherein the prosthetic valve comprises a prosthetic mitral heart valve or a prosthetic tricuspid heart valve.

Example 216: A method comprising: deploying a prosthetic heart valve to a native heart valve, the prosthetic heart valve including: one or more prosthetic valve leaflets, and a support structure for supporting the one or more prosthetic valve leaflets and including at least one ring coupled to a skirt, the skirt or the at least one ring being adapted to seal with at least a portion of the native valve.

Example 217: The method of any example herein, in particular example 216, wherein the at least one ring is compliant.

Example 218: The method of any example herein, in particular example 216 or example 217, wherein the at least one ring includes a first ring adapted to be positioned on an inflow side of the native heart valve and a second ring adapted to be positioned on an outflow side of the native heart valve.

Example 219: The method of any example herein, in particular example 218, wherein the skirt extends between the first ring and the second ring and forms a sheath.

Example 220: The method of any example herein, in particular examples 216-219, wherein the support structure includes an inner frame, and the skirt forms a disk extending radially outward from the inner frame and supported at an outer periphery of the disk by the at least one ring.

Example 221: A sensor system comprising: a prosthetic heart implant; and a sensor body including: a substrate, a sensor positioned on the substrate and adapted to detect a condition of the prosthetic heart implant, and an electrical detection trace positioned on the substrate and adapted to detect a force applied to the substrate.

Example 222: The sensor system of any example herein, in particular example 221, wherein the electrical detection trace is adapted to detect a partial or full tear of the substrate.

Example 223: The sensor system of any example herein, in particular example 221 or example 222, wherein the electrical detection trace is adapted to provide an electrical signal indicating an amount of the force applied to the substrate.

Example 224: The sensor system of any example herein, in particular examples 221-223, wherein the substrate comprises a flexible circuit board.

Example 225: The sensor system of any example herein, in particular examples 221-224, wherein the sensor comprises an electrode.

Example 226: The sensor system of any example herein, in particular examples 221-225, wherein the prosthetic heart implant comprises a clip adapted to clip together heart leaflets.

Example 227: The sensor system of any example herein, in particular example 226, wherein the sensor is adapted to detect contact between the clip and the heart leaflets.

Example 228: The sensor system of any example herein, in particular example 226 or example 227, wherein the sensor body comprises a strip that is adapted to be extracted from a portion of the clip.

Example 229: The sensor system of any example herein, in particular example 228, wherein the electrical detection trace is adapted to detect a partial or full tear of the substrate upon in vivo extraction of the sensor body from the portion of the clip.

Example 230: The sensor system of any example herein, in particular examples 227-229, wherein the clip includes one or more arms and the sensor body is positioned on at least one of the one or more arms.

Example 231: The sensor system of any example herein, in particular examples 221-230, wherein the prosthetic heart implant comprises a prosthetic heart valve having one or more anchors adapted to anchor the prosthetic heart implant to a native heart valve, and the sensor is adapted to detect whether at least one of the anchors has captured the native valve leaflet.

Example 232: The sensor system of any example herein, in particular example 231, wherein the prosthetic heart valve is adapted to expand radially from a compressed configuration to an expanded configuration, and wherein the substrate is adapted to expand radially outward with the prosthetic heart valve.

Example 233: The sensor system of any example herein, in particular examples 221-232, wherein the electrical detection trace has a loop shape with a first end and a second end and includes a first electrical terminal at the first end and a second electrical terminal at the second end.

Example 234: The sensor system of any example herein, in particular examples 221-233, wherein the electrical detection trace electrically conducts through the sensor.

Example 235: The sensor system of any example herein, in particular examples 221-234, wherein the electrical detection trace includes a strain gauge on the substrate.

Example 236: A method comprising: deploying a prosthetic heart implant to a native heart valve; and utilizing a sensor body coupled to the prosthetic heart implant to detect a condition of the prosthetic heart implant, the sensor body including: a substrate, a sensor positioned on the substrate and adapted to detect the condition of the prosthetic heart implant, and an electrical detection trace positioned on the substrate and adapted to detect a force applied to the substrate.

Example 237: The method of any example herein, in particular example 236, wherein the electrical detection trace is adapted to detect a partial or full tear of the substrate.

Example 238: The method of any example herein, in particular example 236 or example 237, wherein the electrical detection trace is adapted to provide an electrical signal indicating an amount of the force applied to the substrate.

Example 239: The method of any example herein, in particular examples 236-238, wherein the substrate comprises a flexible circuit board.

Example 240: The method of any example herein, in particular examples 236-239, wherein the sensor comprises an electrode.

Any of the features of any of the examples, including but not limited to any of the first through 240 examples referred to above, is applicable to all other aspects and examples identified herein, including but not limited to any examples of any of the first through 240 examples referred to above. Moreover, any of the features of an example of the various examples, including but not limited to any examples of any of the first through 240 examples referred to above, is independently combinable, partly or wholly with other examples described herein in any way, e.g., one, two, or three or more examples may be combinable in whole or in part. Further, any of the features of the various examples, including but not limited to any examples of any of the first through 240 examples referred to above, may be made optional to other examples. Any example of a method can be performed by a system or apparatus of another example, and any aspect or example of a system or apparatus can be configured to perform a method of another aspect or example, including but not limited to any examples of any of the first through 240 examples referred to above.

In closing, it is to be understood that although aspects of the present specification are highlighted by referring to specific examples, one skilled in the art will readily appreciate that these disclosed examples are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular examples only, and is not intended to limit the scope of systems, apparatuses, and methods as disclosed herein, which is defined solely by the claims. Accordingly, the systems, apparatuses, and methods are not limited to that precisely as shown and described.

Certain examples of systems, apparatuses, and methods are described herein, including the best mode known to the inventors for carrying out the same. Of course, variations on these described examples will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described examples in all possible variations thereof is encompassed by the systems, apparatuses, and methods unless otherwise indicated herein or otherwise clearly contradicted by context.

Groupings of alternative examples, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A sensor system comprising:
a prosthetic heart valve for deployment to a native valve of a patient's heart; and
one or more sensors adapted to be coupled to the prosthetic heart valve and adapted to detect a condition within the patient's body.

2. The sensor system of claim 1, wherein the prosthetic heart valve includes a valve body and a plurality of anchors adapted to capture native valve leaflets in a space between the anchors and the valve body for securing the prosthetic heart valve in the heart.

3. The sensor system of claim 2, wherein at least one of the sensors is positioned on at least one of the plurality of anchors.

4. The sensor system of claim 3, wherein at least one of the sensors is positioned on a tip of at least one of the plurality of anchors.

5. The sensor system of claim 2, wherein at least one of the sensors is positioned on the valve body.

6. The sensor system of claim 2, wherein at least one of the sensors is configured to indicate capture of at least one of the native valve leaflets by at least one of the plurality of anchors.

7. The sensor system of claim 2, further comprising a ring extending circumferentially about the valve body, the ring being configured to move to indicate capture of at least one of the native valve leaflets by at least one of the plurality of anchors.

8. The sensor system of claim 1, wherein the one or more sensors comprise one or more of a proximity sensor, a contact sensor, a force sensor, an optical sensor, or a chemical sensor.

9. The sensor system of claim 1, wherein the condition comprises a pressure within at least one chamber of the heart.

10. The sensor system of claim 1, wherein the condition comprises a fluid flow within at least one chamber of the heart.

11. The sensor system of claim 1, wherein the condition comprises a force applied by the prosthetic heart valve to at least a portion of the heart.

12. The sensor system of claim 1, further comprising a wireless transmitter for transmitting signals from the one or more sensors to a receiver.

13. The sensor system of claim 1, further comprising a power source for powering the one or more sensors.

14. The sensor system of claim 1, further comprising a first electrical terminal for electrically connecting at least one of the one or more sensors to a second electrical terminal on a delivery apparatus for the prosthetic heart valve.

15. A prosthetic valve for deployment to a native valve, the prosthetic valve comprising:
one or more prosthetic valve leaflets;
an inner frame supporting the one or more prosthetic valve leaflets and having an inflow end portion and an outflow end portion;
a sealing body positioned radially outward of the inner frame and including a plurality of elongate prongs and a skirt, the plurality of elongate prongs each having a first end portion coupled to the inflow end portion of the inner frame and protruding radially outward from the inner frame to a second end portion, the skirt being suspended between the second end portions of the plurality of elongate prongs and an outflow end portion of the prosthetic valve, the skirt bounding a pocket positioned between the skirt and the inner frame; and
one or more anchors adapted to anchor the prosthetic valve to the native valve by capturing a native valve leaflet.

16. The prosthetic valve of claim 15, wherein the one or more anchors are adapted to hook around one or more native valve leaflets to anchor the prosthetic valve to the native valve.

17. The prosthetic valve of claim 15, wherein the plurality of elongate prongs forms a plateau portion of the sealing body.

18. A sensor system comprising:
a prosthetic heart implant; and
a sensor body including:
a substrate,
a sensor positioned on the substrate and adapted to detect a condition of the prosthetic heart implant, and
an electrical detection trace positioned on the substrate and adapted to detect a force applied to the substrate.

19. The sensor system of claim 18, wherein the prosthetic heart implant comprises a clip adapted to clip together heart leaflets.

20. The sensor system of claim 19, wherein the electrical detection trace is adapted to detect a partial or full tear of the substrate upon in vivo extraction of the sensor body from a portion of the clip.
